SEQUENCE LISTING (114 sequences)

SEQ ID NO: 1 - protein, 8 aa, Artificial Sequence
  Bacillus cereus B4264 nt 2,341,208 - 2,406,996
  misc_feature (1)..(1): Xaa can be any naturally occurring amino acid
  MISC_FEATURE (2)..(2): 2,3-diaminopropionic acid
XXSASLEE

SEQ ID NO: 2 - protein, 8 aa, Artificial Sequence
  Burkholderia gladioli BSR3, chr 2 nt 1,312,845 - 1,376,223
  misc_feature (1)..(1): Xaa can be any naturally occurring amino acid
  MISC_FEATURE (2)..(2): 2,3-diaminopropionic acid
XXPQAVFP

SEQ ID NO: 3 - protein, 6 aa, Artificial Sequence
  Burkholderia gladioli BSR3, chr 2 nt 2,023,222 - 2,080,171
  misc_feature (1)..(1): Xaa can be any naturally occurring amino acid
  MISC_FEATURE (2)..(2): 2,3-diaminopropionic acid
XXTYVS

SEQ ID NO: 4 - protein, 9 aa, Artificial Sequence
  Burkholderia gladioli BSR3, chr 2 nt 2,103,056 - 2,186,245
  misc_feature (1)..(1): Xaa can be any naturally occurring amino acid
  MISC_FEATURE (3)..(3): 2,3-diaminopropionic acid
XAXSKRKGV

SEQ ID NO: 5 - protein, 13 aa, Artificial Sequence
  Burkholderia pseudomallei K96243, chr 2 nt 2,187,213 - 2,268,799
  misc_feature (1)..(1): Xaa can be any naturally occurring amino acid
  MISC_FEATURE (2)..(2): 2,3-diaminopropionic acid
  MISC_FEATURE (3)..(3): ornithine
  MISC_FEATURE (8)..(8): 2,4-diaminobutyric acid
  MISC_FEATURE (11)..(11): ornithine
XXXSETLXTTXGV

SEQ ID NO: 6 - protein, 13 aa, Artificial Sequence
  Burkholderia pseudomallei K96243, chr 2 nt 2,187,213 - 2,268,799
  misc_feature (1)..(1): Xaa can be any naturally occurring amino acid
  MISC_FEATURE (2)..(2): 2,3-diaminopropionic acid
  MISC_FEATURE (8)..(8): 2,4-diaminobutyric acid
XXESRTLXTTEGV

SEQ ID NO: 7 - protein, 8 aa, Artificial Sequence
  Burkholderia rhizoxinica HKI 454, pBRH01 nt 3,955 - 66,916
  misc_feature (1)..(1): Xaa can be any naturally occurring amino acid
  MISC_FEATURE (3)..(3): 2,3-diaminopropionic acid
XLXYAAAV

SEQ ID NO: 8 - protein, 10 aa, Artificial Sequence
  Clostridium botulinum A2 str. Kyoto nt 338,515 - 416,384
  misc_feature (1)..(1): Xaa can be any naturally occurring amino acid
  MISC_FEATURE (3)..(3): 2,3-diaminopropionic acid
XNXISEVLFL

SEQ ID NO: 9 - protein, 10 aa, Artificial Sequence
  Clostridium cellulovorans p743B nt 2,914,102 - 2,963,633
  misc_feature (1)..(1): Xaa can be any naturally occurring amino acid
  MISC_FEATURE (2)..(2): 2,4-diaminobutyric acid
XXTYSKEYFF

SEQ ID NO: 10 - protein, 10 aa, Artificial Sequence
  Clostridium cellulovorans p743B nt 2,967,242 - 3,025,608
  misc_feature (1)..(1): Xaa can be any naturally occurring amino acid
  MISC_FEATURE (2)..(2): 2,4-diaminobutyric acid
  MISC_FEATURE (4)..(4): 2,4-diaminobutyric acid
XXSXASVDIF

SEQ ID NO: 11 - protein, 7 aa, Artificial Sequence
  Rhodococcus pyridinivorans SB3094 nt 2,309,268 - 2,368,050
  misc_feature (1)..(1): Xaa can be any naturally occurring amino acid
  MISC_FEATURE (4)..(4): 2,3-diaminopropionic acid
XLIXFAY

SEQ ID NO: 12 - protein, 10 aa, Artificial Sequence
  Dickeya dadantii Ech586 nt 3,414,889 - 3,485,467
  misc_feature (1)..(1): Xaa can be any naturally occurring amino acid
  MISC_FEATURE (2)..(2): 2,3-diaminopropionic acid
  MISC_FEATURE (5)..(5): ornithine
  MISC_FEATURE (8)..(8): ornithine
XXNLXSPXFT

SEQ ID NO: 13 - protein, 7 aa, Artificial Sequence
  Herbaspirillum seropedicae SmR1 nt 2,631,343 - 2,698,825
  misc_feature (1)..(1): Xaa can be any naturally occurring amino acid
  MISC_FEATURE (3)..(3): 2,3-diaminopropionic acid
XDXDTSN

SEQ ID NO: 14 - protein, 9 aa, Artificial Sequence
  Mycobacterium marinum M nt 992,526 - 1,058,784
  misc_feature (1)..(1): Xaa can be any naturally occurring amino acid
  MISC_FEATURE (3)..(3): 2,3-diaminopropionic acid
XGXATFGVF

SEQ ID NO: 15 - protein, 10 aa, Artificial Sequence
  Mycobacterium marinum M nt 3,964,578 - 4,014,848
  misc_feature (1)..(1): Xaa can be any naturally occurring amino acid
  MISC_FEATURE (3)..(3): 2,3-diaminopropionic acid
XAXTAGTFTT

SEQ ID NO: 16 - protein, 13 aa, Artificial Sequence
  Mycobacterium rhodesiae NBB3 nt 4,719,612 - 4,799,700
  misc_feature (1)..(1): Xaa can be any naturally occurring amino acid
  MISC_FEATURE (3)..(3): 2,3-diaminopropionic acid
XAXTTTTTRDVTR

SEQ ID NO: 17 - protein, 14 aa, Artificial Sequence
  Nocardia cyriacigeorgica GUH-2 nt 5,347,951 - 5,451,235
  misc_feature (1)..(1): Xaa can be any naturally occurring amino acid
  MISC_FEATURE (2)..(2): 2,3-diaminopropionic acid
XXSSTSSSSSSSWS

SEQ ID NO: 18 - protein, 13 aa, Artificial Sequence
  Paenibacillus mucilaginosus KNP414 nt 3,128,888 - 3,215,024
  misc_feature (1)..(1): Xaa can be any naturally occurring amino acid
  MISC_FEATURE (2)..(2): 2,3-diaminopropionic acid
  MISC_FEATURE (4)..(4): ornithine
  MISC_FEATURE (12)..(12): ornithine
XXTXDGNDGYVXF

SEQ ID NO: 19 - protein, 8 aa, Artificial Sequence
  Paenibacillus mucilaginosus KNP414 nt 5,527,457 - 5,599,870
  misc_feature (1)..(1): Xaa can be any naturally occurring amino acid
  MISC_FEATURE (2)..(2): 2,3-diaminopropionic acid
  MISC_FEATURE (6)..(6): 2,4-diaminobutyric acid
  MISC_FEATURE (8)..(8): ornithine
XXCIFXCX

SEQ ID NO: 20 - protein, 7 aa, Artificial Sequence
  Paenibacillus polymyxa E681 nt 61,938 - 131,860
  misc_feature (1)..(1): Xaa can be any naturally occurring amino acid
  MISC_FEATURE (2)..(2): 2,3-diaminopropionic acid
XXVFTNA

SEQ ID NO: 21 - protein, 6 aa, Artificial Sequence
  Paenibacillus terrae HPL-003 nt 1,241,438 - 1,303,692
IFKSTF

SEQ ID NO: 22 - protein, 10 aa, Artificial Sequence
  Paenibacillus terrae HPL-003 nt 4,207,798 - 4,291,196
  misc_feature (1)..(1): Xaa can be any naturally occurring amino acid
  MISC_FEATURE (3)..(3): ornithine
XVXGSFSPQP

SEQ ID NO: 23 - protein, 11 aa, Artificial Sequence
  Pseudomonas entomophila L48 nt 3,437,580 - 3,517,142
  misc_feature (1)..(1): Xaa can be any naturally occurring amino acid
  MISC_FEATURE (8)..(8): 2,3-diaminopropionic acid
XAVLDGGXTSK

SEQ ID NO: 24 - protein, 7 aa, Artificial Sequence
  Pseudomonas syringae pv. syringae B728a nt 4,391,388 - 4,451,295
  misc_feature (1)..(1): Xaa can be any naturally occurring amino acid
  MISC_FEATURE (3)..(3): 2,3-diaminopropionic acid
XLXDVAS

SEQ ID NO: 25 - protein, 8 aa, Artificial Sequence
  Rhodococcus opacus B4 nt 3,545,422 - 3,611,002
  misc_feature (1)..(1): Xaa can be any naturally occurring amino acid
XLZFLSYY

SEQ ID NO: 26 - protein, 6 aa, Artificial Sequence
  Rhodococcus opacus B4 nt 5,997,386 - 6,053,147
  misc_feature (1)..(1): Xaa can be any naturally occurring amino acid
  MISC_FEATURE (3)..(3): 2,3-diaminopropionic acid
XWXGYR

SEQ ID NO: 27 - protein, 7 aa, Artificial Sequence
  Rhodococcus pyridinivorans SB3094 nt 859,328 - 921,377
  misc_feature (1)..(1): Xaa can be any naturally occurring amino acid
  MISC_FEATURE (3)..(3): 2,3-diaminopropionic acid
XFXATFF

SEQ ID NO: 28 - protein, 6 aa, Artificial Sequence
  Gordonia sp. KTR9 nt 1,029,407 - 1,089,317
  misc_feature (1)..(1): Xaa can be any naturally occurring amino acid
  MISC_FEATURE (3)..(3): 2,3-diaminopropionic acid
XYXAKR

SEQ ID NO: 29 - protein, 8 aa, Artificial Sequence
  Xanthomonas albilineans GPE PC73 nt 836,222 - 899,513
  misc_feature (1)..(1): Xaa can be any naturally occurring amino acid
  MISC_FEATURE (4)..(4): 2,4-diaminobutyric acid
XKFXNAVQ

SEQ ID NO: 30 - protein, 13 aa, Artificial Sequence
  Xanthomonas albilineans GPE PC73 nt 1,166,681 - 1,259,075
  misc_feature (1)..(1): Xaa can be any naturally occurring amino acid
  MISC_FEATURE (3)..(3): ornithine
XGXTNYNYSNDNV

SEQ ID NO: 31 - protein, 7 aa, Artificial Sequence
  Uncultured bacterium contig 00137 nt 1 - 35,392
  misc_feature (1)..(1): Xaa can be any naturally occurring amino acid
  MISC_FEATURE (3)..(3): 2,3-diaminopropionic acid
  misc_feature (5)..(5): Xaa can be any naturally occurring amino acid
  MISC_FEATURE (6)..(6): N-alpha-methylation
  MISC_FEATURE (7)..(7): ornithine
XAXSXXP

SEQ ID NO: 32 - protein, 11 aa, Artificial Sequence
  Uncultured bacterium nt 33,345 - 119,152
  misc_feature (1)..(1): Xaa can be any naturally occurring amino acid
  MISC_FEATURE (3)..(3): 2,3-diaminopropionic acid
XDXVEDDGDVP

SEQ ID NO: 33 - protein, 11 aa, Artificial Sequence
  Uncultured bacterium nt 33,345 - 119,152
  misc_feature (1)..(1): Xaa can be any naturally occurring amino acid
  MISC_FEATURE (5)..(5): ornithine
XDYVXDDGDVP

SEQ ID NO: 34 - protein, 6 aa, Artificial Sequence
  Achromobacter xylosoxidans A8 nt 2,723,596 - 2,795,344
  misc_feature (1)..(1): Xaa can be any naturally occurring amino acid
  MISC_FEATURE (2)..(2): 2,3-diaminopropionic acid
  MISC_FEATURE (5)..(5): ornithine
XXDSXS

SEQ ID NO: 35 - protein, 7 aa, Artificial Sequence
  Amycolicicoccus subflacus DQS3-9A1 nt 4,121,859 - 4,184,019
  misc_feature (1)..(1): Xaa can be any naturally occurring amino acid
  MISC_FEATURE (3)..(3): 2,3-diaminopropionic acid
XYXYTFF

SEQ ID NO: 36 - protein, 11 aa, Artificial Sequence
  Burkholderia rhizoxinica HKI 454 pBRH01 nt 621,904 - 691,902
  misc_feature (1)..(1): Xaa can be any naturally occurring amino acid
  MISC_FEATURE (4)..(4): 2,3-diaminopropionic acid
  MISC_FEATURE (7)..(7): N-alpha-methylation
  MISC_FEATURE (10)..(10): N-alpha-methylation
XVVXSGXVVXS

SEQ ID NO: 37 - protein, 6 aa, Artificial Sequence
  Burkholderia pseudomallei 668 chr.II nt 2,200,921 - 2,258,941
  misc_feature (1)..(1): Xaa can be any naturally occurring amino acid
  MISC_FEATURE (2)..(2): 2,3-diaminopropionic acid
XXEEGV

SEQ ID NO: 38 - protein, 7 aa, Artificial Sequence
  Streptosporangium roseum DMS 43021 nt 7,175,764 - 7,243,639
  misc_feature (1)..(1): Xaa can be any naturally occurring amino acid
  MISC_FEATURE (3)..(3): 2,3-diaminopropionic acid
XIXTFTV

SEQ ID NO: 39 - protein, 7 aa, Artificial Sequence
  Burkholderia sp. Y123 chr.3 nt 965,645 - 1,034,316
  misc_feature (1)..(1): Xaa can be any naturally occurring amino acid
  MISC_FEATURE (2)..(2): ornithine
XXTVEDG

SEQ ID NO: 40 - protein, 6 aa, Artificial Sequence
  Collimonas fungivorans Ter331 nt 291,223 - 348,100
  misc_feature (1)..(1): Xaa can be any naturally occurring amino acid
  MISC_FEATURE (3)..(3): 2,3-diaminopropionic acid
XLXVSI

SEQ ID NO: 41 - protein, 9 aa, Artificial Sequence
  Clostridium botulinum H04402 065 nt 333,496 - 408,715
  misc_feature (1)..(1): Xaa can be any naturally occurring amino acid
  MISC_FEATURE (3)..(3): 2,3-diaminopropionic acid
XNXESACLV

SEQ ID NO: 42 - protein, 9 aa, Artificial Sequence
  Rhodococcus erythropolis CCM2595 nt 3,424,162 - 3,472,802
  misc_feature (1)..(1): Xaa can be any naturally occurring amino acid
  MISC_FEATURE (3)..(3): 2,3-diaminopropionic acid
XLXFLSEYG

SEQ ID NO: 43 - protein, 9 aa, Artificial Sequence
  Rhodococcus erythropolis PR4 DNA nt 3,641,373 - 3,724,302
  misc_feature (1)..(1): Xaa can be any naturally occurring amino acid
  MISC_FEATURE (2)..(2): 2,3-diaminopropionic acid
XXVFTTYSS

SEQ ID NO: 44 - protein, 6 aa, Artificial Sequence
  Rhodococcus erythropolis PR4 DNA nt 3,772,211 - 3,828,404
  misc_feature (1)..(1): Xaa can be any naturally occurring amino acid
  MISC_FEATURE (3)..(3): 2,3-diaminopropionic acid
XYXGYA

SEQ ID NO: 45 - protein, 8 aa, Artificial Sequence
  Rhodococcus jostii RHA1 nt 124,521 - 171,307
  misc_feature (1)..(1): Xaa can be any naturally occurring amino acid
  MISC_FEATURE (3)..(3): 2,3-diaminopropionic acid
XYXLYTIV

SEQ ID NO: 46 - protein, 8 aa, Artificial Sequence
  Rhodococcus jostii RHA1 nt 257,548 - 324,163
  misc_feature (1)..(1): Xaa can be any naturally occurring amino acid
  MISC_FEATURE (4)..(4): 2,3-diaminopropionic acid
XWEXFTIF

SEQ ID NO: 47 - protein, 6 aa, Artificial Sequence
  Rhodococcus jostii RHA1 nt 5,421,621 - 5,460,424
  misc_feature (1)..(1): Xaa can be any naturally occurring amino acid
  MISC_FEATURE (3)..(3): 2,3-diaminopropionic acid
XFXTRF

SEQ ID NO: 48 - protein, 10 aa, Artificial Sequence
  Corallococcus coralloides DSM 2259 nt 3,158,299 - 3,187,644
  misc_feature (1)..(1): Xaa can be any naturally occurring amino acid
XKQVAIEAAV

SEQ ID NO: 49 - protein, 8 aa, Artificial Sequence
  Rhodococcus opacus B4 nt 3,545,422 - 3,611,002
  misc_feature (1)..(1): Xaa can be any naturally occurring amino acid
  MISC_FEATURE (3)..(3): 2,3-diaminopropionic acid
XLXFLSYW

SEQ ID NO: 50 - protein, 14 aa, Artificial Sequence
  Teredinibacter turnerae T7901 nt 2,500,801 - 2,571,480
  misc_feature (1)..(1): Xaa can be any naturally occurring amino acid
  MISC_FEATURE (2)..(2): ornithine
XXSVDRTGIIAAIL

SEQ ID NO: 51 - protein, 6 aa, Artificial Sequence
  Thermobifida fusca YX nt 2,152,724 - 2,214,837
  misc_feature (1)..(1): Xaa can be any naturally occurring amino acid
XKGGSD

SEQ ID NO: 52 - protein, 8 aa, Artificial Sequence
  Rhodococcus jostii RHA1 nt 5,374,757 - 5,421,456
  misc_feature (1)..(1): Xaa can be any naturally occurring amino acid
  MISC_FEATURE (3)..(3): 2,3-diaminopropionic acid
XLXYPTIF

SEQ ID NO: 53 - protein, 8 aa, Artificial Sequence
  Burkholderia rhizoxinica HKI 454 nt 1,815,672 - 1,884,887
  misc_feature (1)..(1): Xaa can be any naturally occurring amino acid
  MISC_FEATURE (3)..(3): 2,3-diaminopropionic acid
XVXVTSYY

SEQ ID NO: 54 - protein, 8 aa, Artificial Sequence
  Marinomonas mediterranea MMB-1 nt 1,028,535 - 1,099,973
  misc_feature (1)..(1): Xaa can be any naturally occurring amino acid
  MISC_FEATURE (3)..(3): 2,4-diaminobutyric acid
XDXGGKGD

SEQ ID NO: 55 - protein, 9 aa, Artificial Sequence
  Pseudomonas brassicacearum NFM421 nt 2,934,038 - 3,009,838
  misc_feature (1)..(1): Xaa can be any naturally occurring amino acid
  MISC_FEATURE (3)..(3): 2,4-diaminobutyric acid
  MISC_FEATURE (8)..(8): 2,4-diaminobutyric acid
XLXDSSDXD

SEQ ID NO: 56 - protein, 12 aa, Artificial Sequence
  Paenibacillus mucilaginosus KNP414 nt 4,432,091 - 4,514,260
  misc_feature (1)..(1): Xaa can be any naturally occurring amino acid
  MISC_FEATURE (2)..(2): ornithine
XXYVPGTIGYVD

SEQ ID NO: 57 - protein, 14 aa, Artificial Sequence
  Paenibacillus mucilaginosus KNP414 nt 3,104,571 - 3,193,674
  MISC_FEATURE (8)..(8): 2,3-diaminopropionic acid
FAFVYFVXVFVTFV

SEQ ID NO: 58 - protein, 7 aa, Artificial Sequence
  Streptosporangium roseum DSM 43021 nt 7,175,764 - 7,243,639
  misc_feature (1)..(1): Xaa can be any naturally occurring amino acid
  MISC_FEATURE (3)..(3): 2,3-diaminopropionic acid
XIXTWTV

SEQ ID NO: 59 - protein, 8 aa, Artificial Sequence
  Xenorhabdus bovienii SS-2004 nt 739,318 - 804,275
  misc_feature (1)..(1): Xaa can be any naturally occurring amino acid
  MISC_FEATURE (3)..(3): 2,3-diaminopropionic acid
XTXLLLLS

SEQ ID NO: 60 - protein, 7 aa, Artificial Sequence
  Xenorhabdus bovienii SS-2004 nt 2,095,493 - 2,159,645
  MISC_FEATURE (1)..(1): 2,3-diaminopropionic acid
XLRLKRG

SEQ ID NO: 61 - protein, 9 aa, Artificial Sequence
  Pseudomonas protegens Pf-5 nt 4,705,296 - 4,776,269
  misc_feature (1)..(1): Xaa can be any naturally occurring amino acid
  MISC_FEATURE (3)..(3): 2,3-diaminopropionic acid
  MISC_FEATURE (8)..(8): ornithine
XDXQTAAXQ

SEQ ID NO: 62 - protein, 8 aa, Artificial Sequence
  Actinosynnema mirum DSM 43827 nt 5,416,139 - 5,503,748
  misc_feature (1)..(1): Xaa can be any naturally occurring amino acid
  MISC_FEATURE (2)..(2): 2,3-diaminopropionic acid
  MISC_FEATURE (5)..(5): ornithine
XXPYXYSY

SEQ ID NO: 63 - protein, 6 aa, Artificial Sequence
  Bacillus thuringiensis plasmid BMB171 nt 151,784 - 215,426
  misc_feature (1)..(1): Xaa can be any naturally occurring amino acid
  MISC_FEATURE (2)..(2): ornithine
XXRIRL

SEQ ID NO: 64 - protein, 7 aa, Artificial Sequence
  Chromobacterium violaceum ATCC 12472 nt 3,016,595 - 3,076,834
  misc_feature (1)..(1): Xaa can be any naturally occurring amino acid
  MISC_FEATURE (2)..(2): 2,4-diaminobutyric acid
XXSVTVL

SEQ ID NO: 65 - protein, 8 aa, Artificial Sequence
  Frankia alni ACN14a nt 4,470,422 - 4,555,531
  misc_feature (1)..(1): Xaa can be any naturally occurring amino acid
  MISC_FEATURE (3)..(3): 2,3-diaminopropionic acid
  MISC_FEATURE (6)..(6): ornithine
XNXLLXAL

SEQ ID NO: 66 - protein, 8 aa, Artificial Sequence
  Paenibacillus polymyxa E681 nt 61,938 - 131,860
  misc_feature (1)..(1): Xaa can be any naturally occurring amino acid
  MISC_FEATURE (2)..(2): 2,3-diaminopropionic acid
XXTVFTNA

SEQ ID NO: 67 - protein, 8 aa, Artificial Sequence
  Paenibacillus polymyxa SC2 nt 1,071,648 - 1,147,733
  misc_feature (1)..(1): Xaa can be any naturally occurring amino acid
  MISC_FEATURE (3)..(3): 2,3-diaminopropionic acid
XYXSSSIS

SEQ ID NO: 68 - protein, 8 aa, Artificial Sequence
  Paenibacillus mucilaginosus KNP414 nt 4,920,286 - 5,002,941
  misc_feature (1)..(1): Xaa can be any naturally occurring amino acid
  MISC_FEATURE (3)..(3): ornithine
XFXVIFAF

SEQ ID NO: 69 - protein, 10 aa, Artificial Sequence
  Paenibacillus mucilaginosus 3016 nt 4,198,074 - 4,268,990
  misc_feature (1)..(1): Xaa can be any naturally occurring amino acid
  MISC_FEATURE (2)..(2): ornithine
XXYVPGYGVD

SEQ ID NO: 70 - protein, 8 aa, Artificial Sequence
  Rhodococcus equi 103S nt 4,503,597 - 4,577,253
  misc_feature (1)..(1): Xaa can be any naturally occurring amino acid
  MISC_FEATURE (3)..(3): 2,3-diaminopropionic acid
XAXALTLW

SEQ ID NO: 71 - protein, 11 aa, Artificial Sequence
  Streptomyces griseus NBRC 13350 nt 3,747,228 - 3,843,604
  misc_feature (1)..(1): Xaa can be any naturally occurring amino acid
  MISC_FEATURE (2)..(2): 2,3-diaminopropionic acid
XXNWDWSSYVV

SEQ ID NO: 72 - protein, 11 aa, Artificial Sequence
  Streptomyces violaceusniger Tu 4113 nt 10,111,293 - 10,191,037
  misc_feature (1)..(1): Xaa can be any naturally occurring amino acid
  MISC_FEATURE (2)..(2): 2,3-diaminopropionic acid
XXYTGWPWGYN

SEQ ID NO: 73 - protein, 6 aa, Artificial Sequence
  Streptomyces collinus Tu 365 nt 341,018 - 489,943
  misc_feature (1)..(1): Xaa can be any naturally occurring amino acid
XKSVAG

SEQ ID NO: 74 - protein, 8 aa, Artificial Sequence
  Xanthomonas albilineans GPE PC73 nt 836,222 - 899,513
  misc_feature (1)..(1): Xaa can be any naturally occurring amino acid
  MISC_FEATURE (4)..(4): 2,4-diaminobutyric acid
XKFXNAVQ

SEQ ID NO: 75 - protein, 15 aa, Artificial Sequence
  Bradyrhizobium sp. BTAi1 nt 7,072,456 - 7,158,452
  misc_feature (1)..(1): Xaa can be any naturally occurring amino acid
  MISC_FEATURE (3)..(3): 2,3-diaminopropionic acid
XLXAVVASLLTSSVL

SEQ ID NO: 76 - protein, 9 aa, Artificial Sequence
  Azospirillum sp. B510 pAB510a nt 962,998 - 1,053,897
  misc_feature (1)..(1): Xaa can be any naturally occurring amino acid
  MISC_FEATURE (6)..(6): 2,3-diaminopropionic acid
XADGDXSDG

SEQ ID NO: 77 - protein, 13 aa, Artificial Sequence
  Brevibacillus brevis NBRC 100599 nt 3,002,466 - 3,091,712
LVVLAVAVVVWLR

SEQ ID NO: 78 - protein, 11 aa, Artificial Sequence
  Nocardia cyriacigeorgica GUH-2 nt 5,347,951 - 5,451,235
PTWSSTSWSWS

SEQ ID NO: 79 - protein, 6 aa, Artificial Sequence
  Burkholderia sp. YI23 nt 87,366 - 144,166
  MISC_FEATURE (5)..(5): N-alpha-methylation
FASTXL

SEQ ID NO: 80 - protein, 7 aa, Artificial Sequence
  Streptomyces cattleya NRRL 8057 nt 2,784,016 - 2,856,738
DNYFYSR

SEQ ID NO: 81 - protein, 6 aa, Artificial Sequence
  Burkholderia pseudomallei K96243 nt 1,978,620 - 2,037,444
  misc_feature (1)..(1): Xaa can be any naturally occurring amino acid
  MISC_FEATURE (5)..(5): 2,4-diaminobutyric acid
XLLVXS

SEQ ID NO: 82 - protein, 9 aa, Artificial Sequence
  Bradyrhizobium sp. BTAi1 nt 1,110,518 - 1,183,121
  misc_feature (1)..(1): Xaa can be any naturally occurring amino acid
XDGFGEEGE

SEQ ID NO: 83 - protein, 7 aa, Artificial Sequence
  Myxococcus stipitatus DSM 14675 nt 7,943,455 - 8,003,631
  misc_feature (1)..(1): Xaa can be any naturally occurring amino acid
XGQKAGS

SEQ ID NO: 84 - protein, 7 aa, Artificial Sequence
  Bacillus subtilis subsp. subtilis str. 168 nt 356,968 - 422,359
  misc_feature (1)..(1): Xaa can be any naturally occurring amino acid
  MISC_FEATURE (2)..(2): 2,4-diaminobutyric acid
XXSVTVL

SEQ ID NO: 85 - protein, 10 aa, Artificial Sequence
  Corallococcus coralloides DSM 2259 nt 4,855,502 - 4,927,016
  misc_feature (1)..(1): Xaa can be any naturally occurring amino acid
XAAFVDFYNT

SEQ ID NO: 86 - protein, 9 aa, Artificial Sequence
  Myxococcus stipitatus DSM 14675 nt 5,568,702 - 5,644,506
  misc_feature (1)..(1): Xaa can be any naturally occurring amino acid
XDFYVYVNT

SEQ ID NO: 87 - protein, 7 aa, Artificial Sequence
  Myxococcus stipitatus DSM 14675 nt 7,943,455 - 8,003,631
  misc_feature (1)..(1): Xaa can be any naturally occurring amino acid
XGQVAGS

SEQ ID NO: 88 - protein, 7 aa, Artificial Sequence
  Xanthomonas oryzae pv. oryzicola BLS256 nt 2,606,212 - 2,667,348
  misc_feature (1)..(1): Xaa can be any naturally occurring amino acid
XNVLTLV

SEQ ID NO: 89 - protein, 7 aa, Artificial Sequence
  Amycolicicoccus subflavus DQS3-9A nt 4,533,351 - 4,595,796
  misc_feature (1)..(1): Xaa can be any naturally occurring amino acid
XIVFFFS

SEQ ID NO: 90 - protein, 6 aa, Artificial Sequence
  Burkholderia rhizoxinica HKI 454 nt 1,961,984 - 2,037,081
  misc_feature (1)..(1): Xaa can be any naturally occurring amino acid
XEVVEQ

SEQ ID NO: 91 - protein, 6 aa, Artificial Sequence
  Actinoplanes missouriensis 431 nt 5,221,571 - 5,289,542
  misc_feature (1)..(1): Xaa can be any naturally occurring amino acid
XVNSQQ

SEQ ID NO: 92 - protein, 6 aa, Artificial Sequence
  Bacillus subtilis subsp. subtilis str. 168 nt 5,800,513 - 5,900,006
  misc_feature (1)..(1): Xaa can be any naturally occurring amino acid
XLSLSQ

SEQ ID NO: 93 - protein, 6 aa, Artificial Sequence
  Streptomyces avermitilis MA-4680 nt 4,480,084 - 4,546,900
  misc_feature (1)..(1): Xaa can be any naturally occurring amino acid
XANTNN

SEQ ID NO: 94 - protein, 7 aa, Artificial Sequence
  Kitasatospora setae KM-6054 nt 6,477,303 - 6,546,097
  misc_feature (1)..(1): Xaa can be any naturally occurring amino acid
XNGLETF

SEQ ID NO: 95 - protein, 8 aa, Artificial Sequence
  Marinomonas mediterranea MMB-1 nt 1,028,535 - 1,099,973
  misc_feature (1)..(1): Xaa can be any naturally occurring amino acid
XDEGGDGD

SEQ ID NO: 96 - protein, 9 aa, Artificial Sequence
  SEQ ID NO 96
  misc_feature (1)..(1): Xaa can be any naturally occurring amino acid
  MISC_FEATURE (2)..(2): ornithine
  MISC_FEATURE (6)..(6): ornithine
XXIYWXLIY

SEQ ID NO: 97 - DNA, 21 bp, Artificial Sequence
  SEQ ID NO 97
catcatcatcatcatcatcat

SEQ ID NO: 98 - protein, 7 aa, Artificial Sequence
  SEQ ID NO 98
  MISC_FEATURE (1)..(1): 2,3-diaminopropionic acid
XPQAVFP

SEQ ID NO: 99 - DNA, 13 bp, Artificial Sequence
  SEQ ID NO 99
tcatcatcatcat

SEQ ID NO: 100 - protein, 4 aa, Artificial Sequence
  SEQ ID NO 100
  MISC_FEATURE (2)..(2): 2,3-diaminopropionic acid
AXSK

SEQ ID NO: 101 - DNA, 12 bp, Artificial Sequence
  SEQ ID NO 101
catcatcatcat

SEQ ID NO: 102 - protein, 4 aa, Artificial Sequence
  SEQ ID NO 102
RKGV

SEQ ID NO: 103 - protein, 9 aa, Artificial Sequence
  SEQ ID NO 103
  MISC_FEATURE (1)..(1): 2,3-diaminopropionic acid
  MISC_FEATURE (4)..(4): ornithine
  MISC_FEATURE (7)..(7): ornithine
XNLXSPXFT

SEQ ID NO: 104 - DNA, 15 bp, Artificial Sequence
  SEQ ID NO 104
catcatcatcatcat

SEQ ID NO: 105 - protein, 5 aa, Artificial Sequence
  SEQ ID NO 105
  MISC_FEATURE (1)..(1): ornithine
XRIRL

SEQ ID NO: 106 - protein, 5 aa, Artificial Sequence
  SEQ ID NO 106
WTGYR

SEQ ID NO: 107 - protein, 5 aa, Artificial Sequence
  SEQ ID NO 107
LTVSI

SEQ ID NO: 108 - protein, 5 aa, Artificial Sequence
  SEQ ID NO 108
FSTRF

SEQ ID NO: 109 - DNA, 27 bp, Artificial Sequence
  SEQ ID NO 109
catcatcatcatcatcatcatcatcat

SEQ ID NO: 110 - protein, 7 aa, Artificial Sequence
  SEQ ID NO 110
  MISC_FEATURE (2)..(2): ornithine
FXVIFAF

SEQ ID NO: 111 - DNA, 16 bp, Artificial Sequence
  SEQ ID NO 111
catcatcatcatcatc

SEQ ID NO: 112 - protein, 5 aa, Artificial Sequence
  SEQ ID NO 112
VVWLR

SEQ ID NO: 113 - DNA, 18 bp, Artificial Sequence
  SEQ ID NO 113
catcatcatcatcatcat

SEQ ID NO: 114 - protein, 6 aa, Artificial Sequence
  SEQ ID NO 114
VLAVAV

(19) United States
(12) Patent Application Publication
(10) Pub. No.: US 2023/0272010 A1
(43) Pub. Date: Aug. 31, 2023

(54) ANTIBACTERIAL SYNTHETIC-BIOINFORMATIC NATURAL PRODUCTS AND USES THEREOF

(71) Applicant: The Rockefeller University, New York, NY (US)

(72) Inventors: Sean Brady, New York, NY (US); John Chu, New York, NY (US)

(21) Appl. No.: 18/008,006

(22) PCT Filed: Jun. 4, 2021
(86) PCT No.: PCT/US21/35864

§ 371 Date: Dec. 2, 2022

Related U.S. Application Data
(60) Provisional application No. 63/035,169, filed on Jun. 5, 2020.

(51) Int. Cl. C07K 7/64; A61P 31/04

(52) U.S. Cl. CPC C07K 7/64; A61P 31/04

(57) ABSTRACT

The present invention relates to novel compounds and compositions thereof that are useful as antimicrobial agents. The present invention also relates to methods of generating said antimicrobial compounds and compositions thereof as well as methods for treating or preventing a bacterial infection using said compounds or compositions thereof. The present invention further discloses methods for preventing or reducing the growth or proliferation of microorganisms.

### CROSS-REFERENCE TO RELATED APPLICATIONS

This application claims priority to U.S. Provisional Application Ser. No. 63/035,169, filed Jun. 5, 2020, the disclosure of which is hereby incorporated by reference herein in its entirety.

### STATEMENT REGARDING FEDERALLY SPONSORED RESEARCH OR DEVELOPMENT

This invention was made with government support under NIH 1U19AI142731 and 5R35GM122559 awarded by the National Institutes of Health. The government has certain rights in the invention.

### BACKGROUND OF THE INVENTION

Many antibiotics in clinical use today are either molecules originally discovered from bacterial fermentation broths or synthetic variants of these metabolites (Newman D J et al., 2016, J. Nat. Prod., 79:629-661). Key to the tremendous success of natural products in antibiotic 20 discovery pipelines is the amazing diversity of ways by which they have evolved to inhibit bacterial growth (Walsh C T, 2003, ASM Press, Antibiotics: Actions, Origins, Resistance). However, despite the success of culture-dependent antibiotic discovery programs, large genome and metagenome sequencing efforts indicate that only a small fraction of the bacterial biosynthetic potential present in nature has been accessed (Staley J T et al., 1985, Annu. Rev. Microbiol., 39:321-346; Jensen P R, 2016, Trends Microbiol., 24:968-977; Libis V et al., 2019, Nat. Commun., 10:3848). In fact, sequenced biosynthetic gene clusters that do not appear to correspond to any known metabolites far exceed the number of bacterial natural products that have been isolated and structurally characterized (Libis V et al., 2019, Nat. Commun., 10:3848; Blin K et al., 2017, Nucleic Acids Res., 45:D555-D559). The development of new methods for converting this untapped reservoir of genetic information into chemical structures should help to populate antibiotic discovery pipelines with new molecules that function by diverse modes of action (Handelsman J et al., 1998, Chem. Biol., 5:R245-249; Charlop-Powers Z et al., 2014, Curr. Opin. Microbiol., 19:70-75; Kalkreuter E et al., 2020, Trends Pharmacol. Sci., 41:13-26).

Thus, there is a need in the art for methods that develop mechanically diverse antibacterial compounds as well as methods that use said antibacterial chemical compounds in treating or preventing various disease or disorders. The present invention addresses this unmet need in the art.

### SUMMARY OF THE INVENTION

In one aspect, the present invention provides a compound having the structure of

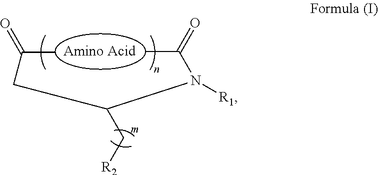

or a pharmaceutically acceptable salt, solvate, or tautomer thereof,

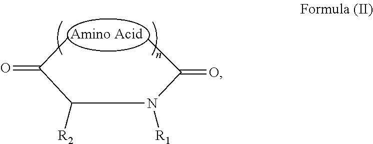

or a pharmaceutically acceptable salt, solvate, or tautomer thereof, or

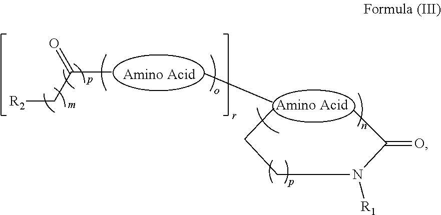

or a pharmaceutically acceptable salt, solvate, or tautomer thereof.

In various embodiments, the compound having the structure of Formula (III) is a compound having the structure of

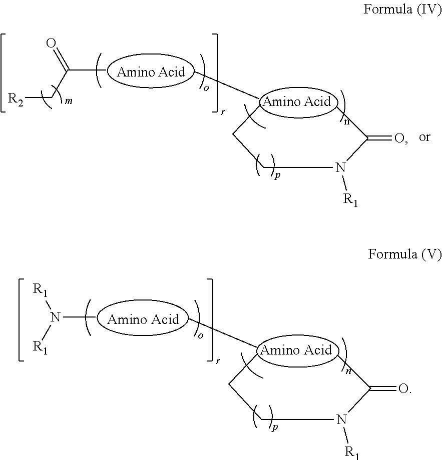

In some embodiments, the compound having the structure of Formula (I) is a compound having the structure of

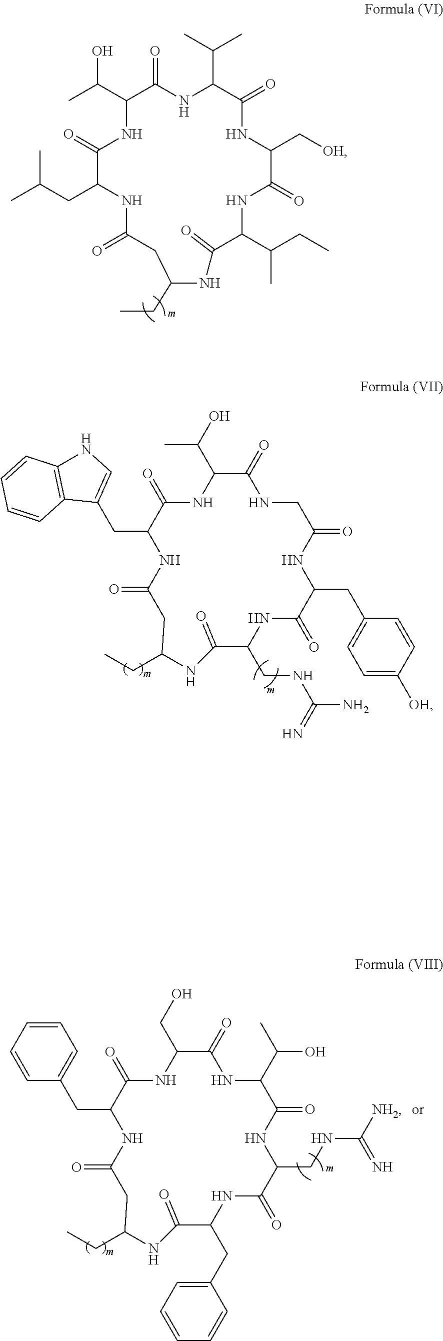

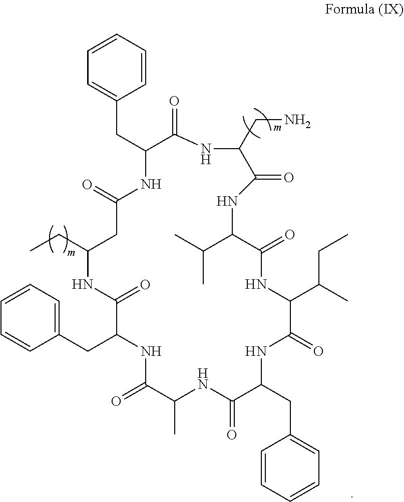

In some embodiments, the compound having the structure of Formula (II) is a compound having the structure of Formula (X)

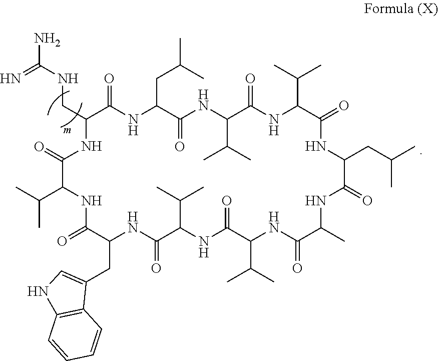

In some embodiments, the compound having the structure of Formula (III) is a compound having the structure of

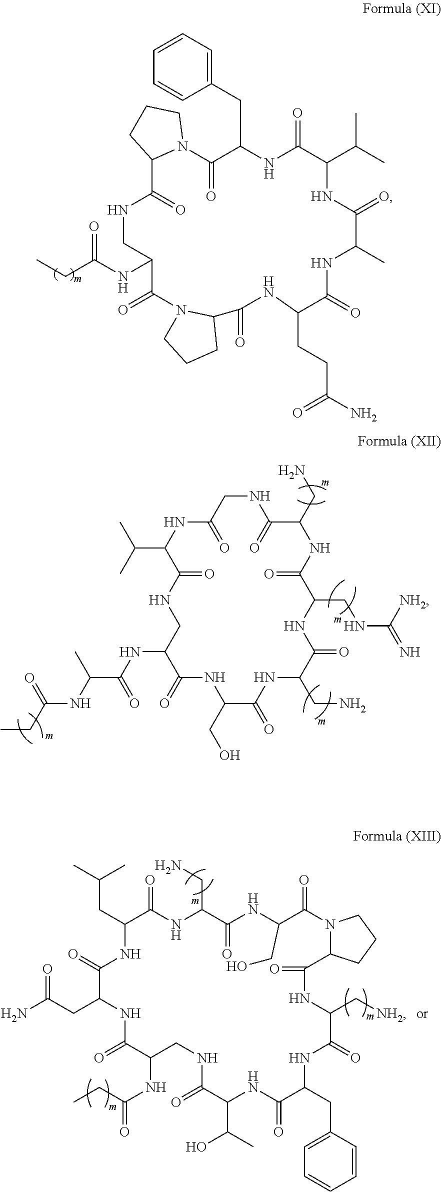

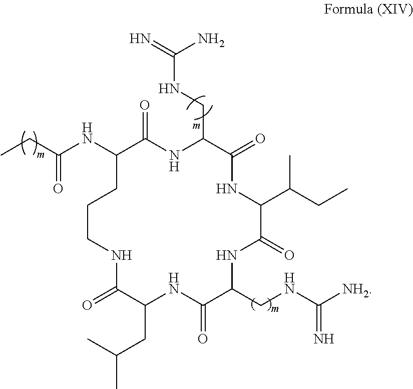

In some embodiments, each occurrence of R₁ and R₂ is independently hydrogen, hydroxyl, hydroxylalkyl, amino, aminoalkyl, carboxyl, alkyl, cycloalkyl, heterocycloalkyl, aryl, arylalkyl, heteroaryl, heteroarylalkyl, carbamate, guanidine, or guanidine alkyl. In some embodiments, each occurrence of R₁ and R₂ is independently hydrogen, hydroxylalkyl, aminoalkyl, alkyl, arylalkyl, heteroarylalkyl, or guanidine alkyl.

In some embodiments, each occurrence of m and o is independently an integer from 0 to 100. In some embodiments, each occurrence of m and o is independently an integer from 0 to 20. In some embodiments, each occurrence of m and o is independently an integer from 0 to 15. In some embodiments, each occurrence of m is independently an integer from 1 to 20.

In some embodiments, each occurrence of n and r is independently an integer from 1 to 100. In some embodiments, each occurrence of n and r is independently an integer from 1 to 20. In some embodiments, each occurrence of n and r is independently an integer from 1 to 15.

In some embodiments, each occurrence of p is independently an integer from 0 to 3. In some embodiments, each occurrence of p is independently an integer from 0 to 2.

In one embodiment, the compound inhibits the growth of at least one microorganism. In one embodiment, the compound inhibits the growth of at least one microorganism at a minimal inhibitory concentration (MIC) between around 1 μg/mL and 10,000 μg/mL. In one embodiment, the compound inhibits at least one microorganism at a MIC around 8 μg/mL. In one embodiment, the microorganism is resistant to at least one antibiotic.

In one embodiment, the compound is an antimicrobial compound.

In one embodiment, the compound is a nonribosomal peptide.

In one embodiment, the compound is a synthetic-bioinformatic natural product (syn-BNP).

In one embodiment, the compound is a syn-BNP cyclic peptide antibiotics (syCPA).

In various aspects, the present invention provides a composition comprising at least one compound of the present invention. In some embodiments, the compound reduces the growth of at least one microorganism; reduces cell wall biosynthesis; induces cell lysis; induces membrane depolarization; dysregulates at least one mitochondrial ClpP protease; or any combination thereof.

In some embodiments, the microorganism is a bacterium, virus, fungus, parasite, or any combination thereof. In some embodiments, the bacterium is Acinetobacter baumannii, Bacillus anthracis, Bacillus cereus, Bacillus subtilis, Enterobacter cloacae, Escherichia coli, Enterococcus faecium, Klebsiella pneumoniae, Lactobacillus rhamnosus, Listeria monocytogenes, Mycobacterium tuberculosis, Proteus mirabills, Pseudomonas aeruginosa, Salmonella enterica, Staphylococcus aureus, Staphylococcus epidermidis, or any combination thereof.

In various aspects, the present invention provides a method of preventing or reducing the growth or proliferation of a microorganism. In some embodiments, the method comprises contacting the microorganism with at least one compound of the present invention or a composition thereof.

In one embodiment, the microorganism is resistant to at least one antibiotic.

In various aspects, the present invention provides a method of treating or preventing a disease or disorder in a subject in need thereof. In some embodiments, the method comprises administering a therapeutically effective amount of at least one compound of the present invention or a composition thereof to the subject.

In some embodiments, the disease or disorder is a disease or disorder associated with at least one microorganism. In one embodiment, the disease or disorder associated with at least one microorganism is a bacterial infection. In some embodiments, the bacterial infection is caused by a bacterium selected from Acinetobacter baumannii, Actinomyces israelii, Bacillus anthracis, Bacillus cereus, Bacillus subtilis, Bacteroides fragilis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtherias, Ehrlichia canis, Ehrlichia chaffeensis, Enterobacter cloacae, Enterococcus faecalis, Enterococcus faecium, Escherichia coli, Francisella tularensis, Haemophilus influenzae, Helicobacter pylori, Klebsiella pneumoniae, Lactobacillus rhamnosus, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Proteus mirabills, Pseudomonas aeruginosa, Nocardia asteroids, Rickettsia rickettsia, Salmonella enterica, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Candida albicans, Candida tropicalis, Candida glabrata, Candida parapsilosis, Candida krusei, Candida lusitaniae, Candida kefyr, Candida guilliermondii, and Candida dubliniensis, Yersinia pestis, Yersinia enterocolitica, Yersinia pseudotuberculosis, or any combination thereof. In some embodiments, the bacterial infection is caused by a bacterium selected from Acinetobacter baumannii, Bacillus anthracis, Bacillus cereus, Bacillus subtilis, Enterobacter cloacae, Escherichia coli, Enterococcus faecium, Klebsiella pneumoniae, Lactobacillus rhamnosus, Listeria monocytogenes, Mycobacterium tuberculosis, Proteus mirabills, Pseudomonas aeruginosa, Salmonella enterica, Staphylococcus aureus, Staphylococcus epidermidis, or any combination thereof.

In one embodiment, the disease or disorder is a cancer.

In one aspect, the present invention provides a method of preparing a syn-BNP. In some embodiments, the method comprises: a) analyzing nonribosomal peptide synthase (NRPS) gene clusters, wherein the NRPS gene clusters encode one or more peptides; b) identifying one or more peptides that are encoded by the NRPS gene clusters, wherein the peptides comprise at least 4 amino acids; c) synthesizing the peptides; and d) covalently cyclizing the peptides to generate at least one syn-BNP.

In another aspect, the present invention provides a method of preparing a syCPA. In some embodiments, the method comprises: a) analyzing NRPS gene clusters, wherein the NRPS gene clusters encode one or more peptides; b) identifying one or more peptides that are encoded by the NRPS gene clusters, wherein the peptides comprise at least 4 amino acids; c) synthesizing the peptides; d) covalently cyclizing the peptides to generate at least one syn-BNP; e) exposing the syn-BNP to at least one bacterium; and f) identifying the syn-BNP that reduces the level of at least one bacterium.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1, comprising FIG. 1A through FIG. 1C, depicts a schematic representation of the development of various synthetic-Bioinformatic Natural Products (syn-BNPs). FIG. 1A depicts a schematic representation of enzymatic machinery in biological systems that produced a nonribosomal peptide (NRP) based on the information encoded within biosynthetic genes. In a syn-BNP approach, chemical synthesis produced a close structural mimic based on predictions made by bioinformatic algorithms. FIG. 1B depicts the devised synthetic scheme that allowed each predicted peptide sequence to be cyclized in two ways at the desired position. FIG. 1C depicts a schematic representation of the development of syn-BNPs with diverse modes of action.

FIG. 2, comprising FIG. 2A through FIG. 2C, depicts a schematic representation of syn-BNPs screen against a panel of bacteria to identify new antibiotics. FIG. 2A depicts a schematic representation of syn-BNPs being screened against a panel of bacteria to identify new antibiotics. This panel included model Gram-positive and Gram-negative bacteria (B. subtilis and E. coli, respectively) as well as the ESKAPE pathogens. FIG. 2B depicts representative Syn-BNP that was given a semi-quantitative score for its activity against each bacterium that ranged from 0 (inactive) to 3 (most potent) based on the size of its zone of growth inhibition. FIG. 2C depicts representative 171 Syn-BNP cyclic peptides that were synthesized based on bioinformatic predictions of 96 nonribosomal peptide synthetase (NRPS) gene clusters. Fifteen primary hits were identified from this Syn-BNP collection. Nine hits were validated upon re-synthesis.

FIG. 3 depicts representative structures of validated syn-BNP cyclic peptide antibiotics (SyCPAs) and representative key features of the NRPS gene cluster from which each was predicted. Bold bonds indicate the site of cyclization.

FIG. 4, comprising FIG. 4A through FIG. 4D, depicts representative minimum inhibitory concentrations (MICs) and cytotoxicity of representative SyCPAs. FIG. 4A depicts representative MICs of the SyCPAs (μg/mL, “>” indicates the MIC is greater than 64 μg/mL; the highest concentration tested for M. tuberculosis was 12.5 μg/mL). FIG. 4B depicts representative results demonstrating cytotoxicity of B. subtilis and S. aureus active SyCPAs were assessed using HeLa cells and the MTT metabolic activity assay. Cell survival was normalized to that of the DMSO control. FIG. 4C depicts representative results from Syn-BNP antibiotics that were assayed at 4×MIC against B. subtilis. FIG. 4D depicts representative results from Syn-BNP antibiotics that were assayed at 4×MIC against S. aureus. Two fluorescent dyes were used to probe potential membrane acting mechanisms—SYTOX Green for lysis and DiSC₃(5) for depolarization. DMSO was used as the negative control in all assays (light gray traces).

FIG. 5, comprising FIG. 5A through FIG. 51, depicts representative SyCPAs designed based on biosynthetic gene clusters that have not been previously associated with any natural products and their structures did not closely resemble any natural products deposited in the Dictionary of Natural Products or SciFinder databases. FIG. 5A depicts representative SyCPA 2 (Burkholderia gladioli BSR3, 1,312,845-1,376,223 nt). FIG. 5B depicts representative SyCPA 4 (Burkholderia gladioli BSR3, 2,103,056-2,186,245 nt). FIG. 5C depicts representative SyCPA 12 (Dickeya dadantii Ech586, 3,414,889-3,485,467 nt). FIG. 5D depicts representative SyCPA 63 (Bacillus thuringiensis BMB171 plasmid, 151,784-215,426 nt). FIG. 5E depicts representative SyCPA 102 (Rhodococcus opacus B4, 5,997386-6,053,147 nt). FIG. 5F depicts representative SyCPA 116 (Collimonas fungivorans Ter331, 291,223-348,100 nt). FIG. 5G depicts representative SyCPA 123 (Rhodococcus jostii RHA1, 5,421,621-5,460,424 nt). Figure SI depicts representative SyCPA 144 (Paenibacillus mucilaginosus KNP414, 4,920,286-5,002,941 nt). Figure SI depicts representative SyCPA 153 (Brevibacillus brevis NBRC 100599, 3,002,466-3,091,712 nt).

FIG. 6, comprising FIG. 6A through FIG. 6D, depicts representative peptides that were tested for inducing the accumulation of the lipid II biosynthetic precursor UDP-MurNAc-pentapeptide as well as the improved activities of SyCPAs when tested in combination with polymyxin. FIG. 6A depicts representative peptides that were tested at 20 μg/mL for inducing the accumulation of the lipid II biosynthetic precursor UDP-MurNAc-pentapeptide. Vancomycin and DMSO were used as the positive and negative controls, respectively (dark and light grey traces). FIG. 6B depicts representative results demonstrating that SyCPA 4 (gladiosyn) was most active against Gram-positive Bacilli. FIG. 6C depicts representative results demonstrating that Bacillus and Gram-negative bacteria used a meso-diaminopimelic acid to crosslink their peptidoglycans in cell wall biosynthesis, as opposed to using lysine in those of other Gram-positive bacteria. FIG. 6D depicts representative results demonstrating that SyCPAs showed improved activities when tested in combination with polymyxin at ¼×MIC of each respective bacterium (μg/mL, “>” indicates the MIC is greater than 64 μg/mL).

FIG. 7, comprising FIG. 7A through 7C, depicts representative mutations in clpP that conferred resistance to SyCPA 116, representative results for the dysregulation of ClpP, and schematic comparison of SyCPA 116 (collimosyn) and ADEP structures. FIG. 7A depicts representative mutations in clpP that conferred resistance to SyCPA 116 (collimosyn). FIG. 7B depicts representative results demonstrating that dysregulation of ClpP resulted in uncontrolled protein degradation which is lethal to S. aureus. FIG. 7C depicts schematic comparison of SyCPA 116 (collimosyn) and ADEP structures.

### DETAILED DESCRIPTION

The present invention relates, in part, to novel compounds and compositions thereof that are useful as antimicrobial agents. In one aspect, the present invention also relates, in part, to methods of generating said antimicrobial compounds and compositions thereof. In another aspect, the present invention relates, in part, to methods of treating or preventing a various diseases or disorders using said compounds or compositions thereof. In one embodiment, the present invention relates to methods of treating or preventing a bacterial infection using said antimicrobial compounds or compositions thereof. In another aspect, the present invention further relates, in part, to methods of preventing or reducing the growth or proliferation of microorganisms using said antimicrobial compounds or compositions thereof.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

“Molecule” refers to a collection of chemically bound atoms with a characteristic composition. As used herein, a molecule can be neutral or can be electrically charged. The term molecule includes biomolecules, which are molecules that are produced by an organism or are important to a living organism, including, but not limited to, proteins, peptides, lipids, DNA molecules, RNA molecules, oligonucleotides, carbohydrates, polysaccharides, glycoproteins, lipoproteins, sugars and derivatives, variants and complexes of these, including labeled analogs of these having one or more vibrational tag. The term molecule also includes candidate molecules, which comprise any molecule that it is useful, beneficial or desirable to probe its capable to interact with a molecule such as a target molecule. Candidate molecules include therapeutic candidate molecules which are molecules that may have some effect on a biological process or series of biological processes when administered. Therapeutic candidate molecules include, but are not limited to, drugs, pharmaceuticals, metabolites, potential drug candidates and metabolites of drugs, biological therapeutics, potential biological therapeutic candidates and metabolites of biological therapeutics, organic, inorganic and/or hybrid organic-inorganic molecules that interact with one or more biomolecules, molecules that inhibit, decrease or increase the bioactivity of a biomolecule, inhibitors, ligands and derivatives, variants and complexes of these. The term molecule also includes target molecules, which comprise any molecule that it is useful, beneficial or desirable to probe its capable to interact with a molecule such as a candidate molecule. Target molecules useful for identifying, characterizing and/or optimizing therapeutics and therapeutic candidates comprise biomolecules, and derivatives, variants and complexes of biomolecules. The term molecule also includes competitive binding reference molecules. Competitive binding reference molecules useful in the present invention are molecules that are known to bind, at least to some extent, to a target molecule, and in some embodiments comprise a known drug, biological therapeutic, biomolecule, lead compound in a drug discovery program, and derivatives, variants, metabolites and complexes of these.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein “polymorph” refers to crystalline forms having the same chemical composition but different spatial arrangements of the molecules, atoms, and/or ions forming the crystal.

As used herein, the term “alkyl,” or “alkyl group” by itself or as part of another substituent means, unless otherwise stated, a straight or branched chain hydrocarbon having from 1 to 12 carbon atoms. In some embodiments, the alkyl is a C₁-C₁₂ alkyl, a C₁-C₁₀ alkyl, a C₁-C₈ alkyl, a C₁-C₆ alkyl, a C₁-C₄ alkyl, or a C₁-C₃ alkyl. For example, an alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈ alkyl. A C₁-C₈ alkyl includes C₅ alkyls, C₄ alkyls, C₃ alkyls, C₂ alkyls and C₁ alkyl (i.e., methyl). A C₁-C₆ alkyl includes all moieties described above for C₁-C₈ alkyls but also includes C₆ alkyls. A C₁-C₁₀ alkyl includes all moieties described above for C₁-C₈ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples include methyl, ethyl, n-propyl, isopropyl, n-butyl, isobutyl, sec-butyl, tert-butyl, n-pentyl, neopentyl, n-hexyl, n-heptyl, n-octyl, n-nonyl, n-decyl, n-undecyl, n-dodecyl, and cyclopropylmethyl. Unless stated otherwise specifically in the specification, an alkyl group can be optionally substituted.

“Alkylene” or “alkylene chain” refers to a fully saturated, straight or branched divalent hydrocarbon, and having from one to twelve carbon atoms, and which has two points of attachment to the rest of the molecule. In some embodiments, the alkylene is a C₁-C₁₂ alkylene, a C₁-C₁₀ alkylene, a C₁-C₈ alkylene, a C₁-C₆ alkylene, a C₁-C₄ alkylene, or a C₁-C₃ alkylene. Non-limiting examples of C₁-C₁₂ alkylene include methylene, ethylene, propylene, n-butylene, ethenylene, propenylene, n-butenylene, propynylene, n-butynylene, and the like. The points of attachment of the alkylene chain to the rest of the molecule can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms, and having one or more carbon-carbon double bonds. Each alkenyl group is attached to the rest of the molecule by a single bond. Alkenyl group comprising any number of carbon atoms from 2 to 12 are included. In some embodiments, the alkenyl is a C₂-C₁₂ alkenyl, a C₂-C₁₀ alkenyl, a C₂-C₈ alkenyl, a C₂-C₆ alkenyl, a C₂-C₄ alkenyl, or a C₂-C₃ alkenyl. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. In some embodiments, the alkynyl is a C₂-C₁₂ alkynyl, a C₂-C₁₀ alkynyl, a C₂-C₈ alkynyl, a C₂-C₆ alkynyl, a C₂-C₄ alkynyl, or a C₂-C₃ alkynyl. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

The term “hydroxy” or “hydroxyl” refers to a group of the formula —OH group.

As used herein, the term “alkoxy” employed alone or in combination with other terms means, unless otherwise stated, refers to a group of the formula —ORₐ where Rₐ is an alkyl, alkenyl or alknyl group having from 1 to 12 carbon atoms, as defined above, connected to the rest of the molecule via an oxygen atom, such as, for example, methoxy, ethoxy, 1-propoxy, 2-propoxy (isopropoxy) and the higher homologs and isomers. Unless stated otherwise specifically in the specification, an alkoxy group can be optionally substituted.

As used herein, the term “halo” or “halogen” alone or as part of another substituent means, unless otherwise stated, a fluorine, chlorine, bromine, or iodine group.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

As used herein, the term “heteroalkyl” by itself or in combination with another term means, unless otherwise stated, a stable straight or branched chain alkyl group consisting of from 1 to 12 carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃. Unless stated otherwise specifically in the specification, an heteroalkyl group can be optionally substituted.

The term “amino” refers to a group of the formula —NRₐRₐ, —NHRₐ, or —NH₂, where each Rₐ is, independently, an alkyl, alkenyl or alkynyl group as defined above containing one to twelve carbon atoms. Unless stated otherwise specifically in the specification, an alkylamino group can be optionally substituted.

“Alkylamino” refers to a group of the formula —NHRₐ or —NRₐRₐ where each Rₐ is, independently, an alkyl, alkenyl or alkynyl group as defined above containing one to twelve carbon atoms. Unless stated otherwise specifically in the specification, an alkylamino group can be optionally substituted.

The term “cyano” refers to a group of the formula —CN group.

The term “imino” refers to a group of the formula ═NH group.

The term “nitro” refers to a group of the formula —NO₂ group.

The term “oxo” refers to a group of the formula the ═O group.

“Alkylcarbonyl” refers to the —C(═O)Rₐ moiety, wherein Rₐ is an alkyl, alkenyl or alkynyl group as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl group as defined above. Unless stated otherwise specifically in the specification, an alkyl carbonyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl, cycloalkenyl and cycloalkynyl as defined herein. Unless stated otherwise specifically in the specification, a carbocyclyl group can be optionally substituted.

As used herein, the term “cycloalkyl” refers to a stable mono cyclic or polycyclic non-aromatic group, wherein each of the atoms forming the ring (i.e. skeletal atoms) is a carbon atom, which can include fused or bridged ring systems, having from three to twenty carbon atoms (e.g., having from three to ten carbon atoms) and which is attached to the rest of the molecule by a single bond. In one embodiment, the cycloalkyl group is saturated or partially unsaturated. In another embodiment, the cycloalkyl group is fused with an aromatic ring. Cycloalkyl groups include groups having from 3 to 20 carbon ring atoms. Illustrative examples of cycloalkyl groups include, but are not limited to, the following moieties:

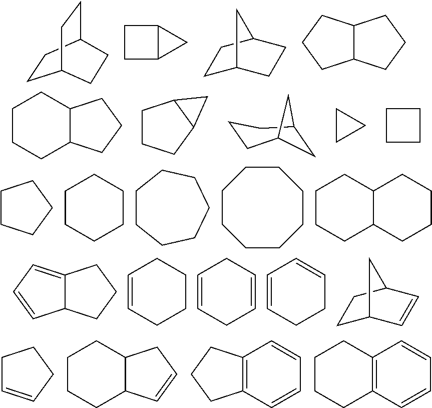

wherein any hydrogen atom in the above groups may be replaced by a bond to the molecule.

Monocyclic cycloalkyls include, but are not limited to, cyclopropyl, cyclobutyl, cyclopentyl, cyclohexyl, cycloheptyl, and cyclooctyl. Dicyclic or polycyclic cycloalkyls include, but are not limited to, tetrahydronaphthyl, indanyl, and tetrahydropentalenyl, adamantyl and norbornyl. The term cycloalkyl includes “unsaturated nonaromatic carbocyclyl,” “carbocyclyl,” “carbocyclic ring,” “carbocycle,” or “nonaromatic unsaturated carbocyclyl” groups, both of which refer to a nonaromatic carbocycle as defined herein, which contains at least one carbon double bond or one carbon triple bond.

“Cycloalkenyl” refers to a stable non aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyls include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

The terms “heterocyclic ring”, “heterocycle” and “heterocyclyl” are used interchangeably herein to refer to a 3- to 20-membered containing one to six heteroatoms each independently selected from the group consisting of O, S and N. In one embodiment, each heterocyclyl group has from 4- to 10-atoms in its ring system, and from one to three heteroatoms each independently selected from the group consisting of O, S and N. Unless stated otherwise specifically in the specification, the heterocyclyl can be a monocyclic, bicyclic, tricyclic or tetracyclic ring system, which can include fused or bridged ring systems. In one embodiment, the nitrogen, carbon, or sulfur heteroatoms may be optionally oxidized, and the nitrogen atom may be optionally quaternized. The heterocyclic system may be attached, unless otherwise stated, at any heteroatom or carbon atom that affords a stable structure. The heterocyclyl can be partially or fully saturated. A heterocycle may be polycyclic, wherein the polycyclic ring may be non-aromatic or contain both aromatic and non-aromatic rings. Unless stated otherwise specifically in the specification, a heterocyclyl group can be optionally substituted.

Examples of such heterocyclyls include, but are not limited to, aziridinyl, azetidinyl, beta lactamyl, dioxolanyl, oxazolidinyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrrolinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, oxiranyl, thiiranyl, oxetanyl, thietanyl, sulfolanyl, 2,3-dihydrofuranyl, 2,5-dihydrofuranyl, thiophanyl, 1,2,3,6-tetrahydropyridinyl, 1,4-dihydropyridinyl, piperazinyl, thiomorpholinyl, pyranyl, 2,3-dihydropyranyl, tetrahydropyranyl, 1,4-dioxanyl, 1,3-dioxanyl, homopiperazinyl, homopiperidinyl, 1,3-dioxepanyl, 4,7-dihydro-1,3-dioxepinyl, and hexamethyleneoxidyl.

Other non-limiting examples of heterocyclyl groups are:

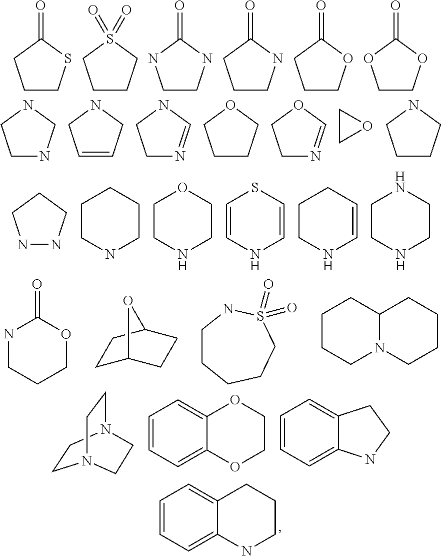

wherein any hydrogen atom in the above groups may be replaced by a bond to the molecule.

“Heterocycloalkyl” refers to a radical of the formula —R_{b}—Rₑ where R_{b} is an alkylene, alkenylene, or alkynylene group as defined above and Rₑ is a heterocyclyl radical as defined above. Unless stated otherwise specifically in the specification, a heterocycloalkylalkyl group can be optionally substituted.

“Thioalkyl” refers to a formula —SRₐ where Rₐ is an alkyl, alkenyl, or alkynyl as defined above containing one to twelve carbon atoms. Unless stated otherwise specifically in the specification, a thioalkyl group can be optionally substituted.

The term “thioxo” refers to a group of the formula the ═S group.

As used herein, the term “aromatic” refers to a carbocyclyl or heterocyclyl with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a hydrocarbon ring system, comprising hydrogen, 6 to 18 carbon atoms and at least one aromatic ring. For purposes of this disclosure, the aryl can be a monocyclic, bicyclic, tricyclic or tetracyclic ring system, which can include fused or bridged ring systems. For example, aryls include, but are not limited to, a biphenyl, or may be fused, such as naphthalene. Examples of aryl groups include benzyl, indacenyl, pyrenyl, triphenyl, phenyl, anthracyl, and naphthyl. Unless stated otherwise specifically in the specification, the term “aryl” is meant to include aryl groups that are optionally substituted.

As used herein, the term “heteroaryl” or “heteroaromatic” refers to a 5 to 20 membered ring system comprising hydrogen atoms, one to fourteen carbon atoms, one to six heteroatoms selected from the group consisting of nitrogen, oxygen and sulfur, and at least one aromatic ring. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. A polycyclic heteroaryl may include one or more rings that are partially saturated. Examples include the following moieties:

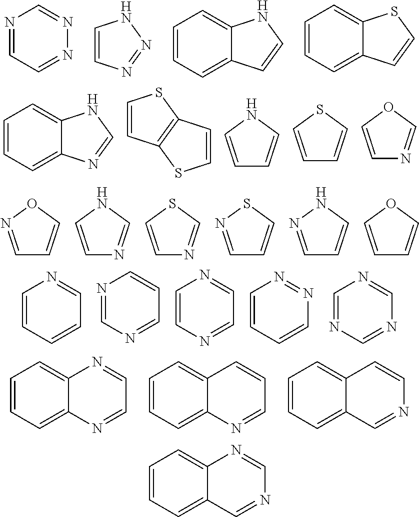

wherein any hydrogen atom in the above groups may be replaced by a bond to the molecule.

Examples of heteroaryl groups include, but are not limited to, pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, 1,3,4-oxadiazolyl, indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl. Unless stated otherwise specifically in the specification, a heteroaryl group can be optionally substituted.

“Aralkyl” or “arylalkyl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

Aralkenyl” or “arylalkenyl” refers to a radical of the formula —R_{b}—R_{c} where R_{b} is an alkenylene o group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

As used herein, the term “substituted” means any of the above groups (i.e., alkyl, alkylene, alkenyl, alkynyl, alkoxy, aryl, carbocyclyl, cycloalkyl, cycloalkenyl, cycloalkynyl, heterocyclyl, and/or heteroaryl) wherein at least hydrogen atom is replaced by a bond to a non-hydrogen atom or group of atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with, for example, —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with, for example, —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently selected from any of the above groups, including but not limited to: hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to any of the above groups, including but not limited to amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group.

In addition, each of the foregoing substituents can also be optionally substituted with one or more of the above substituents.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

As used herein, the term “antimicrobial” refers to an ability to kill or inhibit the growth of microorganisms, including but not limited to bacteria, viruses, yeast, fungi, and protozoa, or to attenuate the severity of a microbial infection. The antimicrobial compounds or compositions of the present disclosure are compounds or compositions that may be used for cleaning or sterilization, or may be used in the treatment of disease and infection. The applications may include both in vitro and in vivo antimicrobial uses. “Applying” an antimicrobial composition may include administrating a composition into a human or animal subject.

The term “assessing” includes any form of measurement, and includes determining if an element is present or not. The terms “determining,” “measuring,” “evaluating,” “assessing” and “assaying” are used interchangeably and may include quantitative and/or qualitative determinations. Assessing may be relative or absolute. “Assessing binding” includes determining the amount of binding, and/or determining whether binding has occurred (i.e., whether binding is present or absent). “Assessing activity” includes determining the amount of activity, and/or determining whether an activity has occurred (i.e., whether an activity is present or absent).

The term “binding” refers to a direct association between at least two molecules, due to, for example, covalent, electrostatic, hydrophobic, ionic and/or hydrogen-bond interactions.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components. Alternatively, molecules or components thereof may be contacted combining a fluid component with molecules immobilized on or in a cell or on or in a substrate, such as a polymer bead, a membrane, a polymeric glass substrate or substrate surface derivatized to provide immobilization of target molecules, candidate molecules, competitive binding reference molecules or any combination of these. Molecules or components thereof may be contacted by selectively adjusting solution conditions such as, the composition of the solution, ion strength, pH or temperature. Molecules or components thereof may be contacted in a static vessel, such as a microwell of a microarray system, or a flow-through system, such as a microfluidic or nanofluidic system. Molecules or components thereof may be contacted in or on a variety of cells, media, liquids, solutions, colloids, suspensions, emulsions, gels, solids, membrane surfaces, glass surfaces, polymer surfaces, vesicle samples, bilayer samples, micelle samples and other types of cellular models or any combination of these. As used herein, the term “contacting” includes, but is not limited to, impregnating, compounding, mixing, integrating, coating, rubbing, painting, spraying, immersing, rolling, smearing and dipping.

The term “bacterium”, as used herein, refers to “pathogenic bacterium” and/or “non-pathogenic bacterium”. For example, the term “non-pathogenic bacterium” refers to bacterium that is not capable of causing disease or harmful responses in a host. In some embodiments, bacteria are commensal bacteria. Examples of bacteria include, but are not limited to Escherichia coli LF82, Enterococcus faecalis, Lactobacillis plantarum, Faecalibacterium prauznitzii, Bifidobacterium longum, Bacteroides vulgatus, Ruminococcus gnavus, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium in/antis, Bifidobacterium lactis, Bifidobacterium longum, Clostridium butyricum, Enterococcus/aecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). In some embodiments, naturally pathogenic bacteria may be genetically engineered to provide reduce or eliminate pathogenicity.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human or a non-human mammal. Non-human mammals include, for example, livestock and pets, such as ovine, bovine, porcine, canine, feline and murine mammals.

As used herein, the term “treatment” or “treating,” is defined as one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition. In one embodiment, “treatment” or “treating,” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of medicine or pharmacology. In one embodiment, the condition is selected from the group consisting of a bacterial infection, fungal infection, mycobacterial infection, viral infection, and a combination thereof.

As used herein, “treating a disease or disorder” means reducing the frequency with which a sign or symptom of the disease or disorder is experienced by a subject.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

A disease or disorder is “alleviated” if the severity of a symptom of the disease or disorder, the frequency with which such a symptom is experienced by a subject, or both, is reduced.

A “therapeutic” treatment is a treatment administered to a subject who exhibits signs or symptoms of pathology, for the purpose of diminishing or eliminating those signs or symptoms.

As used herein, the term “pharmaceutical composition” refers to a mixture of at least one compound useful within the disclosure with a pharmaceutically acceptable carrier. The pharmaceutical composition facilitates administration of the compound to a patient or subject, or use of the compound within the methods of the disclosure. Multiple techniques of administering a compound exist in the art including, but not limited to, intravenous, oral, aerosol, parenteral, ophthalmic, pulmonary and topical administration.

The phrase “effective amount”, “pharmaceutically effective amount”, or “therapeutically effective amount,” as used herein, refers to an amount that is sufficient or effective to provide the desired biological and/or clinical result (e.g., prevent, treat, delay the onset of, prevent the onset of, prevent the progression of, inhibit, decrease, or reverse a disease or disorder). That result may be reduction and/or alleviation of the signs, symptoms, or causes of a disease, or any other desired alteration of a biological system. An appropriate therapeutic amount in any individual case may be determined by one of ordinary skill in the art using routine experimentation. An “effective amount” or “therapeutically effective amount” of a compound is that amount of a compound which is sufficient to provide a beneficial effect to the subject to which the compound is administered. An “effective amount” will vary depending on the active ingredient, the state, disorder, or condition to be treated and its severity, and the age, weight, physical condition and responsiveness of the mammal to be treated.

As used herein, an “inhibitory-effective amount” is an amount that results in a detectable (e.g., measurable) amount of inhibition of an activity. In some instance, the activity is its ability to bind with another component.

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

The term “interact” or “interaction” refers to a measurable chemical or physical interaction between two components, such as a target molecule and a candidate molecule, that is capable of affecting the structure and/or composition of at least one of the components, such as a target molecule, a candidate molecule or both such that the biological activity of at least one of the components, such as the target molecule, the candidate molecule or both, is affected. Interactions capable of affecting the structure and/or composition of a component include, but are not limited to, reactions resulting in the formation of one or more covalent bonds, resulting in the breaking of one or more covalent bonds, electrostatic associations and repulsions, formation and/or disruption of hydrogen bonds, formation and/or disruption of electrostatic forces such as dipole-dipole interactions, formation and/or disruption of van der Waals interactions or processes comprising combinations of these.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” embraces addition salts of free acids or free bases. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the disclosure include, for example, metallic salts including alkali metal, alkaline earth metal and transition metal salts such as, for example, lithium, calcium, magnesium, potassium, ammonium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the term “organic solvent” refers to solvents including, but not limited to, alcohols (e.g., methanol and ethanol), ketones (e.g., acetone and methylethylketone), ethers (e.g., tetrahydrofuran), aldehydes (e.g., formaldehyde), acetonitrile, carboxylic acids (e.g., formic acid and acetic acid), methylene chloride, chloroform, alkyl carbonates, and hydrocarbons (e.g., hexane and heptane, and xylene), esters (e.g., ethyl acetate, propyl acetate, butyl acetate, amyl acetate, and combination thereof) or similar solvents.

All weight percentages (i.e., “% by weight” and “wt. %” and “w/w”) referenced herein, unless otherwise indicated, are measured relative to the total weight of the pharmaceutical composition.

As used herein, the term “potency” refers to the dose needed to produce half the maximal response (ED₅₀).

As used herein, the term “efficacy” refers to the maximal effect (Eₘₐₓ) achieved within an assay.

As used herein, the term “minimum inhibitory concentration (MIC)” refers to the lowest concentration of an antimicrobial agent that will inhibit the visible growth of a microorganism after overnight incubation. MIC values against bacteria may be determined by standard methods. See also P. A. Wayne, Methods for Dilution Antimicrobial Tests for Bacteria that Grow Aerobically; Approved Standard, Ninth Edition, 2012, CLSI Document M07-A9, Vol. 32 No. 2, which is incorporated by reference herein in its entirety.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), enzymatic labels (e.g., horseradish peroxidase, beta-galactosidase, luciferase, alkaline phosphatase), biotinyl groups, predetermined polypeptide epitopes recognized by a secondary reporter (e.g., leucine zipper pair sequences, binding sites for secondary antibodies, metal binding domains, epitope tags).

As used herein, “substantially pure” means an object species is the predominant species present (i.e., it is the most abundant of any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the terms “amino acid”, “amino acidic monomer”, or “amino acid residue” refer to any of the twenty naturally occurring amino acids including synthetic amino acids with unnatural side chains and including both D and L optical isomers.

As used herein, the terms “natural amino acid”, “naturally encoded amino acid”, “naturally occurring amino acid”, and “genetically encoded amino acid” refer to an amino acid that is one of the twenty common amino acids or pyrolysine or selenocysteine. The term “natural amino acid” includes, but is not limited to, proteinogenic amino acids.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

As used herein, the term “identical” refers to two or more sequences or subsequences which are the same.

In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

“Variant” as the term is used herein, is a molecule that differs in structure from a reference molecule, but retains essential physical or biological properties of the reference molecule. “Variant” as the term is used herein, is also a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

A “fragment” of a nucleic acid sequence that encodes an antigen may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antigen and additionally optionally comprise sequence encoding an N terminal methionine or heterologous signal peptide which is not included when calculating percent identity.

“Screening” referred to in the present invention includes not only so-called first screening for identifying a compound of the present invention among a plurality of candidate compounds, but also a counter screen for identifying a compound of the present invention among a plurality of candidate compounds.

“Test agents” or otherwise “test compounds” as used herein refers to an agent or compound that is to be screened in one or more of the assays described herein. Test agents include compounds of a variety of general types including, but not limited to, small organic molecules, known pharmaceuticals, polypeptides, carbohydrates (such as oligosaccharides and polysaccharides), polynucleotides, lipids, phospholipids, fatty acids, steroids, peptides, amino acids, or amino acid analogs. Test agents can be obtained from microbial culture supernatants or microbial culture lysates. Test agents can also be obtained from libraries, such as natural product libraries and combinatorial libraries. In addition, methods of automating assays are known that permit screening of several thousands of compounds in a short period.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates, in part, to novel compounds and compositions thereof that are useful as antimicrobial agents. In one aspect, the present invention also relates, in part, to methods of generating said antimicrobial compounds and compositions thereof. In another aspect, the present invention relates, in part, to methods of treating or preventing a various diseases or disorders using said compounds or compositions thereof. In one embodiment, the present invention relates to methods of treating or preventing a bacterial infection using said antimicrobial compounds or compositions thereof. In another aspect, the present invention further relates, in part, to methods of preventing or reducing the growth or proliferation of microorganisms using said antimicrobial compounds or compositions thereof.

### Compounds

In various aspects, the present invention provides, in part, a compound having the structure of

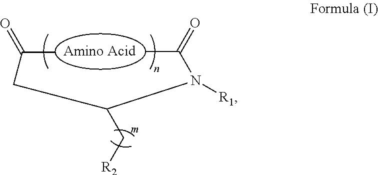

or a pharmaceutically acceptable salt, solvate, or tautomer thereof,

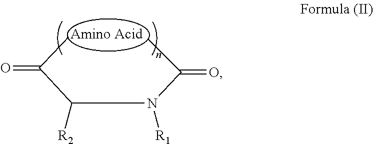

or a pharmaceutically acceptable salt, solvate, or tautomer thereof, or

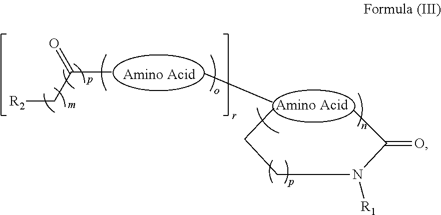

or a pharmaceutically acceptable salt, solvate, or tautomer thereof.

In some embodiments, the compound having the structure of Formula (III) is a compound having the structure of

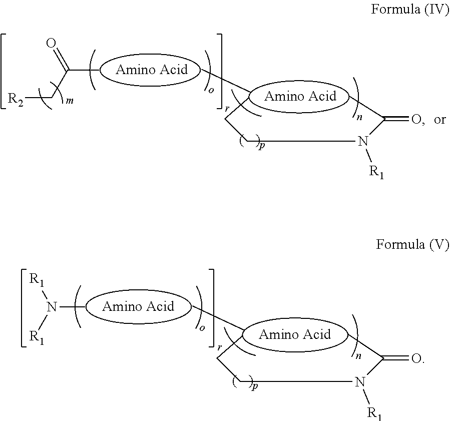

In various embodiments, each occurrence of R₁ is independently selected from hydrogen, halogen, hydroxyl, hydroxylalkyl, alkoxy, amino, aminoalkyl, carboxyl, alkyl, cycloalkyl, heterocycloalkyl, alkenyl, cycloalkenyl, heterocycloalkenyl, alkynyl, cycloalkynyl, heterocycloalkynyl, aryl, arylalkyl, heteroaryl, heteroarylalkyl, arylalkenyl, heteroarylalkenyl, arylalkynyl, heteroarylalkynyl, carbamate, guanidine, or guanidine alkyl.

In various embodiments, each occurrence of R₂ is independently selected from hydrogen, halogen, hydroxyl, hydroxylalkyl, alkoxy, amino, aminoalkyl, carboxyl, alkyl, cycloalkyl, heterocycloalkyl, alkenyl, cycloalkenyl, heterocycloalkenyl, alkynyl, cycloalkynyl, heterocycloalkynyl, aryl, arylalkyl, heteroaryl, heteroarylalkyl, arylalkenyl, heteroarylalkenyl, arylalkynyl, heteroarylalkynyl, carbamate, guanidine, or guanidine alkyl.

In various embodiments, each occurrence of m is independently an integer from 0 to 100. In some embodiments, each occurrence of m is independently an integer from 0 to 50. In some embodiments, each occurrence of m is independently an integer from 0 to 20. For example, in one embodiment, m is an integer of 11.

In various embodiments, each occurrence of n is independently an integer from 1 to 100. In some embodiments, each occurrence of n is independently an integer from 1 to 50. In some embodiments, each occurrence of n is independently an integer from 1 to 20. In some embodiments, each occurrence of n is independently an integer from 4 to 15. For example, in one embodiment, n is an integer of 4. In one embodiment, n is an integer of 5. In one embodiment, n is an integer of 6. In one embodiment, n is an integer of 7. In one embodiment, n is an integer of 8. In one embodiment, n is an integer of 9. In one embodiment, n is an integer of 10. In one embodiment, n is an integer of 11. In one embodiment, n is an integer of 12. In one embodiment, n is an integer of 13. In one embodiment, n is an integer of 14.

In various embodiments, each occurrence of o is independently an integer from 0 to 100. In some embodiments, each occurrence of o is independently an integer from 0 to 50. In some embodiments, each occurrence of o is independently an integer from 0 to 20. For example, in one embodiment, o is an integer of 0. In one embodiment, o is an integer of 1. In one embodiment, o is an integer of 2. In one embodiment, o is an integer of 3. In one embodiment, o is an integer of 4.

In various embodiments, each occurrence of p is independently an integer from 0 to 3. In some embodiments, each occurrence of p is independently an integer of 0 or 1. For example, in one embodiment, p is an integer of 0. In one embodiment, p is an integer of 1.

In various embodiments, each occurrence of r is independently an integer from 1 to 100. In some embodiments, each occurrence of r is independently an integer from 1 to 50. In some embodiments, each occurrence of r is independently an integer from 1 to 20. In some embodiments, each occurrence of r is independently an integer from 1 to 15. For example, in one embodiment, r is an integer of 1. In one embodiment, r is an integer of 2. In one embodiment, r is an integer of 3.

It is understood that, for a compound of Formula (I), (II), (III), (IV), or (V), R₁ and R₂ can each be, where applicable, selected from the groups described herein, and any group described herein for any of R₁ and R₂ can be combined, where applicable, with any group described herein for one or more of the remainder of R₁ and R₂.

In one embodiment, R₁ is alkyl. For example, in some embodiments, R₁ is C₁₋₁₁ alkyl. In one embodiment, R₁ is C₁ alkyl. In one embodiment, R₁ is C₂ alkyl. In one embodiment, R₁ is C₃ alkyl. In one embodiment, R₁ is C₄ alkyl. In one embodiment, R₁ is C₅ alkyl. In one embodiment, R₁ is C₆ alkyl. In one embodiment, R₁ is C₇ alkyl. In one embodiment, R₁ is C₈ alkyl. In one embodiment, R₁ is C₉ alkyl. In one embodiment, R₁ is C₁₀ alkyl. In one embodiment, R₁ is C₁₁ alkyl. In one embodiment, R₁ is methyl. In one embodiment, R₁ is ethyl. In one embodiment, R₁ is propyl. In one embodiment, R₁ is butyl. In one embodiment, R₁ is pentyl. In one embodiment, R₁ is hexyl. In one embodiment, R₁ is isopropyl. In one embodiment, R₁ is isobutyl. In one embodiment, R₁ is isopentyl. In one embodiment, R₁ is isohexyl. In one embodiment, R₁ is secbutyl. In one embodiment, R₁ is secpentyl. In one embodiment, R₁ is sechexyl. In one embodiment, R₁ is tertbutyl.

In one embodiment, R₁ is alkenyl. For example, in some embodiments, R₁ is C₂₋₁₀ alkenyl. In one embodiment, R₁ is C₂ alkenyl. In one embodiment, R₁ is C₃ alkenyl. In one embodiment, R₁ is C₄ alkenyl. In one embodiment, R₁ is C₅ alkenyl. In one embodiment, R₁ is C₆ alkenyl. In one embodiment, R₁ is C₇ alkenyl. In one embodiment, R₁ is C₈ alkenyl. In one embodiment, R₁ is C₉ alkenyl. In one embodiment, R₁ is C₁₀ alkenyl.

In one embodiment, R₁ is alkynyl. For example, in some embodiments, R₁ is C₂₋₁₀ alkynyl. In one embodiment, R₁ is C₂ alkynyl. In one embodiment, R₁ is C₃ alkynyl. In one embodiment, R₁ is C₄ alkynyl. In one embodiment, R₁ is C₈ alkynyl. In one embodiment, R₁ is C₆ alkynyl. In one embodiment, R₁ is C₇ alkynyl. In one embodiment, R₁ is C₈ alkynyl. In one embodiment, R₁ is C₉ alkynyl. In one embodiment, R₁ is C₁₀ alkynyl.

In one embodiment, R₁ is alkoxy. For example, in one embodiment, R₁ is —OR₃.

In one embodiment, R₁ is amino. For example, in one embodiment, R₁ is —N(R₃)(R₃).

In one embodiment, R₁ is aryl. For example, in one embodiment, R₁ is C₆-14 aryl.

In one embodiment, R₁ is heteroaryl. For example, in one embodiment, R₁ is a 5 to 14 membered heteroaryl ring having 1, 2, 3, 4, or 5 heteroatoms selected from the group consisting of nitrogen, oxygen, sulfur, and any combination thereof.

In one embodiment, R₂ is alkyl. For example, in some embodiments, R₂ is C₁₋₁₁ alkyl. In one embodiment, R₂ is C₁ alkyl. In one embodiment, R₂ is C₂ alkyl. In one embodiment, R₂ is C₃ alkyl. In one embodiment, R₂ is C₄ alkyl. In one embodiment, R₂ is C₅ alkyl. In one embodiment, R₂ is C₆ alkyl. In one embodiment, R₂ is C₇ alkyl. In one embodiment, R₂ is C₈ alkyl. In one embodiment, R₂ is C₉ alkyl. In one embodiment, R₂ is C₁₀ alkyl. In one embodiment, R₂ is C₁₁ alkyl. In one embodiment, R₂ is methyl. In one embodiment, R₂ is ethyl. In one embodiment, R₂ is propyl. In one embodiment, R₂ is butyl. In one embodiment, R₂ is pentyl. In one embodiment, R₂ is hexyl. In one embodiment, R₂ is isopropyl. In one embodiment, R₂ is isobutyl. In one embodiment, R₂ is isopentyl. In one embodiment, R₂ is isohexyl. In one embodiment, R₂ is secbutyl. In one embodiment, R₂ is secpentyl. In one embodiment, R₂ is sechexyl. In one embodiment, R₂ is tertbutyl.

In one embodiment, R₂ is alkenyl. For example, in some embodiments, R₂ is C₂₋₁₀ alkenyl. In one embodiment, R₂ is C₂ alkenyl. In one embodiment, R₂ is C₃ alkenyl. In one embodiment, R₂ is C₄ alkenyl. In one embodiment, R₂ is C₅ alkenyl. In one embodiment, R₂ is C₆ alkenyl. In one embodiment, R₂ is C₇ alkenyl. In one embodiment, R₂ is C₈ alkenyl. In one embodiment, R₂ is C₉ alkenyl. In one embodiment, R₂ is C₁₀ alkenyl.

In one embodiment, R₂ is alkynyl. For example, in some embodiments, R₂ is C₂₋₁₀ alkynyl. In one embodiment, R₂ is C₂ alkynyl. In one embodiment, R₂ is C₃ alkynyl. In one embodiment, R₂ is C₄ alkynyl. In one embodiment, R₂ is C₈ alkynyl. In one embodiment, R₂ is C₆ alkynyl. In one embodiment, R₂ is C₇ alkynyl. In one embodiment, R₂ is C₈ alkynyl. In one embodiment, R₁ is C₉ alkynyl. In one embodiment, R₂ is C₁₀ alkynyl.

In one embodiment, R₂ is alkoxy. For example, in one embodiment, R₂ is —OR₃.

In one embodiment, R₂ is amino. For example, in one embodiment, R₂ is —N(R₃)(R₃).

In one embodiment, R₂ is aryl. For example, in one embodiment, R₂ is C₆₋₁₄ aryl.

In one embodiment, R₂ is heteroaryl. For example, in one embodiment, R₂ is a 5 to 14 membered heteroaryl ring having 1, 2, 3, 4, or 5 heteroatoms selected from the group consisting of nitrogen, oxygen, sulfur, and any combination thereof.

In various embodiments, each occurrence of R₃ is independently selected from hydrogen, halogen, hydroxyl, hydroxylalkyl, alkoxy, amino, aminoalkyl, carboxyl, alkyl, cycloalkyl, heterocycloalkyl, alkenyl, cycloalkenyl, heterocycloalkenyl, alkynyl, cycloalkynyl, heterocycloalkynyl, aryl, arylalkyl, heteroaryl, heteroarylalkyl, arylalkenyl, heteroarylalkenyl, arylalkynyl, heteroarylalkynyl, carbamate, guanidine, or guanidine alkyl.

For example, in some embodiments, the arylalkyl is a C₁₋₆ alkyl-C₆₋₁₄ aryl. In some embodiments, the arylalkenyl is a C₂₋₆ alkenylene-C₆₋₁₄ aryl. In some embodiments, the arylalkynyl is a C₂₋₆ alkynylene-C₆₋₁₄ aryl. In some embodiments, the heteroarylalkyl is a C₁₋₆ alkyl-5 to 14 membered heteroaryl ring having 1, 2, 3, 4, or 5 heteroatoms selected from the group consisting of nitrogen, oxygen, sulfur, and any combination thereof. In some embodiments, the heteroarylalkenyl is a C₂₋₆ alkenyl-5 to 14 membered heteroaryl ring having 1, 2, 3, 4, or 5 heteroatoms selected from the group consisting of nitrogen, oxygen, sulfur, and any combination thereof. In some embodiments, the heteroarylalkynyl is a C₂₋₆ alkynyl-5 to 14 membered heteroaryl ring having 1, 2, 3, 4, or 5 heteroatoms selected from the group consisting of nitrogen, oxygen, sulfur, and any combination thereof.

In some embodiments, the cycloalkyl is a C₃₋₁₀ cycloalkyl. In some embodiments, the cycloalkenyl is a C₃₋₁₀ cycloalkenyl. In some embodiments, the cycloalkynyl is a C₃-10 cycloalkynyl. In some embodiments, the heterocyclyl is a 3-10 membered heterocyclyl having 1, 2, or 3 heteroatoms selected from the group consisting of nitrogen, oxygen, sulfur, and any combination thereof.

For example, in some embodiments, each occurrence of R₁, R₂, or R₃ is independently selected from

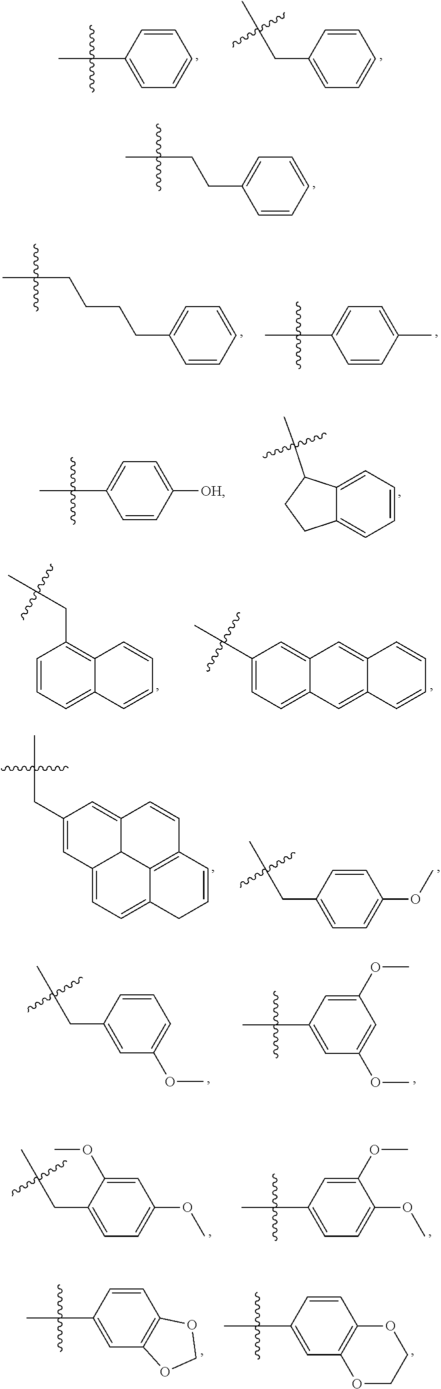

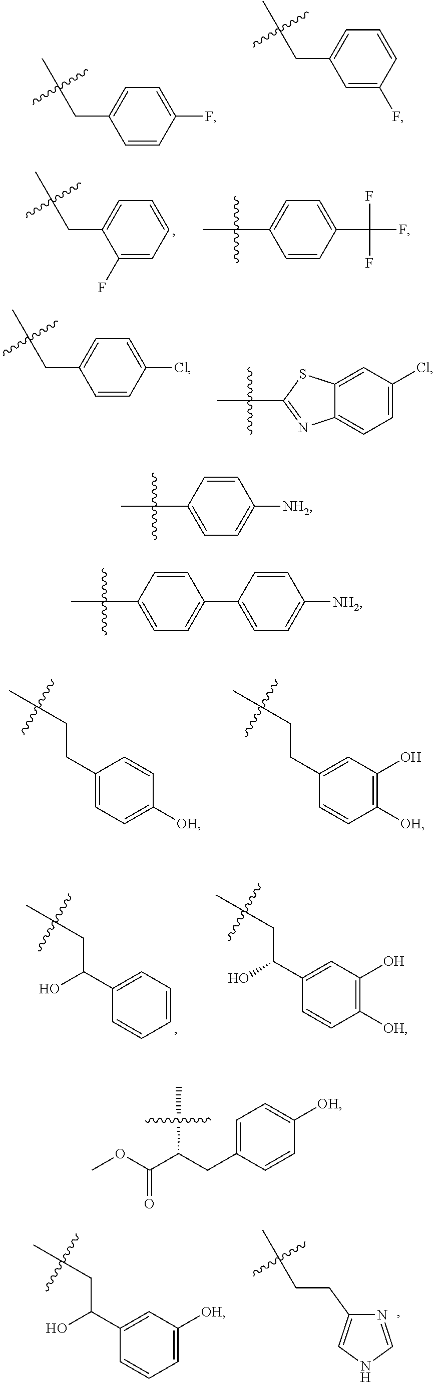

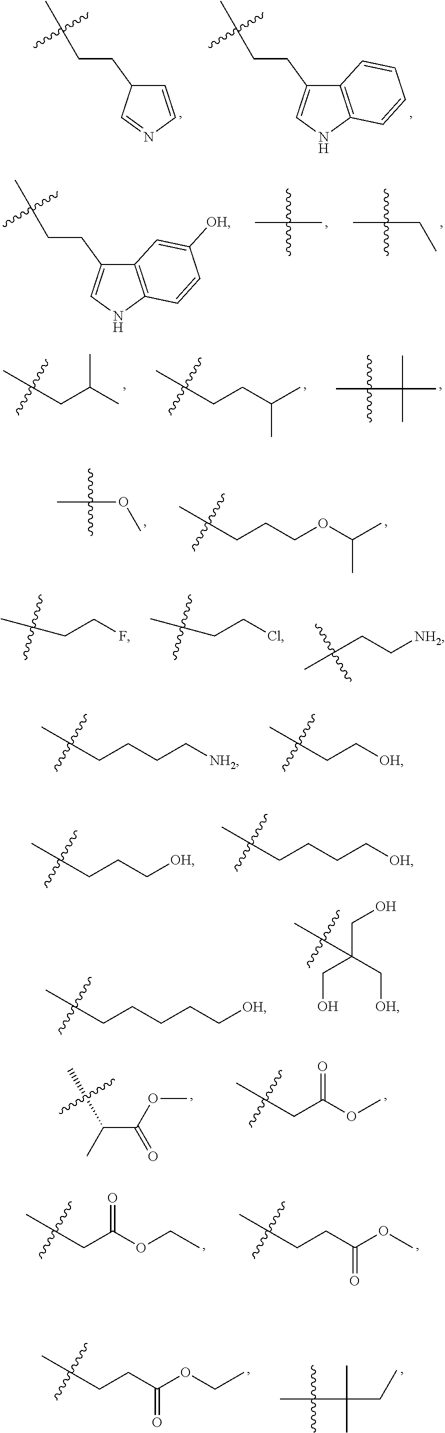

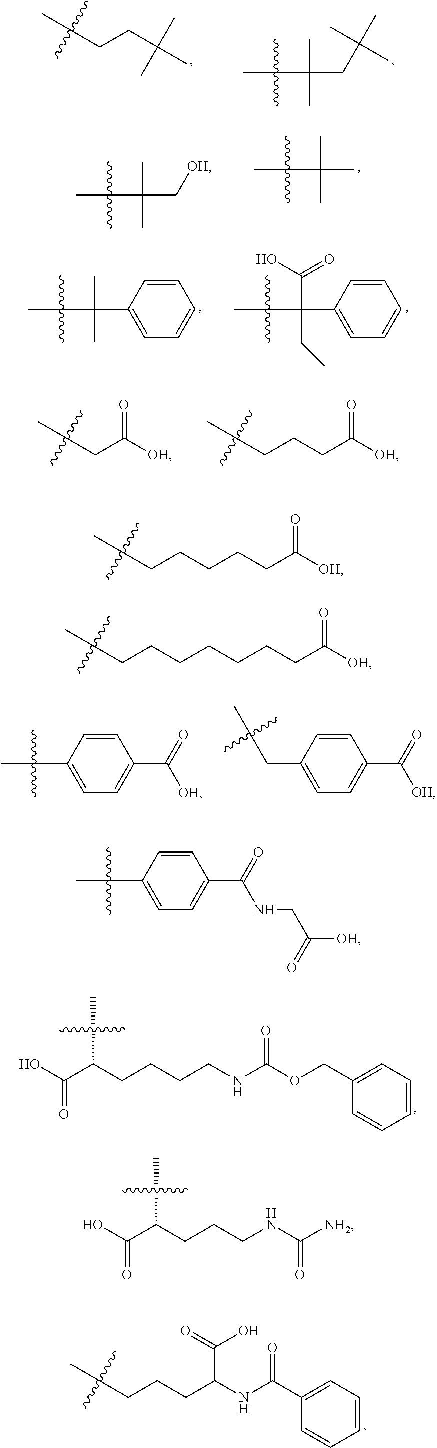

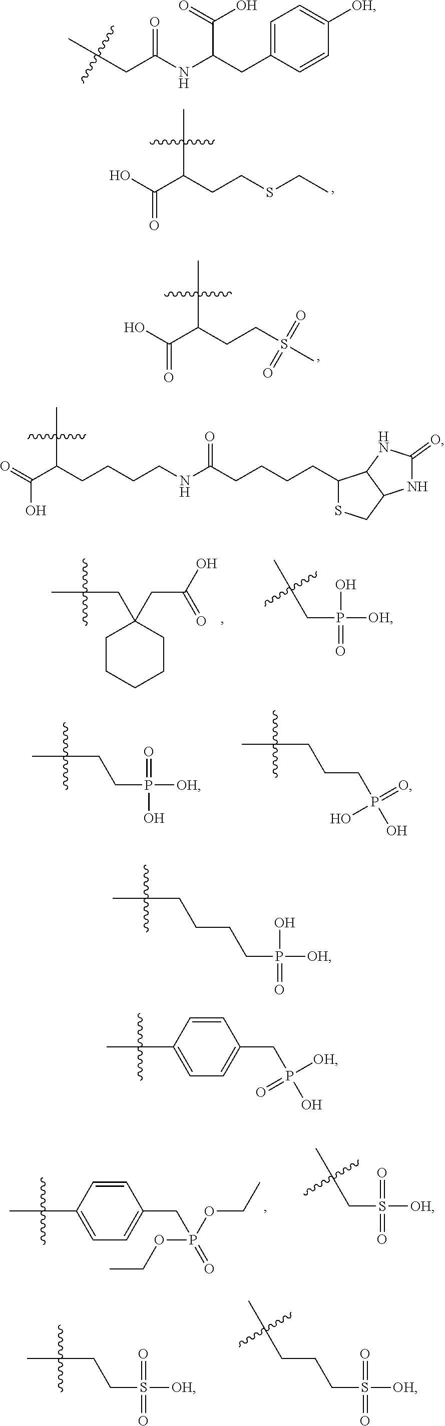

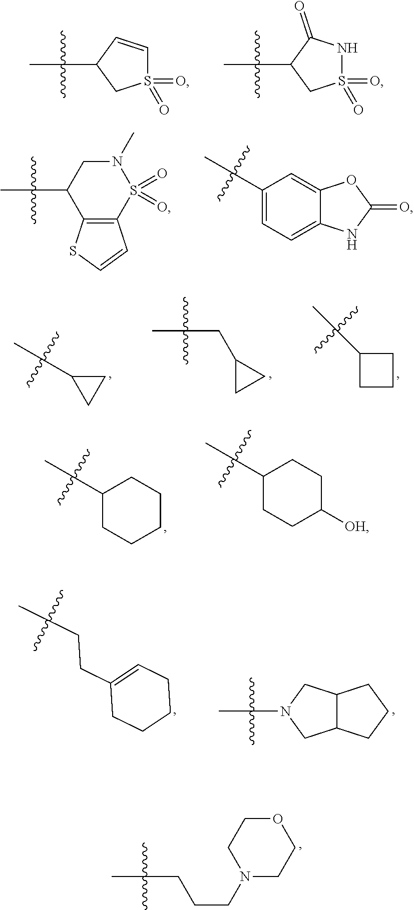

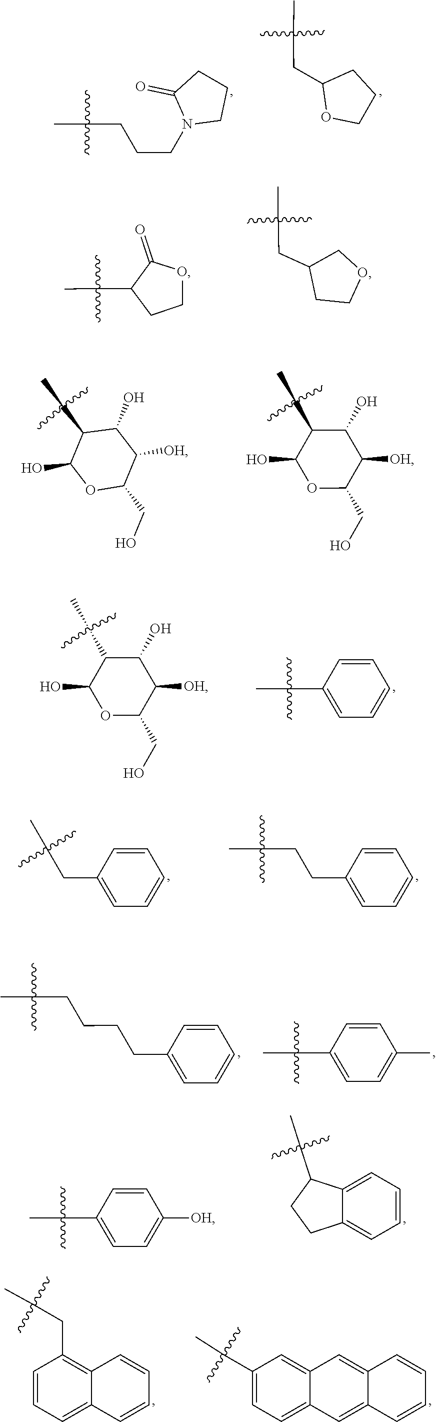

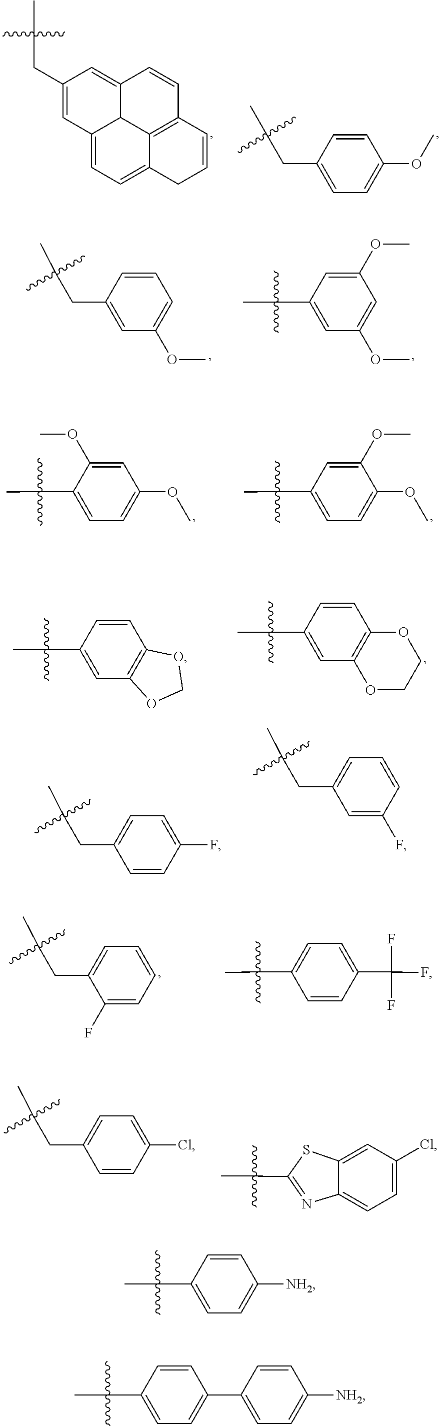

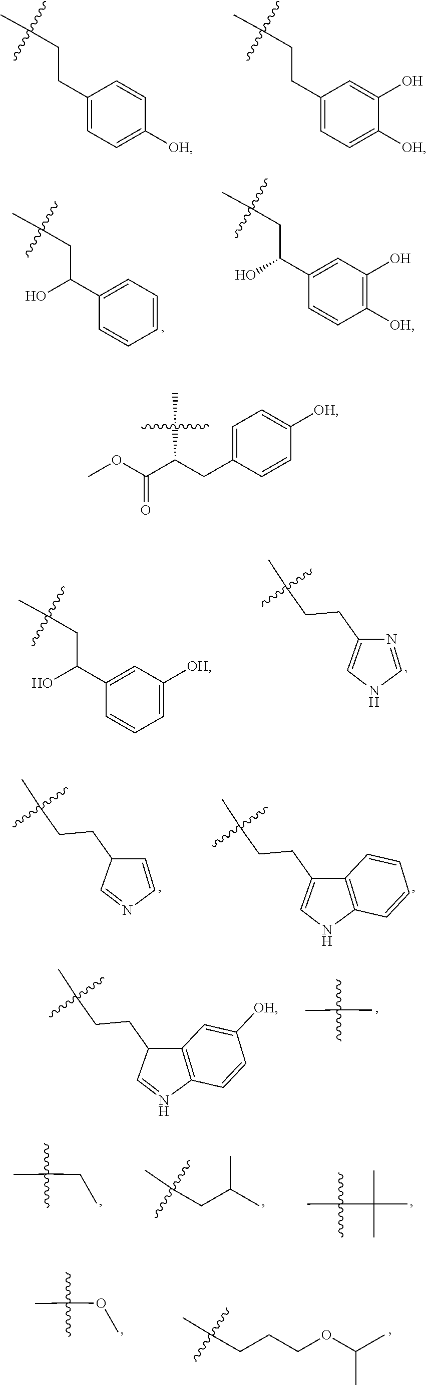

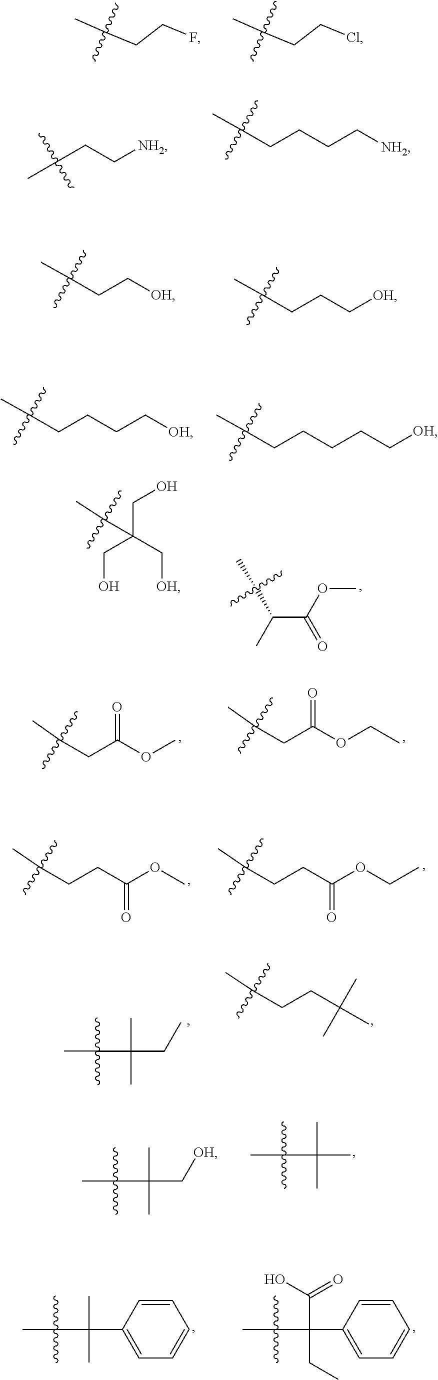

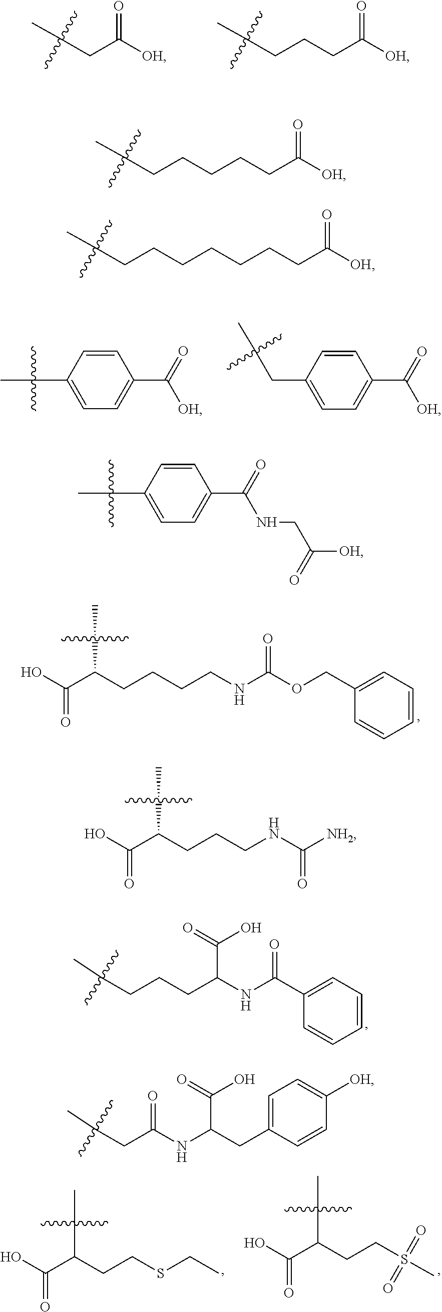

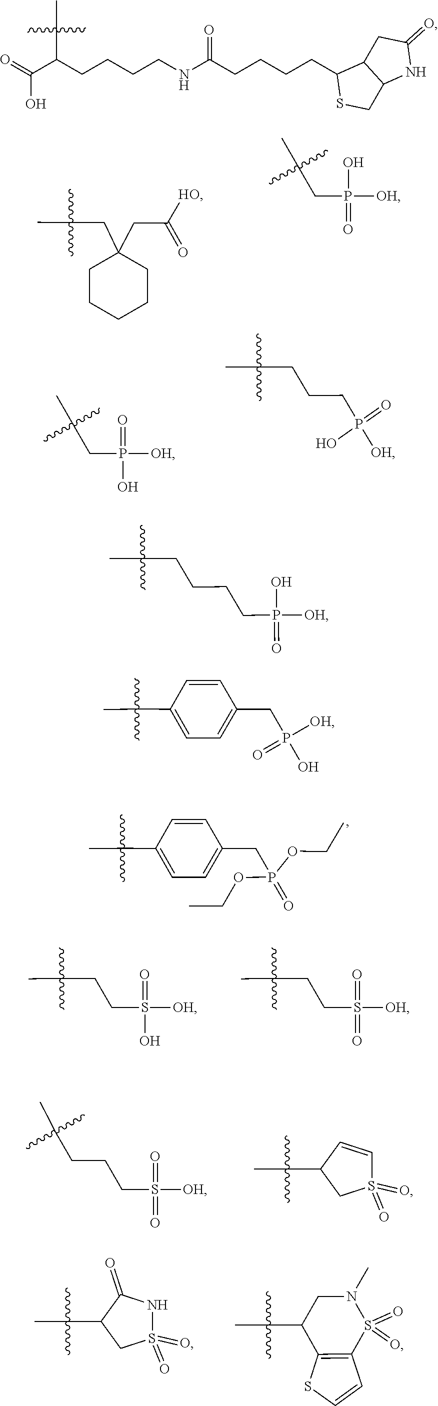

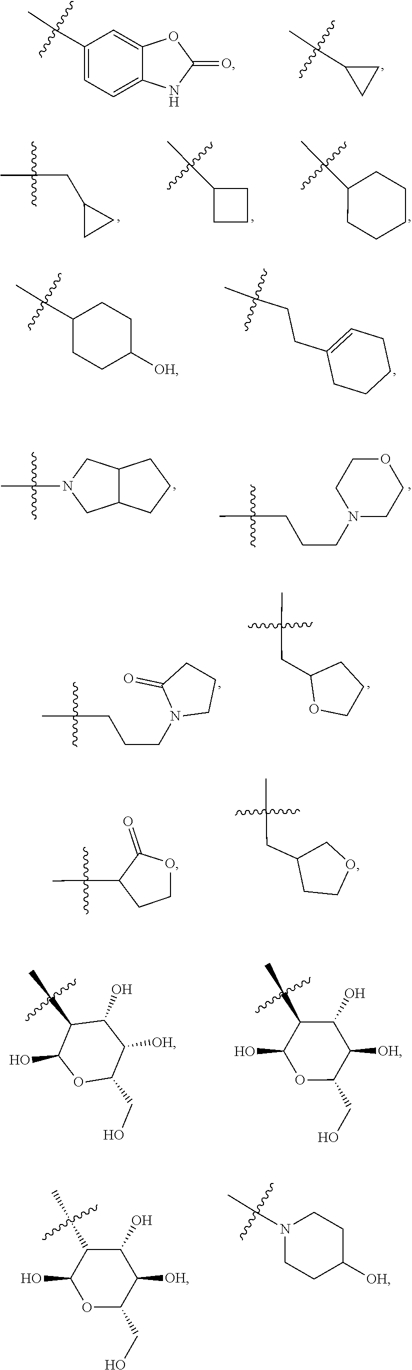

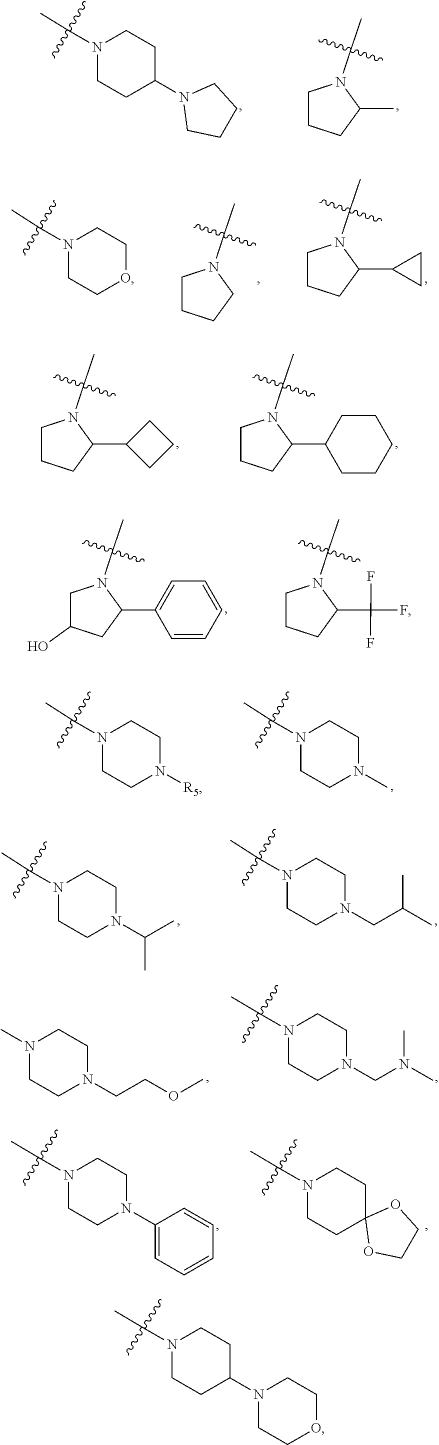

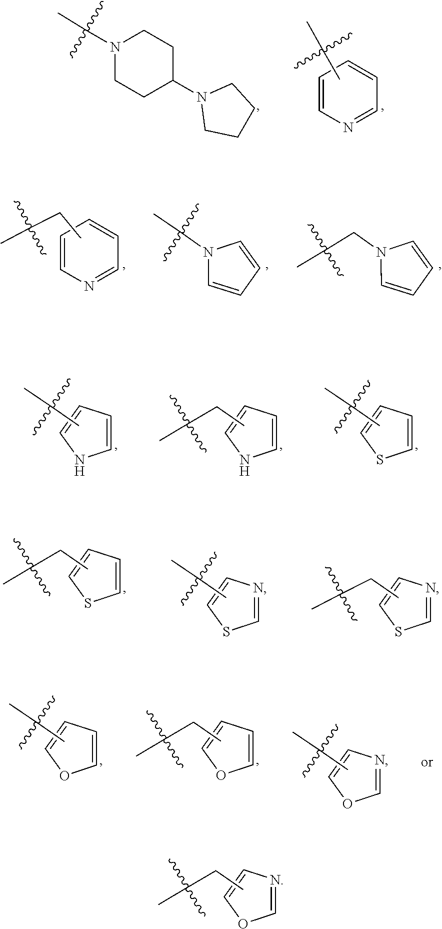

Thus, for example, in some embodiments, the compound having the structure of Formula (I) is a compound having the structure of

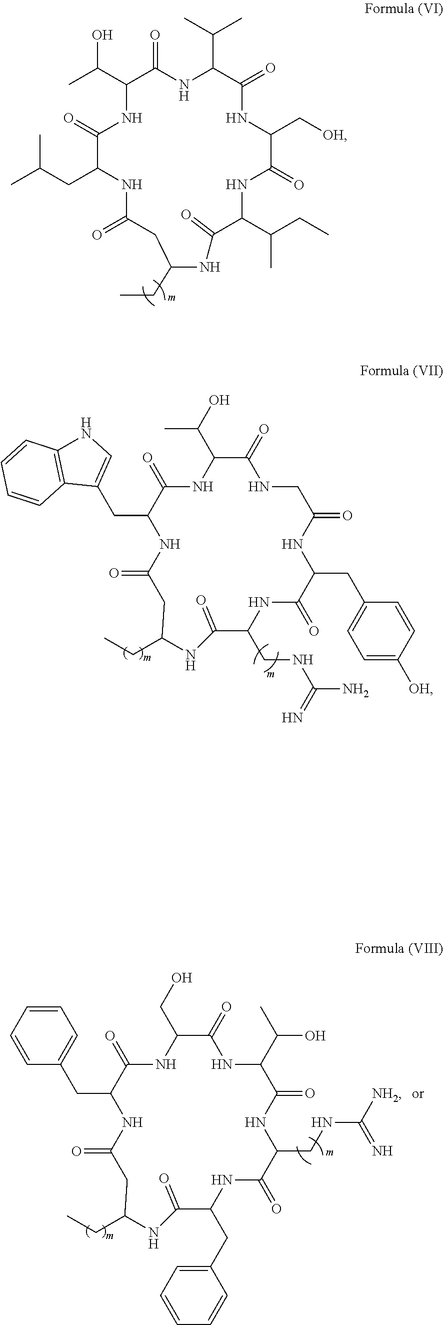

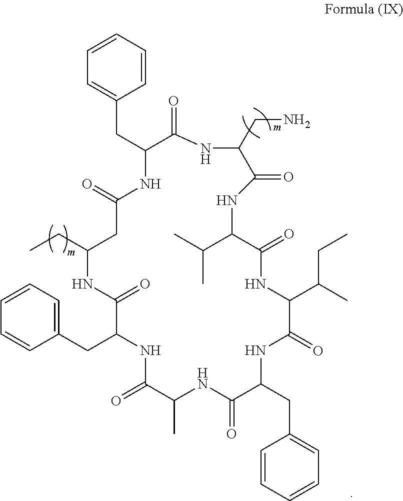

In some embodiments, the compound having the structure of Formula (II) is a compound having the structure of Formula (X)

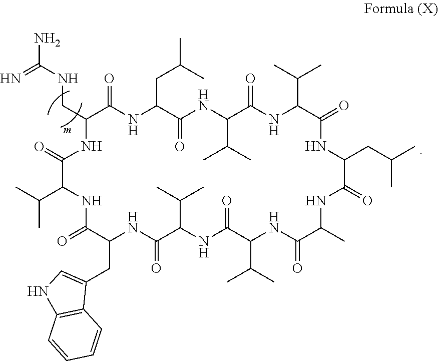

In some embodiments, the compound having the structure of Formula (III) is a compound having the structure of

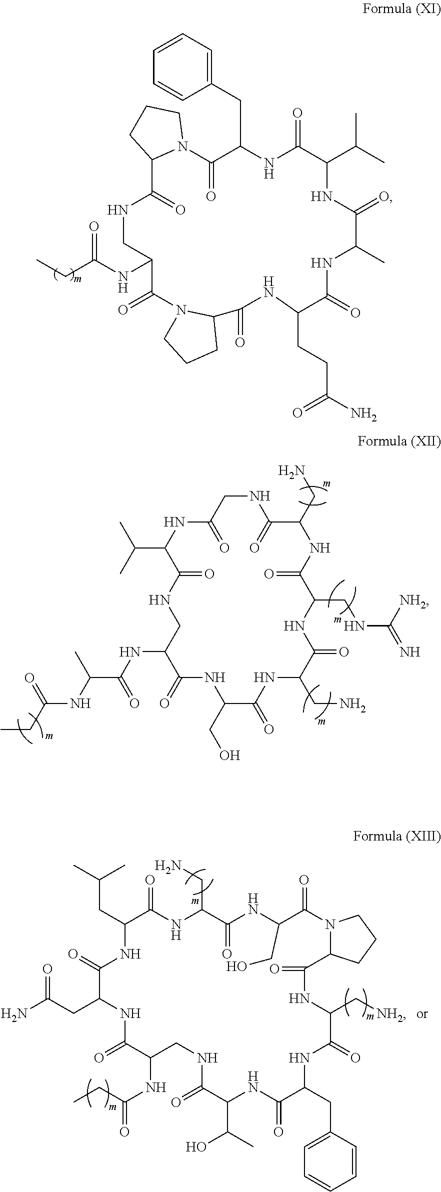

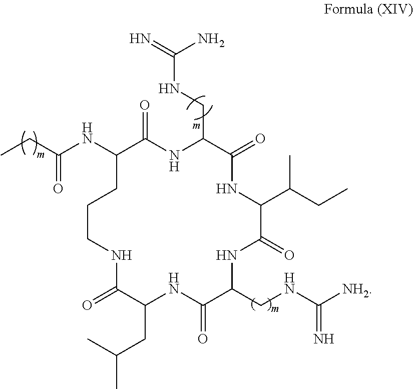

In some embodiments, each occurrence of m is independently an integer from 1 to 20. For example, in one embodiment, each occurrence of m is independently an integer 3 or 11.

In various embodiments, the compound having the structure of Formula (I), (II), or (III) is a nonribosomal peptide. In some embodiments, the compound having the structure of Formula (I), (II), or (III) is a synthetic-bioinformatic natural product (syn-BNP). In some embodiments, the compound having the structure of Formula (I), (II), or (III) is a syn-BNP cyclic peptide antibiotic (syCPA).

In various aspects of the invention, the compound having the structure of Formula (I), (II), or (III) modulates the growth or proliferation of at least one microorganism, cell wall biosynthesis, cell lysis, membrane depolarization mitochondrial ClpP protease, or any combination thereof. In some embodiments, the compound reduces or inhibits the growth or proliferation of at least one microorganism, reduces cell wall biosynthesis, induces cell lysis, induces membrane depolarization, dysregulates at least one mitochondrial ClpP protease, or any combination thereof.

In some embodiments, the compounds of the present invention have an MIC value for any of the microorganism disclosed herein of less than about 10,000 μg/mL, about 5000 μg/mL, about 1000 μg/mL, about 900 μg/mL, about 800 μg/mL, about 700 μg/mL, about 600 μg/mL, about 500 μg/mL, about 400 μg/mL, about 300 μg/mL, about 200 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, about 40 μg/mL, about 35 μg/mL, about 30 μg/mL, about 25 μg/mL, about 20 μg/mL, about 15 μg/mL, about 10 μg/mL, about 9 μg/mL, about 8 μg/mL, about 7 μg/mL, about 6 μg/mL, about 5 μg/mL, about 4 μg/mL, about 3 μg/mL, about 2 μg/mL, about 1 μg/mL, about 0.5 μg/mL, about 0.1 μg/mL, about 0.05 μg/mL, about 0.01 μg/mL, about 0.005 μg/mL, about 0.001 μg/mL, about 0.0005 μg/mL, about 0.0001 μg/mL, about 0.05 ng/mL, about 0.01 ng/mL, about 0.005 ng/mL, or about 0.001 ng/mL, or lower, including all values and ranges therebetween.

In various aspects of the invention, the compound having the structure of Formula (I), (II), or (III) modulates the growth or proliferation of at least one microorganism. In some embodiments, the compound reduces the growth or proliferation of at least one microorganism. In some embodiments, the compound inhibits the growth or proliferation of at least one microorganism.

In some embodiments, the compound results in reduced growth or proliferation of at least one microorganism that is reduced or decreased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the compound results in the growth or proliferation of at least one microorganism that is at least about 0.01 fold less than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or more, including all values and ranges in-between.

In some embodiments, the compound reduces or inhibits the growth or proliferation of at least one microorganism at a minimal inhibitory concentration (MIC) between around 0.001 μg/mL and 10,000 μg/mL. For example, in one embodiment, the compound reduces or inhibits the growth or proliferation of at least one microorganism at a MIC around 8 μg/mL.

In various aspects of the invention, the compound having the structure of Formula (I), (II), or (III) modulates the cell wall biosynthesis of at least one microorganism. In some embodiments, the compound reduces the cell wall biosynthesis of at least one microorganism. In some embodiments, the compound inhibits the cell wall biosynthesis of at least one microorganism.

In some embodiments, the compound results in the cell wall biosynthesis of at least one microorganism that is reduced or decreased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the compound results in reduced cell wall biosynthesis of at least one microorganism that is at least about 0.01 fold less than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or more, including all values and ranges in-between.

In some embodiments, the compound reduces or inhibits the cell wall biosynthesis of at least one microorganism at a MIC between around 0.001 μg/mL and 10,000 μg/mL. For example, in one embodiment, the compound reduces or inhibits the cell wall biosynthesis of at least one microorganism at a MIC around 8 μg/mL.

In various aspects of the invention, the compound having the structure of Formula (I), (II), or (III) modulates cell lysis of at least one microorganism. In some embodiments, the compound induces cell lysis of at least one microorganism.

In some embodiments, the compound results in induced cell lysis of at least one microorganism that is increased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the compound results in induced cell lysis of at least one microorganism that is at least about 0.01 fold higher than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or more, including all values and ranges in-between.

In some embodiments, the compound induces cell lysis of at least one microorganism at a MIC between around 0.001 μg/mL and 10,000 μg/mL. For example, in one embodiment, the compound induces cell lysis of at least one microorganism at a MIC around 8 μg/mL.

In various aspects of the invention, the compound having the structure of Formula (I), (II), or (III) modulates membrane depolarization of at least one microorganism. In some embodiments, the compound induces membrane depolarization of at least one microorganism.

In some embodiments, the compound results in induced membrane depolarization of at least one microorganism that is increased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the compound results in induced membrane depolarization of at least one microorganism that is at least about 0.01 fold higher than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or more, including all values and ranges in-between.

In some embodiments, the compound induces membrane depolarization of at least one microorganism at a MIC between around 0.001 μg/mL and 10,000 μg/mL. For example, in one embodiment, the compound induces membrane depolarization of at least one microorganism at a MIC around 8 μg/mL.

In various aspects of the invention, the compound having the structure of Formula (I), (II), or (III) modulates at least one mitochondrial ClpP protease of at least one microorganism. In some embodiments, the compound dysregulates at least one mitochondrial ClpP protease of at least one microorganism.

In some embodiments, the compound results in a dysregulation of at least one mitochondrial ClpP protease of at least one microorganism that is increased by at least about 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some embodiments, the compound results in a dysregulation of at least one mitochondrial ClpP protease of at least one microorganism that is at least about 0.01 fold higher than the comparator (e.g., control), e.g., about 0.01 fold, about 0.05 fold, about 0.10 fold, about 0.25 fold, about 0.50 fold, about 0.75 fold, about 1.0 fold, about 1.25 fold, 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 11 fold, about 12 fold, about 13 fold, about 14 fold, about 15 fold, about 16 fold, about 17 fold, about 18 fold, about 19 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or more, including all values and ranges in-between.

In some embodiments, the compound dysregulates at least one mitochondrial ClpP protease of at least one microorganism at a MIC between around 0.001 μg/mL and 10,000 μg/mL. For example, in one embodiment, the compound dysregulates at least one mitochondrial ClpP protease of at least one microorganism at a MIC around 8 μg/mL.

In various embodiments, the compound having the structure of Formula (I), (II), or (III) is an antimicrobial compound. In some embodiments, the microorganism is a bacterium, virus, fungus, parasite, and any combination thereof. In one embodiment, the microorganism is resistant to at least one antibiotic.

Examples of microorganisms include, but are not limited to bacteria, such as Acinetobacter baumannii, Actinomyces israelii, Bacillus anthracis, Bacillus cereus, Bacillus subtilis, Bacteroides fragilis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtherias, Ehrlichia canis, Ehrlichia chaffeensis, Enterobacter cloacae, Enterococcus faecalis, Enterococcus faecium, Escherichia coli, Francisella tularensis, Haemophilus influenzae, Helicobacter pylori, Klebsiella pneumoniae, Lactobacillus rhamnosus, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Proteus mirabills, Pseudomonas aeruginosa, Nocardia asteroids, Rickettsia rickettsia, Salmonella enterica, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Candida species, such as Candida albicans, Candida tropicalis, Candida glabrata, Candida parapsilosis, Candida krusei, Candida lusitaniae, Candida kefyr, Candida guilliermondii, and Candida dubliniensis, Yersinia pestis, Yersinia enterocolitica, Yersinia pseudotuberculosis, and any combination thereof.

In various embodiments, the compounds of the present invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or any combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase.

In one embodiment, the compounds of the disclosure may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

In various embodiments, the present invention also relates, in part, to compositions comprising at least one compound of the present invention. In one embodiment, the invention provides a therapeutic composition comprising at least one compound having the structure of Formula (I), (II), (III), or any combination thereof. In one embodiment, a mixture of one or more isomer is utilized as the therapeutic compound described herein.

### Methods of Preparation and Screening of Antimicrobial Compounds

In some aspects, the present invention also provides methods of preparing, screening, and/or identifying the compounds of the present invention. In one embodiment, the present invention provides a method of preparing, screening, and/or identifying the antimicrobial compounds of the present invention. In one embodiment, the present invention provides a method of preparing, screening, and/or identifying the syn-BNP of the present invention. In one embodiment, the present invention provides a method of preparing, screening, and/or identifying the syCPA of the present invention.

In some embodiments, the method comprises: a) analyzing nonribosomal peptide synthase (NRPS) gene clusters, wherein the NRPS gene clusters encode one or more peptides; b) identifying one or more peptides that are encoded by the NRPS gene clusters, wherein the peptides comprise at least 4 amino acids; c) synthesizing the peptides; and d) covalently cyclizing the peptides to generate at least one syn-BNP.

The compounds described herein, and other related compounds having different substituents are synthesized using techniques and materials described. Compounds described herein are synthesized using any suitable procedures starting from compounds that are available from commercial sources, or are prepared using procedures described herein.

In some embodiments, the method further comprises e) exposing the syn-BNP to at least one microorganism; and f) identifying the syn-BNP that reduces the level of at least one microorganism. For example, in one embodiment, the present invention provides a method of preparing at least one syCPA, the method comprising of: a) analyzing nonribosomal peptide synthase (NRPS) gene clusters, wherein the NRPS gene clusters encode one or more peptides; b) identifying one or more peptides that are encoded by the NRPS gene clusters, wherein the peptides comprise at least 4 amino acids; c) synthesizing the peptides; d) covalently cyclizing the peptides to generate at least one syn-BNP; e) exposing the syn-BNP to at least one bacterium; and f) identifying the syn-BNP that reduces the level of at least one bacterium.

In various embodiments of the methods of the invention, the syn-BNP is an antimicrobial compound when the level (e.g., activity, expression, concentration, level, etc.) of at least one microorganism is determined to be decreased or reduced when compared to a comparator. For example, in one embodiment, the syn-BNP is an antimicrobial compound when the level (e.g., activity, expression, concentration, level, etc.) of at least one bacterium is determined to be decreased or reduced when compared to a comparator. In one embodiment, the syn-BNP is a syCPA when the level (e.g., activity, expression, concentration, level, etc.) of at least one bacterium is determined to be decreased or reduced when compared to a comparator.

In various embodiments of the methods of the invention, the level (e.g., activity, expression, concentration, level, etc.) of microorganism (e.g., bacterium, virus, fungus, parasite, etc.) is determined to be decreased or reduced when the level (e.g., activity, expression, concentration, level, etc.) of microorganism (e.g., bacterium, virus, fungus, parasite, etc.) is decreased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of microorganism (e.g., bacterium, virus, fungus, parasite, etc.) is determined to be decreased or reduced when the level (e.g., activity, expression, concentration, level, etc.) of microorganism (e.g., bacterium, virus, fungus, parasite, etc.) is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the syn-BNP is an antimicrobial compound when the level (e.g., activity, expression, concentration, level, etc.) of microorganism (e.g., bacterium, virus, fungus, parasite, etc.) is decreased or reduced in the biological sample as compared to a comparator. For example, in one embodiment, the syn-BNP is an antimicrobial compound when the level (e.g., activity, expression, concentration, level, etc.) of microorganism (e.g., bacterium, virus, fungus, parasite, etc.) is decreased by at least 1 fold, at least 1.1 fold, at least 1.2 fold, at least 1.3 fold, at least 1.4 fold, or at least 1.5 fold.

In one embodiment, the method comprises using a multi-dimensional non-linear algorithm to determine if the level (e.g., activity, expression, concentration, level, etc.) of at least one microorganism is statistically different than the level in a comparator sample. In some embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

In one embodiment, the method comprises detecting one or more microorganism in a biological sample of the subject. In some embodiments, the level of one or more microorganism in the biological test sample of the subject is compared with the level of the microorganism in a comparator. Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In one embodiment, the comparator is a level of the at least one microorganism in a sample obtained from a subject not having a disease or disorder, such as bacterial infection. In one embodiment, the comparator is a level of the one or more biomarker in a sample obtained from a subject known not to have a disease or disorder, such as bacterial infection.

In certain embodiments, the biological sample obtained from the subject comprises gastrointestinal tissue of the subject, including gastrointestinal tissue excised during biopsy. Biological samples may be of any biological tissue or fluid. Frequently the sample will be a “clinical sample” which is a sample derived from a patient. The biological sample may contain any biological material suitable for detecting the desired microorganisms, and may comprise cellular and/or non-cellular material obtained from the individual. A biological sample can be obtained by appropriate methods, such as, by way of examples, blood draw, fluid draw, biopsy, or surgical resection. Examples of such samples include but are not limited to blood, lymph, urine, gastrointestinal fluid, semen, and biopsies. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, tissue sample obtained during surgical resection, and archival samples with known diagnosis, treatment and/or outcome history. In certain embodiments, the biological sample comprises gastrointestinal tissue. In certain embodiments, the biological sample comprises gastrointestinal tissue of a subject having gastrointestinal cancer.

In some embodiments, the methods of the invention use live cells to perform experiments as the basis for the identification of an antimicrobial compounds.

When microorganisms are detected to alter particular parameters, identification of the compounds responsible for the altered parameter is performed. Various methods are known in the art for identifying an unknown compound in a complex mixture. Individual components may be separated, analyzed, and characterized using methods known to those skilled in the art. In a non-limiting embodiment, the individual components may be partially or completely purified using, for example, chromatographic methods (such as, but not limited to, high performance liquid chromatography (HPLC), silica gel chromatography or alumina chromatography), selective crystallization or precipitation, or selective solvent extraction. In another non-limiting embodiment, the partially or completely purified components of the library may be analyzed or characterized using methods such as, but not limited to, nuclear magnetic resonance (NMR), mass spectrometry (MS), liquid chromatography-mass spectrometry (LC-MS), ultraviolet-visible (UV-vis) spectroscopy, and infrared (IR) spectroscopy. The information derived from these methods may be used to establish the structure of the specific components of the library.

In one embodiment, the methods of the invention relate to high throughput screening methods and automated screening of large quantities of antimicrobial test compounds to identify specific microorganism that interact with the antimicrobial compounds. In some embodiments, the assays and methods comprise high content screening (HCS) of suitable antimicrobial compounds. Typically, HCS is an automated system to enhance the throughput of the screening process. However, the present invention is not limited to the speed or automation of the screening process.

In one embodiment, the assay of the invention may also be used to test delivery vehicles. These may be of any form, from conventional pharmaceutical formulations, to gene delivery vehicles. For example, the assay may be used to compare the effects of the same compound administered by two or more different delivery systems (e.g. a depot formulation and a controlled release formulation). Thus, the antimicrobial test compound may be delivered by a delivery vehicle of any appropriate type with or without any associated therapeutic agent.

In one embodiment, compounds are evaluated alone. In another embodiment, compounds are evaluated when delivered along with a delivery vehicle. Non-limiting examples of delivery vehicles include polymersomes, vesicles, micelles, plasmid vectors, viral vectors, and the like. As described elsewhere herein, the antimicrobial test compounds are evaluated for their ability to interact with at least one microorganism. In one embodiment, the methods of the invention comprise selecting an antimicrobial test compound that inhibits or reduces at least one microorganism. In one embodiment, the antimicrobial test compound are delivered along with other known agents to determine whether the antimicrobial test compounds exhibit interference or synergy with other agents.

The antimicrobial test compound may be added to the assay method to be tested by any suitable means. For example, the antimicrobial test compound may be injected into the cells of the assay, or it can be added to the nutrient medium and allowed to diffuse into the cells.

In one embodiment, the screening methods involve providing a library containing a large number of antimicrobial test compounds, at least one of which potentially having an activity through its interaction with at least one microorganism. Such a library is then screened in one or more assays, as described herein, to identify those library members (particular chemical species or subclasses) that display a desired characteristic activity. The compounds thus identified can serve as conventional “hit compounds” or can themselves be used as potential or actual therapeutics. It is typical to that new chemical entities with useful properties are generated by identifying a chemical compound (called a “hit compound”) with some desirable property or activity, and evaluating the property of those compounds. The invention includes such hit compounds, as well as compounds derived from such hit compounds.

Thus, the present invention also relates to methods of screening and identifying drug test compounds to identify drug compounds that reduce or inhibit at least one microorganism. In one embodiment, the invention comprises assessing whether the drug test compound reduces or inhibits at least one microorganism. In some embodiments, the microorganism is a known microorganism. In one embodiments, the microorganism is a bacterium. In other embodiments, the microorganism is an unknown microorganism.

In various embodiments, the methods of screening and identifying antimicrobial compounds to identify drug compounds that reduce or inhibit at least one microorganism are any of the methods described herein.

In various aspects, the present invention also relates, in part, to methods of preparing compositions comprising at least one compound of the present invention. In one embodiment, the invention provides methods of preparing a therapeutic composition comprising at least one compound having the structure of Formula (I), (II), (III), or any combination thereof. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

In one aspect of the invention, the therapeutic composition is used to prevent or treat a disease or disorder in a subject in need thereof. In various embodiments, the disease or disorder is associated with at least one microorganism (e.g., bacterium).

### Methods of Treating or Preventing Diseases or Disorders

The present invention relates, in part, to a method of modulating the growth or proliferation of at least one microorganism, cell wall biosynthesis of at least one microorganism, cell lysis of at least one microorganism, membrane depolarization of at least one microorganism, dysregulation of at least one mitochondrial ClpP protease, or any combination thereof in a subject in need thereof. For example, in one embodiment, the present invention provides a method of preventing, reducing, or inhibiting the growth or proliferation of at least one microorganism in a subject in need thereof. In another embodiment, the present invention provides a method of preventing, reducing, or inhibiting cell wall biosynthesis of at least one microorganism in a subject in need thereof. In another embodiment, the present invention provides a method of inducing membrane depolarization of at least one microorganism in a subject in need thereof. In another embodiment, the present invention provides a method of dysregulating at least one mitochondrial ClpP protease.

For example, in one aspect of the invention, the method of dysregulating at least one mitochondrial ClpP protease leads to apoptotic cell death. Thus, in various aspects, the present invention provides methods of preventing or treating cancer in a subject in need thereof by administering a therapeutically effective amount of at least one compound or a composition thereof described herein to the subject. In various embodiments, the compound or a composition thereof dysregulates at least one mitochondrial ClpP protease. In some embodiments, the compound or a composition thereof dysregulates at least one mitochondrial ClpP protease leading to apoptotic cell death. In some embodiments, the compound or a composition thereof dysregulates at least one mitochondrial ClpP protease leading to apoptotic cancer cell death.

The present invention also relates, in part, to a method of treating or preventing a disease or disorder in a subject in need thereof. In various embodiments, the methods of invention comprise administering a therapeutically effective amount of at least one compound or a composition thereof described herein to the subject.

In various embodiments, the disease or disorder is associated with at least one microorganism. In various embodiments, the disease or disorder is an infection, cancer, or any combination thereof. In some embodiments, the infection is a bacterial infection, viral infection, fungal infection, parasitic infection, or any combination thereof.

In various embodiments, the infection is caused by any microorganism described herein. For example, in some embodiments, the bacterial infection is caused by a bacterium selected from the group consisting of Acinetobacter baumannii, Actinomyces israelii, Bacillus anthracis, Bacillus cereus, Bacillus subtilis, Bacteroides fragilis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtherias, Ehrlichia canis, Ehrlichia chaffeensis, Enterobacter cloacae, Enterococcus faecalis, Enterococcus faecium, Escherichia coli, Francisella tularensis, Haemophilus influenzae, Helicobacter pylori, Klebsiella pneumoniae, Lactobacillus rhamnosus, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Proteus mirabills, Pseudomonas aeruginosa, Nocardia asteroids, Rickettsia rickettsia, Salmonella enterica, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Candida species, such as Candida albicans, Candida tropicalis, Candida glabrata, Candida parapsilosis, Candida krusei, Candida lusitaniae, Candida kefyr, Candida guilliermondii, and Candida dubliniensis, Yersinia pestis, Yersinia enterocolitica, Yersinia pseudotuberculosis, or any combination thereof.

The following are non-limiting examples of cancers that can be treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

In some embodiments, the disease or disorder is associated with the level (e.g., activity, expression, level, etc.) of at least one microorganism. In one embodiment, the disease or disorder is associated with an increased level (e.g., activity, expression, concentration, level, etc.) of at least one microorganism.

In one embodiment, the disease or disorder is associated with an increased level of at least one microorganism when the level (e.g., activity, expression, concentration, level, etc.) of at least one microorganism is increased when compared to a comparator. In various embodiments of the methods of the invention, the level (e.g., activity, expression, concentration, level, etc.) of at least one microorganism is determined to be increased when the relevant microorganism is differentially expressed as compared to a comparator. In certain embodiments, the comparator may be at the level of the relevant microorganism in a subject not having a disease or disorder associated with increased level (e.g., activity, expression, concentration, level, etc.) of at least one microorganism, a subject not at risk of developing a disease or disorder associated with increased level (e.g., activity, expression, concentration, level, etc.) of at least one microorganism, a population not having a disease or disorder associated with increased level (e.g., activity, expression, concentration, level, etc.) of at least one microorganism, or a population not having a risk of developing a disease or disorder associated with increased level (e.g., activity, expression, concentration, level, etc.) of at least one microorganism.

In various embodiments, the method comprises modulating the level (e.g., activity, expression, level, etc.) of the at least one microorganism thereof in the subject. In some embodiments, the method comprises modulating the level of the at least one microorganism in the subject. In one embodiment, the method comprises reducing the level (e.g., activity, expression, level, etc.) of the at least one microorganism in the subject.

In some embodiments, the methods of the present invention modulate the level (e.g., activity, expression, level, etc.) of at least one microorganism. For example, in some embodiments, the methods of the present invention modulate the level (e.g., activity, expression, level, etc.) of at least one bacterium described herein. In some embodiments, the methods of the present invention reduce the level (e.g., activity, expression, level, etc.) of at least one microorganism (e.g., bacterium, virus, parasite, etc.). For example, in one embodiment, the level (e.g., activity, expression, level, etc.) of at least one microorganism is determined to be a decreased or reduced level of microorganism when the level (e.g., activity, expression, concentration, level, etc.) of at least one microorganism is decreased by at least 1 fold, at least 1.1 fold, at least 1.2 fold, at least 1.3 fold, at least 1.4 fold, or at least 1.5 fold. For example, in one embodiment, the level of at least one bacterium is determined to be a decreased or reduced level of bacterium when the level (e.g., activity, expression, concentration, level, etc.) of at least one bacterium is decreased by at least 1 fold, at least 1.1 fold, at least 1.2 fold, at least 1.3 fold, at least 1.4 fold, or at least 1.5 fold.

In some aspects of the invention, the methods of the present invention modulate the level (e.g., activity, expression, level, etc.) of at least one microorganism by administering at least one antimicrobial compound of the present invention to a subject in need thereof. In some embodiments, the methods of the present invention reduce or eliminate the level (e.g., activity, expression, level, etc.) of at least one microorganism by administering at least one antimicrobial compound of the present invention to a subject in need thereof.

In some embodiments, the method of treatment comprises monitoring the level of at least one biomarker (e.g., microorganism, cancer cell, etc.) during the course of treatment of a disease or disorder. In some embodiments, the method of treatment comprises an assessment of the effectiveness of the treatment regimen for a disease or disorder, such as infection or cancer, by detecting at least one biomarker in an effective amount from samples obtained from a subject over time and comparing the amount of biomarker or biomarkers detected. In some embodiments, a first sample is obtained prior to the subject receiving treatment and one or more subsequent samples are taken after or during treatment of the subject. In some embodiments, changes in the level of at least one biomarker over time provide an indication of effectiveness of the therapy.

To identify therapeutics or drugs that are appropriate for a specific subject, a test sample from the subject can also be exposed to a therapeutic agent or a drug, and the level of one or more biomarkers (e.g., microorganism) can be determined. Biomarker levels can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy.

Additionally, therapeutic agents suitable for administration to a particular subject can be identified by detecting one or more biomarkers in an effective amount from a sample obtained from a subject and exposing the subject-derived sample to a test compound that determines the amount of the biomarker(s) in the subject-derived sample. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

In various exemplary embodiments, effecting a therapy comprises administering a disease-modulating drug to the subject. The subject may be treated with one or more drugs until altered levels of the measured biomarkers return closer to the baseline value measured in a population not having a disease or disorder, or showing improvements in disease biomarkers as a result of treatment with a drug. Additionally, improvements related to a changed level of a biomarker or clinical parameter may be the result of treatment with a disease-modulating drug.

Any drug or any combination of drugs disclosed herein may be administered to a subject to treat a disease. The drugs herein can be formulated in any number of ways, often according to various known formulations in the art or as disclosed or referenced herein.

In various embodiments, any drug or any combination of drugs disclosed herein is not administered to a subject to treat a disease. In these embodiments, the practitioner may refrain from administering the drug or any combination of drugs, may recommend that the subject not be administered the drug or any combination of drugs or may prevent the subject from being administered the drug or any combination of drugs.

In various embodiments, one or more additional drugs may be optionally administered in addition to those that are recommended or have been administered.

### Compositions

The invention also includes compositions comprising the antimicrobial compounds identified by the methods of the invention described herein. The disclosure also encompasses a pharmaceutical composition comprising a compound of the disclosure. In one embodiment, the pharmaceutical composition is useful for inhibiting bacterial infections. In one embodiment, the pharmaceutical composition is useful for overcoming antibacterial resistance. Such a pharmaceutical composition may consist of a compound of the disclosure in a form suitable for administration to a subject. The compound of the disclosure may be present in the pharmaceutical composition in the form of a physiologically acceptable salt, such as in combination with a physiologically acceptable cation, as is well known in the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

In an embodiment, the pharmaceutical compositions useful for practicing the method of the disclosure may be administered to deliver a dose of between 1 ng/kg/day and 100 mg/kg/day (e.g., about 1 ng/kg/day, about 10 ng/kg/day, 100 ng/kg/day, about 500 ng/kg/day, about 1000 ng/kg/day, about 5000 ng/kg/day, about 10000 ng/kg/day, about 50000 ng/kg/day, about 1 mg/kg/day, about 10 mg/kg/day, about 100 mg/kg/day, inclusive of all value sand ranges therebetween). In another embodiment, the pharmaceutical compositions useful for practicing the disclosure may be administered to deliver a dose of between 1 ng/kg/day and 500 mg/kg/day (e.g., about 1 ng/kg/day, about 10 ng/kg/day, 100 ng/kg/day, about 500 ng/kg/day, about 1000 ng/kg/day, about 5000 ng/kg/day, about 10000 ng/kg/day, about 50000 ng/kg/day, about 1 mg/kg/day, about 10 mg/kg/day, about 100 mg/kg/day, about 200 mg/kg/day, about 300 mg/kg/day, about 400 mg/kg/day, or about 500 mg/kg/day inclusive of all value sand ranges therebetween).

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient (e.g., about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, inclusive of all values and subranges therebetween).

Pharmaceutical compositions of the disclosure may be formulated for any suitable route of administration, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include bringing the active ingredient into association with a carrier or one or more other accessory ingredients, and then, if necessary or desirable, shaping or packaging the product into a desired single- or multi-dose unit. The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In one embodiment, the compositions of the disclosure are formulated using one or more pharmaceutically acceptable excipients or carriers. In one embodiment, the pharmaceutical compositions of the disclosure comprise a therapeutically effective amount of a compound of the disclosure and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention or reduction of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; antiseptics; antiviral agents; anticoagulants; stabilizing agents; and pharmaceutically acceptable polymeric or hydrophobic materials. Other “additional ingredients” which may be included in the pharmaceutical compositions of the disclosure are known in the art and described, for example in Genaro, ed. (1985, Remington's Pharmaceutical Sciences, Mack Publishing Co., Easton, Pa.), which is incorporated herein by reference.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition optionally includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

In some embodiments, the pharmaceutical compositions of the present disclosure (e.g., containing therapeutically effective amounts of one or more compounds of Formula (I), (II), and (III) may be formulated as immediate release formulation, a delayed release formulation, or a sustained release formulation, and may comprise at least one pharmaceutically acceptable carrier, diluent, and/or excipient. Pharmaceutically acceptable carriers, diluents or excipients include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, aqueous and non-aqueous solutions. Pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate. Aqueous carriers suitable for use in the present application include, but are not limited to, water, ethanol, alcoholic/aqueous solutions, glycerol, emulsions or suspensions, including saline and buffered media.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. Liquid carriers suitable for use in the present application include, but are not limited to, water (partially containing additives, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

The compositions useful within the disclosure comprise at least one compound of Formula (I), (II), and (III). The compositions of the disclosure may be used in aqueous emulsions such as latexes, water-based paints and coatings, caulks and adhesives, tape joint compounds, mineral slurries, water-cooling systems, personal care products, soaps and detergents, disinfectants, cleaners, and sanitizers, pesticide products, oilfield water and water-based fluids used in oilfield applications including drilling muds, fracturing fluids, and hydrotest fluids, and the like. In one embodiment, the composition is an antimicrobial composition. In one embodiment, the composition is an antiseptic.

Solid carriers suitable for use in the present application include, but are not limited to, inactive substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets may contain up to 99% of the active compound. Suitable solid carriers include, for example, calcium phosphate, magnesium stearate, talc, sugars, lactose, dextrin, starch, gelatin, cellulose, polyvinylpyrrolidone, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide delayed or controlled release of the active ingredient therein using, for example, hydroxypropyl methylcellulose in varying proportions to provide the desired release profile. Tablets may optionally be provided with an enteric coating, to provide release in parts of the gut other than the stomach.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations described herein. Diluents increase the bulk of a solid pharmaceutical composition and/or combination, and may make a pharmaceutical dosage form containing the composition and/or combination easier for the patient and care giver to handle. In various embodiments, diluents for solid compositions include, for example, microcrystalline cellulose (e.g., AVICEL), microtine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc, and/or mixtures of any of the foregoing. Specific examples of: microcrystalline cellulose include those sold under the Trademark Avicel (FMC Corp., Philadelphia, Pa.), for example, Avicel™ pH101, Avicel™ pH102 and Avicel™ pH112; lactose include lactose monohydrate, lactose anhydrous and Pharmatose DCL21; dibasic calcium phosphate includes Emcompress.

Lubricants are used to facilitate tablet manufacture, promoting powder flow and preventing particle capping (i.e., particle breakage) when pressure is relieved. Useful lubricants are magnesium stearate, calcium stearate, stearic acid, glyceryl behenate, talc, colloidal silicon dioxide such as Aerosil™ 200, mineral oil (in PEG), hydrogenated vegetable oil (e.g., comprised of hydrogenated and refined triglycerides of stearic and palmitic acids), combinations thereof.

Binders are used to impart cohesive qualities to a tablet, and thus ensure that the tablet or tablet layer remains intact after compression. Suitable binder materials include, but are not limited to, starch (including corn starch and pregelatinized starch), gelatin, sugars (including sucrose, glucose, dextrose and lactose), polyethylene glycol, polyvinyl alcohol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum, and combinations thereof. Examples of polyvinylpyrrolidone include povidone, copovidone and crospovidone.

Fillers include, for example, materials such as silicon dioxide, titanium dioxide, alumina, talc, kaolin, powdered cellulose, microcrystalline cellulose, urea, sodium chloride, as well as saccharides, or combinations thereof. Any suitable saccharide may be used in the composition of the present invention. As used herein, the “saccharides” used in the invention include sugar alcohols, monosaccharides, disaccharides, and oligosaccharides. Exemplary sugar alcohols include, but not limited to, xylitol, mannitol, sorbitol, erythritol, lactitol, pentitol, and hexitol. Exemplary monosaccharides include, but are not limited to, glucose, fructose, aldose and ketose. Exemplary disaccharides include, but are not limited to, sucrose, isomalt, lactose, trehalose, and maltose. Exemplary oligosaccharides include, but are not limited to, fructo-oligosaccharides, inulin, galacto-ologosaccharides, and mannan-oligosaccharides. In some embodiments, the saccharide is sorbitol, mannitol, or xylitol. In some embodiments, the saccharide is sorbitol. In some embodiments, the saccharide is sucrose.

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Other non-limiting examples of suitable disintegrants include, for example, lightly crosslinked polyvinyl pyrrolidone, corn starch, potato starch, maize starch and modified starches, croscarmellose sodium, crospovidone, sodium starch glycolate, and combinations and mixtures thereof.

In some embodiments of the present disclosure, the pharmaceutical composition may be prepared in an oral formulation. For oral administration, the compounds can be formulated readily by combining the active compounds with pharmaceutically acceptable carriers known in the art. Such carriers enable the compounds disclosed herein to be formulated as tablets, pills, dragees, capsules, liquids, gels, syrups, slurries, suspensions and the like, for oral ingestion by a subject. Pharmaceutical compositions for oral use may be obtained as solid excipients, optionally grinding a resulting mixture, and processing the mixture of granules, after adding suitable adjuvants, if desired, to obtain tablets or dragee cores. Such oral pharmaceutical compositions may also be prepared by milling or melt extrusion. Suitable excipients may be any of those disclosed herein and, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Wetting agents, such as sodium dodecyl sulfate and the like, may be added.

In some embodiments, one or more of the compounds of Formula (I), (II), and (III) are combined with excipients to form a core comprising an active (an active core), thereby forming a solid dosage form. In some embodiments, the active core may comprise an inert particle such as a sugar sphere with an appropriate mean particle size. In one embodiment, the inactive core may be a sugar sphere, a cellulose sphere, a spheroidal silicon dioxide bead, a buffer crystal or an encapsulated buffer crystal, such as calcium carbonate, sodium bicarbonate, fumaric acid, tartaric acid, etc. Buffer crystals are useful to alter the microenvironment. Alternatively, in accordance with other embodiments, drug-containing microgranules or pellets may be prepared by rotogranulation, high-shear granulation and extrusion-spheronization or compression of the drug (as mini-tablets, e.g., having a diameter of about 2 mm or more), a polymeric binder and optionally fillers/diluents.

In some embodiments, dragee cores may be provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds doses.

In some embodiments, pharmaceutical compositions described herein comprise one or more delayed release components. In some embodiments, delayed release is achieved by appropriately coating a drug-containing component with one or more suitable delayed-release polymers (also referred to as a controlled release polymer or rate-controlling polymer) or embedding the drug in a matrix comprising one or more suitable delayed-release polymers. Suitable delayed-release polymers include pharmaceutically acceptable water-insoluble polymers (also referred to as hydrophobic polymers), pharmaceutically acceptable water-soluble polymers (also referred to as hydrophilic polymers), pharmaceutically acceptable gastrosoluble polymers, pharmaceutically acceptable enteric polymers, and combinations thereof.

Non-limiting examples of pharmaceutically acceptable water-insoluble polymers include acrylic polymers, methacrylic acid polymers, acrylic copolymers, such as a methacrylic acid-ethyl acrylate copolymer available under the trade name of EUDRAGIT® (type L, RL, RS and NE30D), and their respective esters, zein, waxes, shellac and hydrogenated vegetable oil, cellulose derivatives, such as ethyl cellulose, cellulose acetate, cellulose acetate butyrate, and the like.

Non-limiting examples of pharmaceutically acceptable water-soluble polymers include homopolymers and copolymers of N-vinyl lactams, including homopolymers and copolymers of N-vinyl pyrrolidone, e.g. polyvinylpyrrolidone (PVP), copolymers of N-vinyl pyrrolidone and vinyl acetate or vinyl propionate, cellulose esters and cellulose ethers, in particular methylcellulose and ethylcellulose, hydroxyalkylcelluloses, in particular hydroxypropylcellulose, hydroxyalkylalkylcelluloses, and hydroxypropylmethylcellulose, cellulose phthalates, succinates, butyrates, or trimellitates, in particular cellulose acetate phthalate, hydroxypropylmethylcellulose phthalate, hydroxypropylmethylcellulose succinate, and hydroxypropylmethylcellulose acetate succinate; high molecular polyalkylene oxides such as polyethylene oxide and polypropylene oxide and copolymers of ethylene oxide and propylene oxide, polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates), polyacrylamides, vinyl acetate polymers such as copolymers of vinyl acetate and crotonic acid, partially hydrolyzed polyvinyl acetate (also referred to as partially saponified “polyvinyl alcohol”), polyvinyl alcohol, polyethylene glycol oligo- and polysaccharides such as carrageenans, galactomannans and xanthan gum, or mixtures of one or more thereof.

Non-limiting examples of gastrosoluble polymers include maltrin, an aminoalkyl methacrylate copolymer available under the trade name of EUDRAGIT® (type E100 or EPO), polyvinylacetal diethylaminoacetate e.g., AEA® available from Sankyo Company Limited, Tokyo (Japan), and the like.

Non-limiting examples of such enteric polymers include carboxymethylethylcellulose, cellulose acetate phthalate (CAP), cellulose acetate succinate, methylcellulose phthalate, hydroxymethylethylcellulose phthalate, hydroxypropylmethylcellulose phthalate (HPMCP), hydroxypropylmethylcellulose acetate succinate (HPMCAS), polyvinyl alcohol phthalate, polyvinyl butyrate phthalate, polyvinyl acetal phthalate (PVAP), a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate, a copolymer of methacrylic acid/methyl methacrylate, cellulose acetate hexahydrophthalate, hydroxypropyl methylcellulose hexahydrophthalate, hydroxypropyl methylcellulose phthalate, cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, SANDARAC, copal collophorium, and combinations comprising one or more of the foregoing polymers. Yet other examples of enteric polymers include synthetic resin bearing carboxyl groups. The term “enteric polymer” as used herein is defined to mean a polymeric substance that when used in an enteric coat formulation, is substantially insoluble and/or substantially stable under acidic conditions at a pH of less than about 5 and which are substantially soluble or can decompose under conditions exhibiting a pH of about 5 or more.

Non-limiting examples of hydrophilic polymers include hydroxypropyl celluloses (HPC), hydroxypropyl methylcelluloses, methylcelluloses, polyethylene oxides, sodium carboxymethyl celluloses, and the like, or combinations thereof.

In certain embodiments, the delayed release component is a matrix. As used herein, the term “matrix” means a composition in which the drug is embedded or dispersed in water soluble, water insoluble, or hydrophilic polymers, or lipophilic maters, in order to achieve delayed release of the drug. The mechanisms of the drug release generally involve drug diffusion through a viscous gel layer or tortuous channels; and/or drug dissolution via gradual erosion or degradation of the polymer(s). In some embodiments, the matrix comprises swellable/erodable polymers, for example hydrophilic polymers which in contact with the water form a gel of high viscosity. In other embodiments, the matrix comprises water-insoluble polymers or lipophilic polymers.

For example, the matrix may be prepared using one or more hydrophilic polymers (e.g., hydroxypropyl cellulose, hydroxypropyl methylcellulose, polyethylene oxide), one or more lipophilic materials (e.g., carnauba wax, hardened castor oil, hardened rape seed oil, polyglycerin fatty acid ester), and/or coating tablets or granules with one or more delayed release polymers (e.g., cellulose polymers such as ethylcellulose; acrylic acid copolymer such as aminoalkyl methacrylate copolymer RS [Eudragit RS (trade name, Degussa Co.)], ethyl acrylate-methyl methacrylate copolymer suspension [Eudragit NE (trade name, Degussa Co.)]).

The hydrophilic matrix may further contain a pH-dependent polymer. The term “pH-dependent” refers to a polymer which releases the active at a certain pH. Non-limiting examples of suitable pH-dependent polymers include hydroxypropyl methylcellulose phthalate, cellulose acetate phthalate, carboxymethyl ethyl cellulose, methyl methacrylate-methacrylic acid copolymer, methacrylic acid-ethyl acrylate copolymer, ethyl acrylate-methyl methacrylate-trimethylammoniumethyl methacrylate chloride copolymer, methyl methacrylate-ethyl acrylate copolymer, methacrylic acid-methyl acrylate-methyl methacrylate copolymer, hydroxypropyl cellulose acetate succinate, polyvinyl acetate phthalate and the like, and combinations thereof.

In some embodiments, the pharmaceutical composition is formulated as a sustained release formulations, e.g., by appropriately integrating additional polymers into the composition, or as coatings over the core (e.g., pellet or granule). The polymers useful for this purpose can be, but are not limited to, ethylcellulose; hydroxypropylmethylcellulose; hydroxypropylcellulose; hydroxyethylcellulose; carboxymethylcellulose; methylcellulose; nitrocellulose; Eudragit R; Eudragit RS; and Eudragit RL; Carbopol; polyethyleneoxide or polyethylene glycols with molecular weights in excess of 8,000 daltons. In some embodiments, these polymers are present concentrations from about 4-20 w/w % (e.g., about 4, about 5, about 6, about 7, about 8, about 9, about 10, about 11, about 12, about 13, about 14, about 15, about 16, about 17, about 18, about 19, or about 20% w/w %). The sustained release polymers may be combined with the delayed release components described above.

The compositions useful within the disclosure may further comprise at least one additional antimicrobial agent. Non-limiting examples of the at least one additional antimicrobial agent are levofloxacin, doxycycline, neomycin, clindamycin, minocycline, gentamycin, rifampin, chlorhexidine, chloroxylenol, methylisothizolone, thymol, α-terpineol, cetylpyridinium chloride, hexachlorophene, triclosan, nitrofurantoin, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinafloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

In one embodiment, the compound of the disclosure and the at least one additional antimicrobial agent act synergistically in preventing, reducing or treating bacterial infections. A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

The pharmaceutical compositions may be prepared by any suitable method, such as mixing, dissolving, granulating, dragee-making, levigating, emulsifying, encapsulating, entrapping, or lyophilizing processes.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation involves the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

The methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Antibacterial Syn-BNPs with Diverse Mechanisms of Action

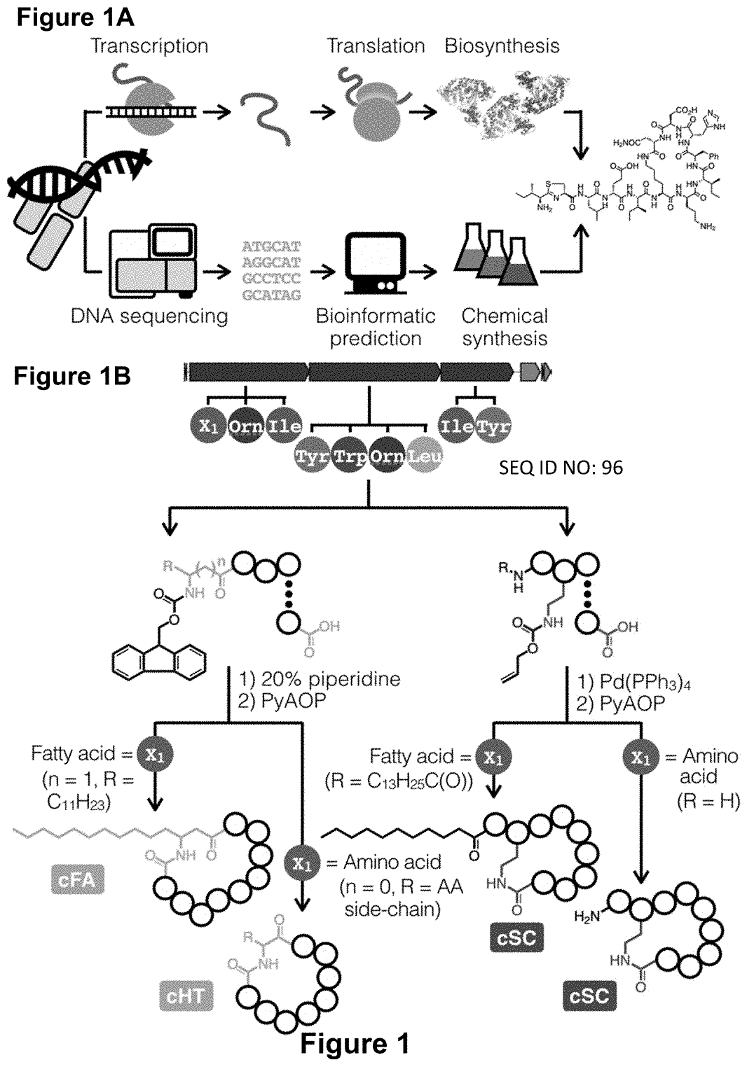

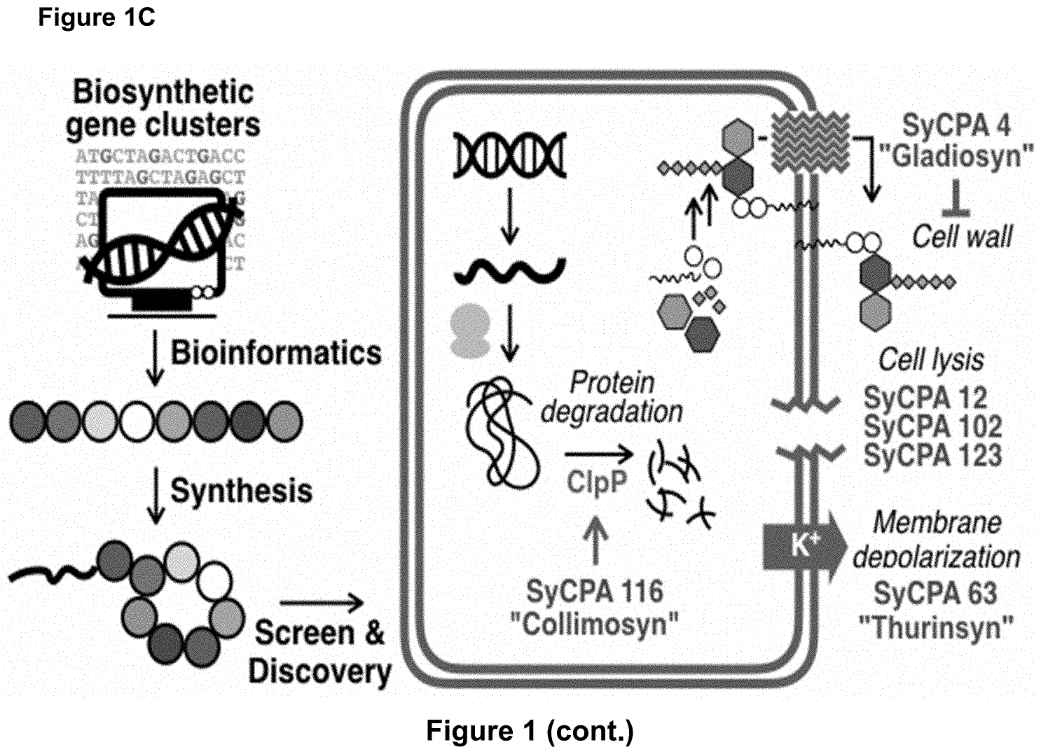

In order to development of new methods for converting genetic information into new antibacterial molecules that function by diverse modes of action, a pipeline that does not rely on the complex in vivo processes of transcription, translation, and enzyme catalysis to produce natural products for sequenced gene clusters has been explored (FIG. 1A). In this purely in vitro approach, which was termed syn-BNPs, the structures of natural products encoded by biosynthetic gene clusters were predicted bioinformatically, and the resulting predictions were then chemically synthesized to generate libraries of molecules that were inspired by nature (Chu J et al., 2016, Nat. Chem. Biol., 12:1004-1006; Vila-Farres X et al., 2017, J. Am. Chem. Soc., 139:1404-1407; Vila-Farres X et al., 2018, mSphere 3; Chu Jet al., 2018, ACS Infect. Dis., 4:33-38; Chu Jet al., 2019, J. Am. Chem. Soc., 141:15737-15741). The syn-BNP approach should not generate exact copies of natural products but instead provided libraries of biomimetic natural product congeners that should be enriched for evolutionarily selected biological activities.

Although bacteria use numerous biosynthetic strategies to generate structurally diverse antibiotics, among the most productive is the ribosome-independent biosynthesis of short linear and cyclic peptides by nonribosomal peptide synthetases (NRPSs) (Fischbach M A et al., 2006, Chem. Rev., 106:3468-3496). Algorithms for predicting the peptide products of NRPS gene clusters as well as methods for synthesizing peptides are well-established, making these gene clusters appealing targets for syn-BNP discovery efforts.

Although the majority of characterized nonribosomal peptides are cyclic, early syn-BNP studies largely focused on the synthesis of simple linear peptides (Chu J et al., 2016, Nat. Chem. Biol., 12:1004-1006; Vila-Farres X et al., 2017, J. Am. Chem. Soc., 139:1404-1407; Vila-Farres X et al., 2018, mSphere 3; Chu J et al., 2018, ACS Infect. Dis., 4:33-38). To better mimic natural products, Example 1 discloses expanding the predictive and synthetic efforts to include NRPS-inspired cyclic structures (Chu Jet al., 2019, J. Am. Chem. Soc., 141:15737-15741). Example 1 also discloses the discovery of nine new syCPAs that collectively: had multiple modes of actions, showed both narrow and broad spectra of activity against antibiotic-resistant pathogens, and, in many cases, did not easily develop resistance in the laboratory. The results suggested that the application of increasingly elaborate syn-BNP methods to cryptic biosynthetic gene clusters was likely to be a rewarding strategy for identifying naturally inspired, bioactive molecules—in particular, mechanistically diverse antibiotics that could help repopulate and diversify antibiotic discovery pipelines.

### Bioinformatic Analysis, Design, and Synthesis of NRPS-Inspired Cyclic Syn-BNPs

Nonribosomal peptides were produced by modular assembly line-like enzymes. In canonical systems, each module incorporated a single amino acid into a growing peptide. Detailed bioinformatic analyses of functionally characterized NRPS gene clusters have led to the development of algorithms that use protein sequence data to predict the identity and order of the amino acid building blocks encoded by a gene cluster (Stachelhaus T et al., 1999, Chem. Biol., 6:493-505; Minowa Yet al., 2007, J. Mol. Biol., 368:1500-1517; Rottig Metal., 2011, Nucleic Acids Res., 39:W362-367; Blin K et al., 2013, Nucleic Acids Res., 41:W204-212). To generate syn-BNP targets, large (>5 adenylation domains) NRPS gene clusters found in 3,000 complete bacterial genomes present in GenBank, were bioinformatically analyzed (Genome List—Genome—NCBI, accessed, December, 2014: ncbi.nlm.nih.gov/genome/browse #!/overview/). For each gene cluster the peptide, which it encoded, was predicted using three different prediction tools (Stachelhaus T et al., 1999, Chem. Biol., 6:493-505; Minowa Y et al., 2007, J. Mol. Biol., 368:1500-1517; Rottig M et al., 2011, Nucleic Acids Res., 39:W362-367). Any gene clusters that were predicted to encode known nonribosomal peptides, a large number of tailoring enzymes, or residues that did not have a consensus prediction across algorithms, were excluded from further analysis. Those gene clusters that did not appear to follow a canonical colinear organization pattern were also removed from further analysis. From the remaining NRPS gene clusters, 96 linear peptide predictions were selected to serve as the basis of the cyclic syn-BNP targets (Table 1).

TABLE 1
List of Syn-BNP Peptides.

| No | Pred. seq.ª | NRPS gene cluster origin | Ref. genome^{b} | Locus (nt)^{c} |
|---|---|---|---|---|
| 1 | X₁zSASLeE | Bacillus cereus B4264 | NC_011725.1 | 2,341,208-2,406,996 |
| 2 | X₁zpqAvFP | Burkholderia gladioli BSR3, chr 2 | NC_015376.1 | 1,312,845-1,376,223 |
| 3 | X₁ZTyVS | Burkholderia gladioli BSR3, chr 2 | NC_015376.1 | 2,023,222-2,080,171 |
| 4 | X₁AZsKrKGv | Burkholderia gladioli BSR3, chr 2 | NC_015376.1 | 2,103,056-2,186,245 |
| 5 | X₁zOSEtLBTTOGV | Burkholderia pseudomallei K96243, chr 2 | NC_006350.1 | 2,187,213-2,268,799 |
| 6 | X₁zESRtLBTTeGV | Burkholderia pseudomallei K96243, chr 2 | NC_006350.1 | 2,187,213-2,268,799 |
| 7 | X₁LZyaAaV | Burkholderia rhizoxinica HKI 454, pBRH01 | NC_014718.1 | 3,955-66,916 |
| 8 | X₁nZISeVlfL | Clostridium botulinum A2 str. Kyoto | NC_012563.1 | 338,515-416,384 |
| 9 | X₁BTYSKEYFF | Clostridium cellulovorans p743B | NC_014393.1 | 2,914,102-2,963,633 |
| 10 | X₁BsBASVDIF | Clostridium cellulovorans p743B | NC_014393.1 | 2,967,242-3,025,608 |
| 11 | X₁LIZFAY | Rhodococcus pyridinivorans SB3094 | NC_023150.1 | 2,309,268-2,368,050 |
| 12 | X₁znlOsPofT | Dickeya dadantii Ech586 | NC_013592.1 | 3,414,889-3,485,467 |
| 13 | X₁dZDtSN | Herbaspirillum seropedicae SmR1 | NC_014323.1 | 2,631,343-2,698,825 |
| 14 | X₁GZATFGVF | Mycobacterium marinum M | NC_010612.1 | 992,526-1,058,784 |
| 15 | X₁AZTAGTFTT | Mycobacterium marinum M | NC_010612.1 | 3,964,578-4,014,848 |
| 16 | X₁AZTTTTTrDVTR | Mycobacterium rhodesiae NBB3 | NC_016604.1 | 4,719,612-4,799,700 |
| 17 | X₁ZSSTSSSSSSSWS | Nocardia cyriacigeorgica GUH-2 | NC_016887.1 | 5,347,951-5,451,235 |
| 18 | X₁zToDGndGYVOF | Paenibacillus mucilaginosus KNP414 | NC_015690.1 | 3,128,888-3,215,024 |
| 19 | X₁ZCiFBCO | Paenibacillus mucilaginosus KNP414 | NC_015690.1 | 5,527,457-5,599,870 |
| 20 | X₁ZvFtnA | Paenibacillus polymyxa E681 | NC_014483.1 | 61,938-131,860 |
| 21 | IfKSTf | Paenibacillus terrae HPL-003 | NC_016641.1 | 1,241,438-1,303,692 |
| 22 | X₁voGsfSPQP | Paenibacillus terrae HPL-003 | NC_016641.1 | 4,207,798-4,291,196 |
| 23 | X₁aVLdGGZTsk | Pseudomonas entomophila L48 | NC_008027.1 | 3,437,580-3,517,142 |
| 24 | X₁LZDVAS | Pseudomonas syringae pv. syringae B728a | NC_007005.1 | 4,391,388-4,451,295 |
| 25 | X₁lZFLSyY | Rhodococcus opacus B4 | NC_012522.1 | 3,545,422-3,611,002 |
| 26 | X₁WZGYR | Rhodococcus opacus B4 | NC_012522.1 | 5,997,386-6,053,147 |
| 27 | X₁FZAtFF | Rhodococcus pyridinivorans SB3094 | NC_023150.1 | 859,328-921,377 |
| 28 | X₁YZAKR | Gordonia sp. KTR9 | NC_018581.1 | 1,029,407-1,089,317 |
| 29 | X₁KFBNAVQ | Xanthomonas albilineans GPE PC73 | NC_013722.1 | 836,222-899,513 |
| 30 | X₁GOTnYNySnDNV | Xanthomonas albilineans GPE PC73 | NC_013722.1 | 1,166,681-1,259,075 |
| 31 | X₁AZS*OP | Uncultured bacterium contig 00137 | JX827803.1 | 1-35,392 |
| 32 | X₁DZVEDDGdVP | Uncultured bacterium | n/a | 33,345-119,152 |
| 33 | X₁DYvODDGdVP | Uncultured bacterium | n/a | 33,345-119,152 |
| 34 | X₁ZdSos | Achromobacter xylosoxidans A8 | NC_014640.1 | 2,723,596-2,795,344 |
| 35 | X₁YZYtFF | Amycolicicoccus subflacus DQS3-9A1 | NC_015564.1 | 4,121,859-4,184,019 |
| 36 | X₁vVZSG*vV*S | Burkholderia rhizoxinica HKI 454 pBRH01 | NC_014718.1 | 621,904-691,902 |
| 37 | X₁zEeGV | Burkholderia pseudomallei 668 chr.Il | NC_009075.1 | 2,200,921-2,258,941 |
| 38 | X₁iZtFtV | Streptosporangium roseum DMS 43021 | NC_013595.1 | 7,175,764-7,243,639 |
| 39 | X₁oTVEdG | Burkholderia sp. Y123 chr.3 | NC_016590.1 | 965,645-1,034,316 |
| 40 | X₁lzVSI | Collimonas fungivorans Ter331 | NC_015856.1 | 291,223-348,100 |
| 41 | X₁nZESACLv | Clostridium botulinum H04402 065 | NC_017299.1 | 333,496-408,715 |
| 42 | X₁lZFLSEyG | Rhodococcus erythropolis CCM2595 | NC_022115.1 | 3,424,162-3,472,802 |
| 43 | X₁ZVFTTySS | Rhodococcus erythropolis PR4 DNA | NC_012490.1 | 3,641,373-3,724,302 |
| 44 | X₁YZGYA | Rhodococcus erythropolis PR4 DNA | NC_012490.1 | 3,772,211-3,828,404 |
| 45 | X₁YZlYtIV | Rhodococcus jostii RHA1 | NC_008268.1 | 124,521-171,307 |
| 46 | X₁WEzFtIF | Rhodococcus jostii RHA1 | NC_008268.1 | 257,548-324,163 |
| 47 | X₁FZtRF | Rhodococcus jostii RHA1 | NC_008268.1 | 5,421,621-5,460,424 |
| 48 | X₁KQVAIEAAV | Corallococcus coralloides DSM 2259 | NC_017030.1 | 3,158,299-3,187,644 |
| 49 | X₁lZFLSyW | Rhodococcus opacus B4 | NC_012522.1 | 3,545,422-3,611,002 |
| 50 | X₁oSVdrTGIiAAIL | Teredinibacter turnerae T7901 | NC_012997.1 | 2,500,801-2,571,480 |
| 51 | X₁kGGSD | Thermobifida fusca YX | NC_007333.1 | 2,152,724-2,214,837 |
| 52 | X₁LZyPtIF | Rhodococcus jostii RHA1 | NC_008268.1 | 5,374,757-5,421,456 |
| 53 | X₁VZvTSYY | Burkholderia rhizoxinica HKI 454 | NC_014722.1 | 1,815,672-1,884,887 |
| 54 | X₁dBGGKGD | Marinomonas mediterranea MMB-1 | NC_015276.1 | 1,028,535-1,099,973 |
| 55 | X₁LBDSsdBD | Pseudomonas brassicacearum NFM421 | NC_015379.1 | 2,934,038-3,009,838 |
| 56 | X₁OYVPGTiGyVD | Paenibacillus mucilaginosus KNP414 | NC_015690.1 | 4,432,091-4,514,260 |
| 57 | fAfVyFvzVfVtFv | Paenibacillus mucilaginosus KNP414 | NC_005126.1 | 3,104,571-3,193,674 |
| 58 | X₁iZtWtV | Streptosporangium roseum DSM 43021 | NC_013595.1 | 7,175,764-7,243,639 |
| 59 | X₁TZLllLS | Xenorhabdus bovienii SS-2004 | NC_013892.1 | 739,318-804,275 |
| 60 | ZLrLkrG | Xenorhabdus bovienii SS-2004 | NC_013892.1 | 2,095,493-2,159,645 |
| 61 | X₁DzQTAaoQ | Pseudomonas protegens Pf-5 | NC_004129.6 | 4,705,296-4,776,269 |
| 62 | X₁ZPYOySY | Actinosynnema mirum DSM 43827 | NC_013093.1 | 5,416,139-5,503,748 |
| 63 | X₁OrIRL | Bacillus thuringiensis plasmid BMB171 | NC_014172.1 | 151,784-215,426 |
| 64 | X₁bsvtvL | Chromobacterium violaceum ATCC 12472 | NC_005085.1 | 3,016,595-3,076,834 |
| 65 | X₁nZLLOAL | Frankia alni ACN14a | NC_008278.1 | 4,470,422-4,555,531 |
| 66 | X₁ZTvFtnA | Paenibacillus polymyxa E681 | NC_014483.1 | 61,938-131,860 |
| 67 | X₁YzSsSis | Paenibacillus polymyxa SC2 | NC_014622.1 | 1,071,648-1,147,733 |
| 68 | X₁foVIFAF | Paenibacillus mucilaginosus KNP414 | NC_015690.1 | 4,920,286-5,002,941 |
| 69 | X₁OYVPGyGVD | Paenibacillus mucilaginosus 3016 | NC_016935.1 | 4,198,074-4,268,990 |
| 70 | X₁AZaLtLW | Rhodococcus equi 103S | NC_014659.1 | 4,503,597-4,577,253 |
| 71 | X₁ZnwdwssyVV | Streptomyces griseus NBRC 13350 | NC_010572.1 | 3,747,228-3,843,604 |
| 72 | X₁ZYTGWPwGyN | Streptomyces violaceusniger Tu 4113 | NC_015957.1 | 10,111,293-10,191,037 |
| 73 | X₁kSVAG | Streptomyces collinus Tu 365 | NC_021985.1 | 341,018-489,943 |
| 74 | X₁KFBNAVQ | Xanthomonas albilineans GPE PC73 | NC_013722.1 | 836,222-899,513 |
| 75 | X₁lzavvAsLltsSVL | Bradyrhizobium sp. BTAi1 | NC_009485.1 | 7,072,456-7,158,452 |
| 76 | X₁AdGdZsdG | Azospirillum sp. B510 pAB510a | NC_013855.1 | 962,998-1,053,897 |
| 77 | LvVlAvAvVvWIR | Brevibacillus brevis NBRC 100599 | NC_012491.1 | 3,002,466-3,091,712 |
| 78 | PTWSSTSWSWS | Nocardia cyriacigeorgica GUH-2 | NC_016887.1 | 5,347,951-5,451,235 |
| 79 | fasT*L | Burkholderia sp. YI23 | NC_016625.1 | 87,366-144,166 |
| 80 | DNYFySR | Streptomyces cattleya NRRL 8057 | NC_016111.1 | 2,784,016-2,856,738 |
| 81 | X₃LLVBS | Burkholderia pseudomallei K96243 | NC_006350.1 | 1,978,620-2,037,444 |
| 82 | X₃dGfGeeGE | Bradyrhizobium sp. BTAi1 | NC_009485.1 | 1,110,518-1,183,121 |
| 83 | X₃GQKAGS | Myxococcus stipitatus DSM 14675 | NC_020126.1 | 7,943,455-8,003,631 |
| 84 | X₃bsvtVL | Bacillus subtilis subsp. subtilis str. 168 | NC_000964.3 | 356,968-422,359 |
| 85 | X₃AAFVDFYNT | Corallococcus coralloides DSM 2259 | NC_017030.1 | 4,855,502-4,927,016 |
| 86 | X₃dFyVyVNT | Myxococcus stipitatus DSM 14675 | NC_020126.1 | 5,568,702-5,644,506 |
| 87 | X₃GQVAGS | Myxococcus stipitatus DSM 14675 | NC_020126.1 | 7,943,455-8,003,631 |
| 88 | X₃nvltlV | Xanthomonas oryzae pv. oryzicola BLS256 | NC_017267.1 | 2,606,212-2,667,348 |
| 89 | X₃iVfFFS | Amycolicicoccus subflavus DQS3-9A | NC_015564.1 | 4,533,351-4,595,796 |
| 90 | X₃evVEQ | Burkholderia rhizoxinica HKI 454 | NC_014722.1 | 1,961,984-2,037,081 |
| 91 | X₃VNSQQ | Actinoplanes missouriensis 431 | NC_017093.1 | 5,221,571-5,289,542 |
| 92 | X₃lSLSQ | Bacillus subtilis subsp. subtilis str. 168 | NC_008268.1 | 5,800,513-5,900,006 |
| 93 | X₃ANTNN | Streptomyces avermitilis MA-4680 | NC_003155.4 | 4,480,084-4,546,900 |
| 94 | X₃NGLETF | Kitasatospora setae KM-6054 | NC_016109.1 | 6,477,303-6,546,097 |
| 95 | X₃dEGGdGD | Marinomonas mediterranea MMB-1 | NC_015276.1 | 1,028,535-1,099,973 |

ªAbbreviations for amino acids (AA); in addition to the standard one-letter codes for the 20 canonical amino acids, the following abbreviations were used: 2,3-diaminopropionic acid (Z), 2,4-diaminobutyric acid (B), ornithine (O), asterisk (*) denotes Nα-methylation. Upper and lower cases indicate L and D amino acids, respectively. Abbreviations for N-terminal modifications: tetradecanoic acid or 3-aminotetradecanoic acid (X₃). Residues serving as the site for chemical cyclization are highlighted in bold. For cSC type peptides, a building block with an Alloc protected nucleophilic side-chain was used to replace the predicted AA at the cyclization site during SPPS.
^{b}GenBank accession number.
^{c}Nucleotide range of the NRPS gene cluster that led to the prediction of the Syn-BNP peptide.

The majority of characterized nonribosomal peptides were cyclized through their C-termini, and therefore the current synthetic efforts were focused on generating C-terminally cyclized syn-BNPs. Monocyclic nonribosomal peptides that were cyclized through the C-terminal carboxylate can largely be grouped into three distinct categories based on the nucleophile that was used in the cyclization reaction. The carboxylate can react with either an amino acid side-chain, the N-terminus of the peptide, or in the case of N-acylated peptides, a nucleophile on the fatty acid. While bioinformatically predicting the specific nucleophile used for cyclization remains challenging (Horsman M E et al., 2016, Nat. Prod. Rep., 33:183-202), an analysis of characterized cyclic nonribosomal peptides revealed the following trends (Chu J et al., 2019, J. Am. Chem. Soc., 141:15737-15741; Dictionary of Natural Products, accessed March, 2020: dnp.chemnetbase.com/faces/chemical/ChemicalSearch.xhtml; Medema M H et al., 2015, Nat. Chem. Biol., 11:625-631):
1) when more than one potential side-chain nucleophile was present, nonribosomal peptides were most often cyclized through the residue that yielded the largest macrocycle,
2) fatty acid cyclization most commonly occurred through beta-oxidation of the lipid, and
3) the vast majority of nonribosomal peptide macrocycles contained four or more amino acid residues (n≥4).

These observations were used to guide the design of sets of differential cyclic syn-BNP targets for each linear nonribosomal peptide prediction (FIG. 1B). Specifically, when a nonribosomal peptide was predicted to be N-acylated with a fatty acid, the corresponding syn-BNP peptide was cyclized through either the β-heteroatom of the fatty acid (cFA, FIG. 1B) or the nucleophilic amino acid side-chain closest to the N-terminus (cSC, FIG. 1B). Alternatively, when no fatty acid was predicted to be present at the N-terminus, the syn-BNP peptide was cyclized either head-to-tail (cHT, FIG. 1B) or through a nucleophilic side-chain (cSC, FIG. 1B). Finally, peptides inspired by NRPS gene clusters that were predicted to encode products without a nucleophilic side-chain capable of forming a macrocycle of at least four amino acids were only cyclized either head-to-tail or through their fatty acids. Using this general scheme, the synthesis of 171 syn-BNPs, which were inspired by the 96 linear peptide predictions, were targeted. Each cyclization reaction was partitioned over a C-18 solid-phase extraction cartridge to obtain a semi-pure synthetic cyclic peptide. The desired target mass was identified in 157 (92%) cases (Table 1). Fractions containing the target masses were dried in vacuo, resuspended in DMSO and used directly for bioactivity screening.

### Syn-BNP Screening for Antibiosis

Nonribosomal peptides displayed a wide variety of bioactivities but they have offered their greatest utility as antibiotics (Walsh C T, 2004, Science 303:1805-1810). To identify antibacterial active syn-BNPs, each fraction, containing the mass of a target syn-BNP cyclic peptide against Bacillus subtilis, Escherichia coli, and the ESKAPE pathogens, was screened. ESKAPE pathogens represented the bacteria most commonly associated with antibiotic-resistant nosocomial infections: Enterococcus faecium, Staphylococcus aureus, Klebsiella pneumoniae, Acinetobacter baumannii, Pseudomonas aeruginosa, and Enterobacter cloacae (Boucher H W et al., 2009, Clin. Infect. Dis., 48:1-12).

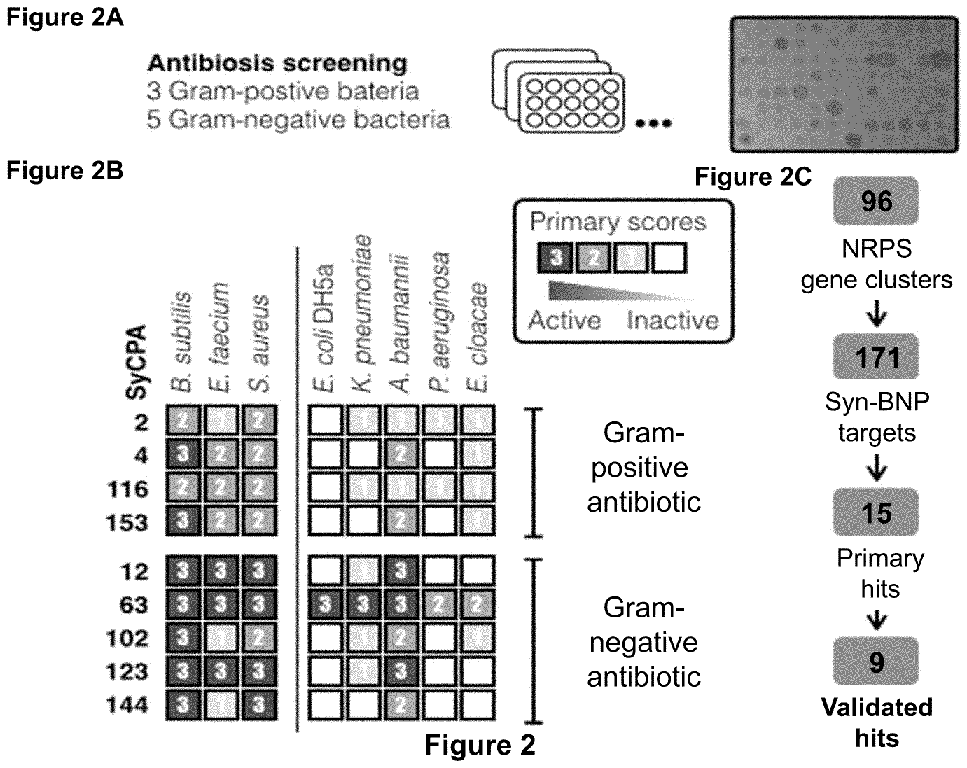

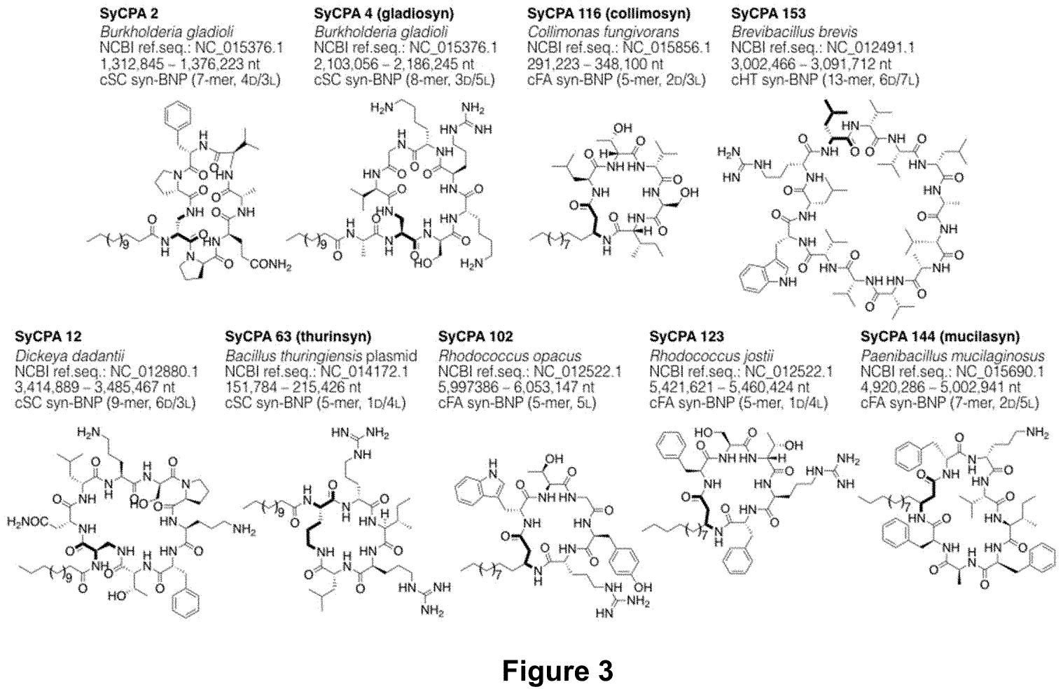

In the initial screen, semi-pure peptides were spotted on solid growth media seeded with each bacterium. After the bacterial lawns matured, a semi-quantitative score on a scale from 0 (inactive) to 3 (clear growth inhibition zone) was assigned to each cyclic peptide (FIG. 2A). Syn-BNPs scoring a “3” against any bacterium or having a combined score of 8 or higher across all pathogens were regarded as primary hits. In total, 15 fractions that reached one or both thresholds were identified. Syn-BNPs were purified by HPLC from the fractions associated with each primary hit and then re-assayed for antibacterial activity (FIG. 2B). Once purified, nine primary hits showed an MIC of ≤8 μg/mL against at least one bacterium in the panel. These validated hits were named SyCPAs and were considered the final set of active compounds (FIG. 3). Many previously characterized natural product antibiotics have similar single-digit μg/mL MICs, indicating that even though syn-BNPs were unlikely to be perfect copies of evolutionary optimized natural products, they can have potencies that were comparable to antibiotics produced using biosynthetic machinery.

### Spectrum of Activity of SyCPAs

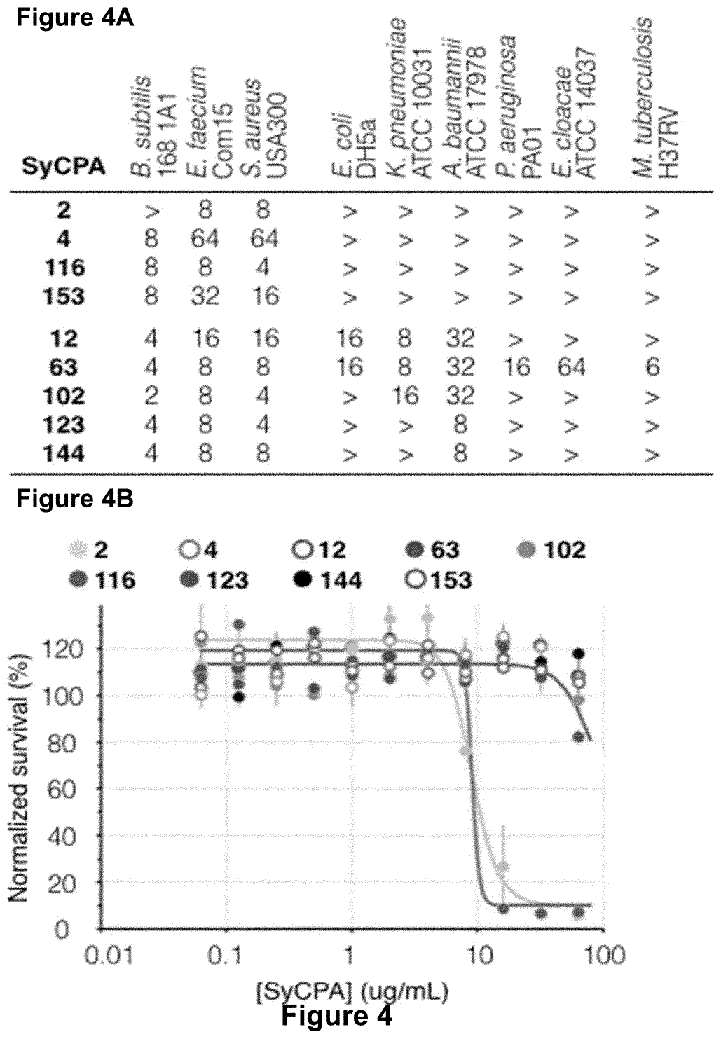

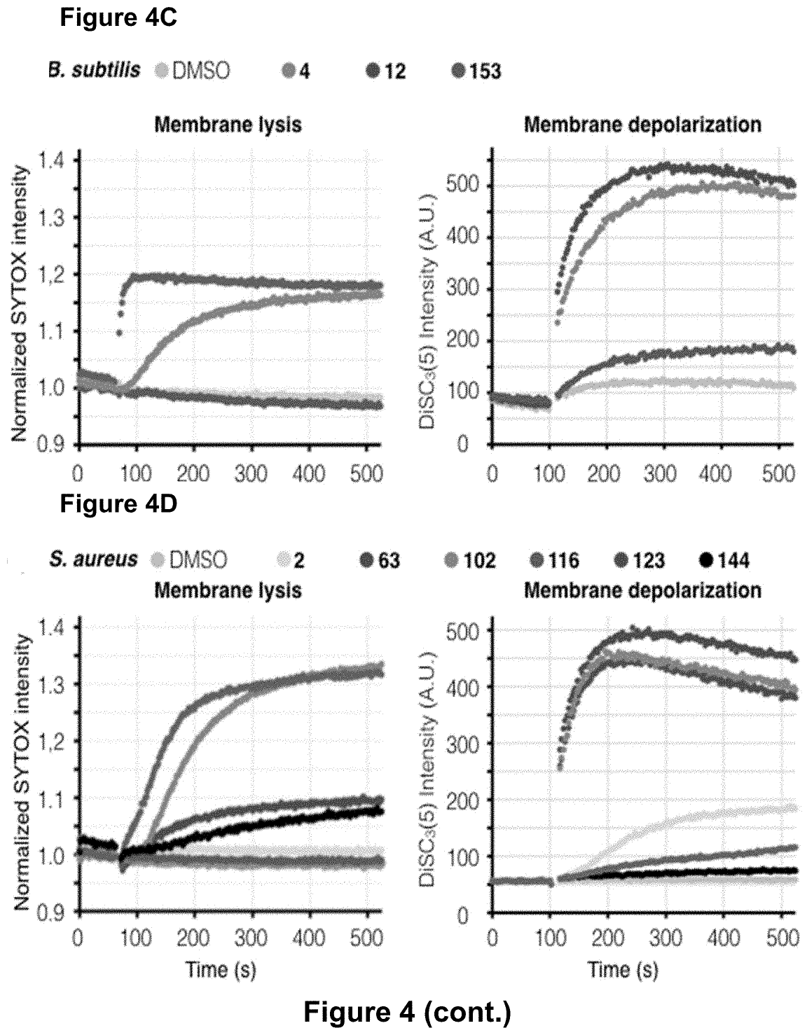

Four SyCPAs were Gram-positive specific antibiotics and five showed activity against at least one Gram-negative bacterium (FIG. 4A). Almost all SyCPAs were active against at least one antibiotic-resistant ESKAPE pathogen. Among the Gram-positive active antibiotics, distinct activity patterns were observed. SyCPA 4 and SyCPA 153 were mostly active against B. subtilis, while SyCPA 2 and SyCPA 116 showed broader Gram-positive activity. Broad-spectrum SyCPAs ranged from being active against a number of the Gram-negative bacteria that were tested (SyCPA 63) to only being active against A. baumannii (SyCPA 123 and SyCPA 144). When assayed for cytotoxicity against HeLa cells using the 3-(4,5-dimethylthiazol-2-yl)-2,5-diphenyl-tetrazolium (MTT) metabolic activity assay (FIG. 4B), SyCPA 2 and SyCPA 116 were the only peptides that showed significant toxicity (IC₅₀ 16 and 11 μg/mL, respectively), indicating that most SyCPAs were not general cytotoxins but were instead specifically antibiotics.

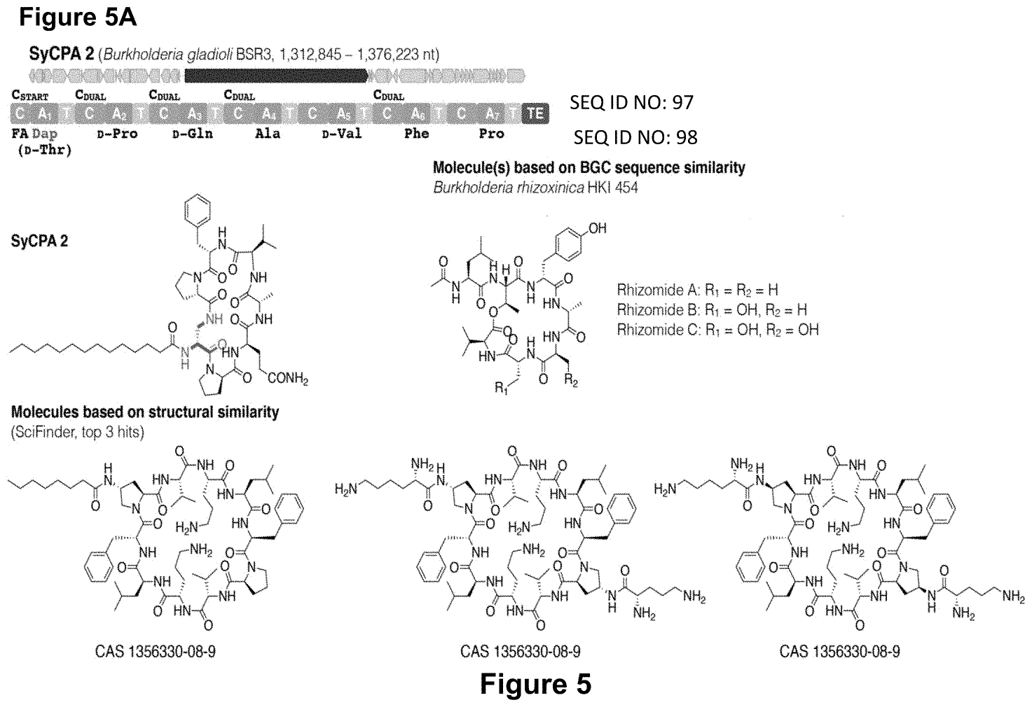

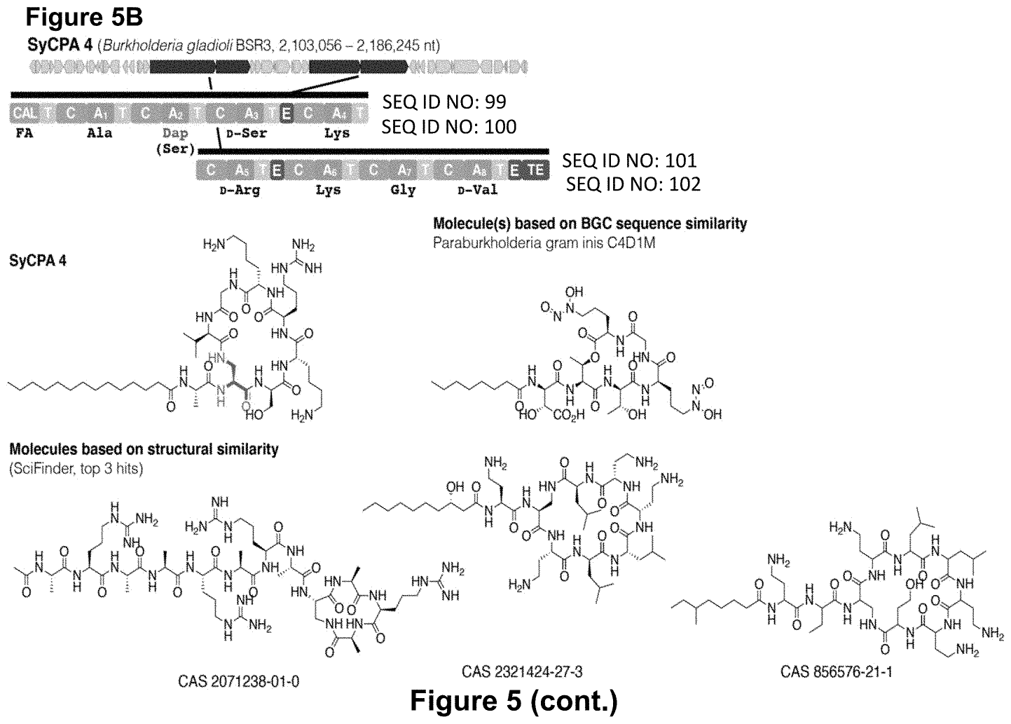

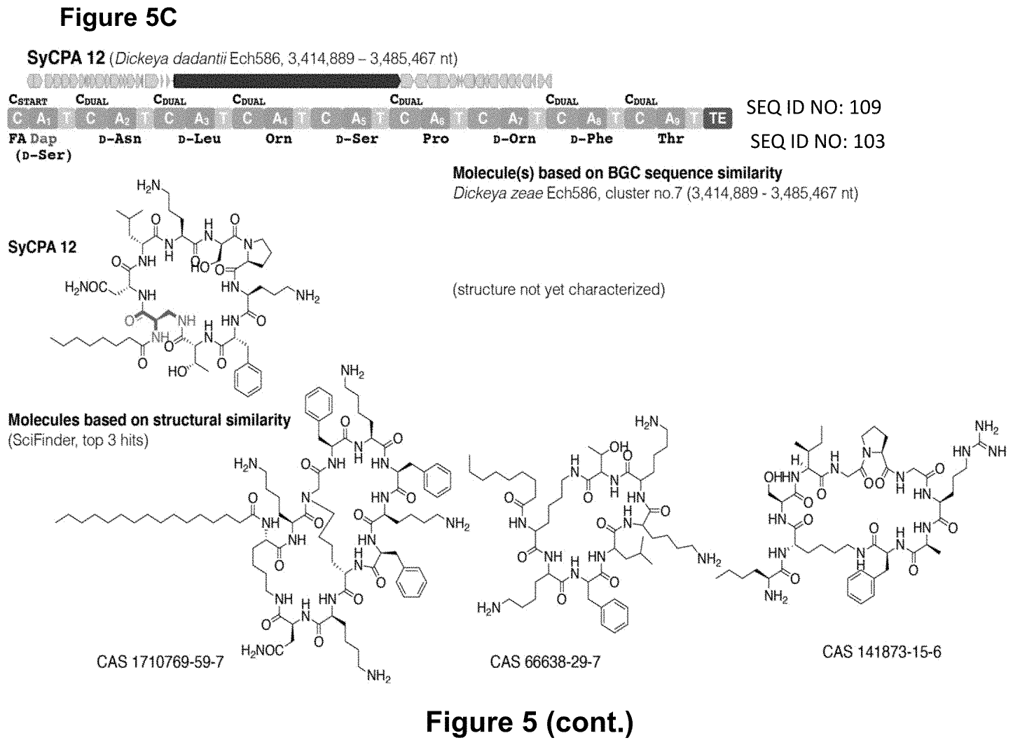

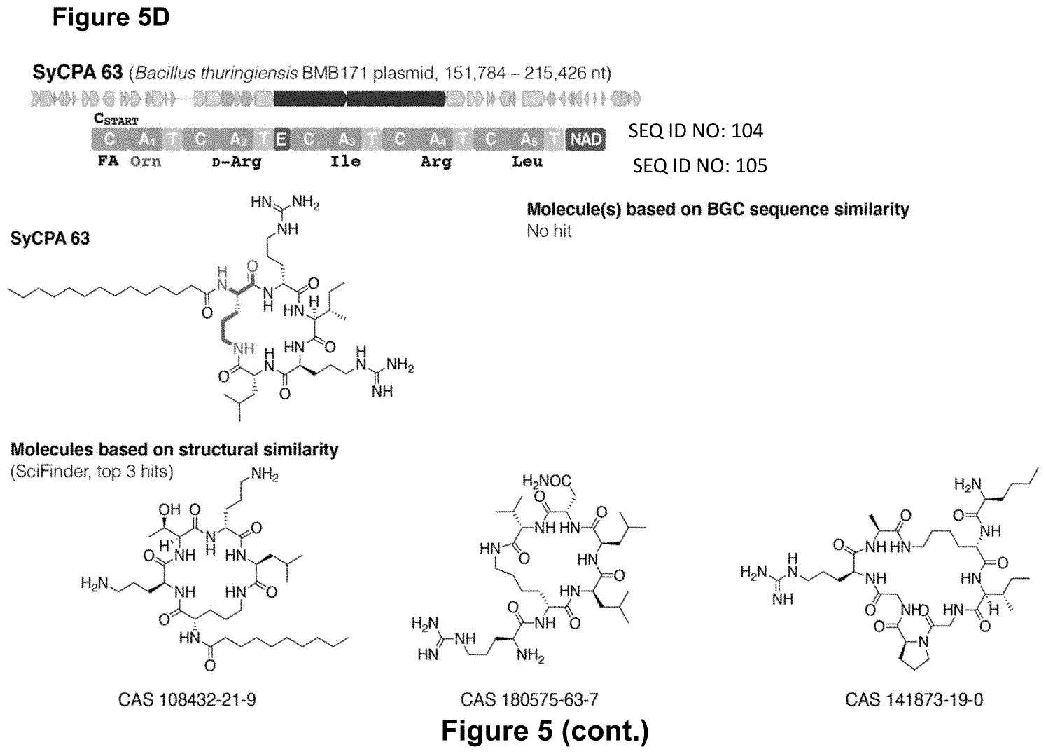

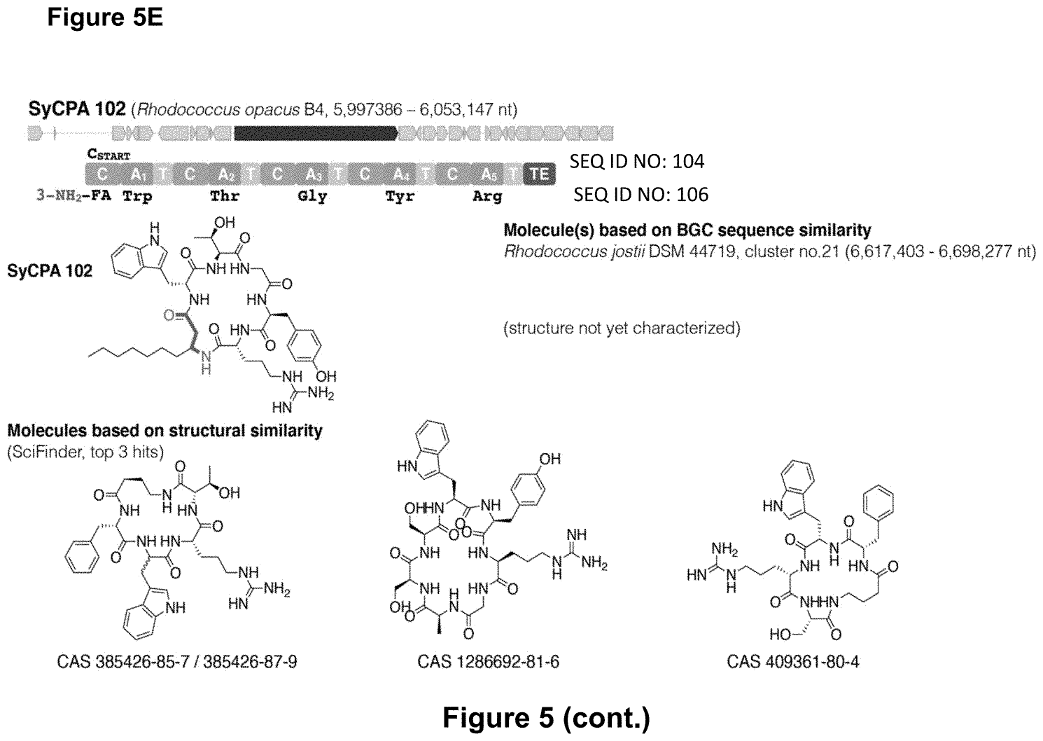

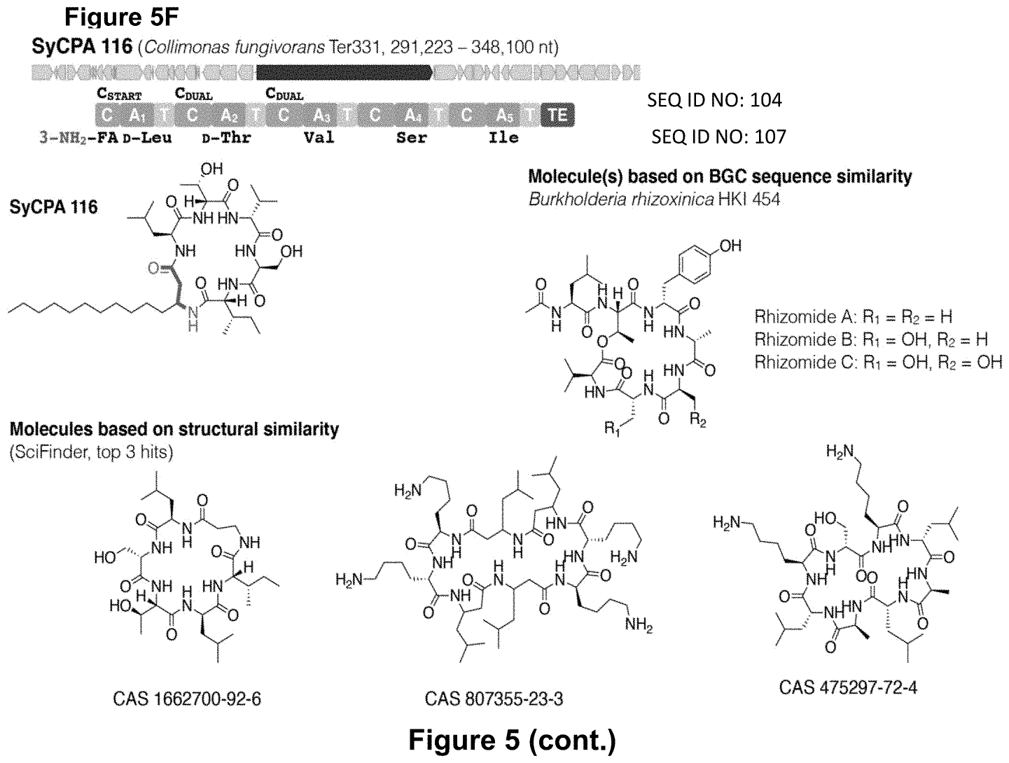

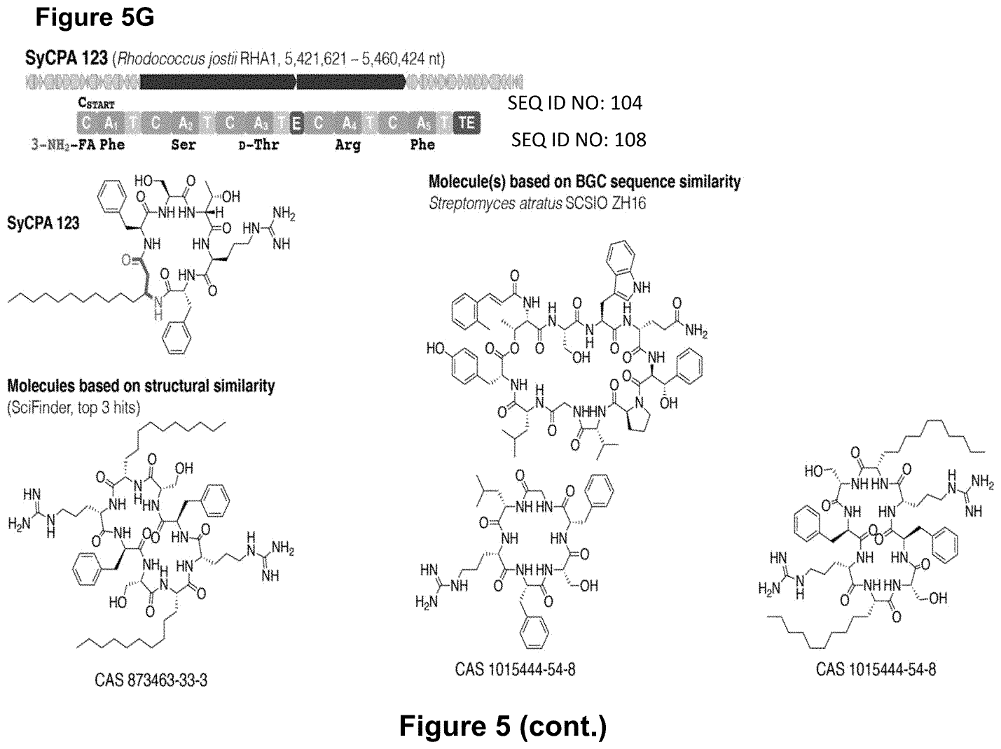

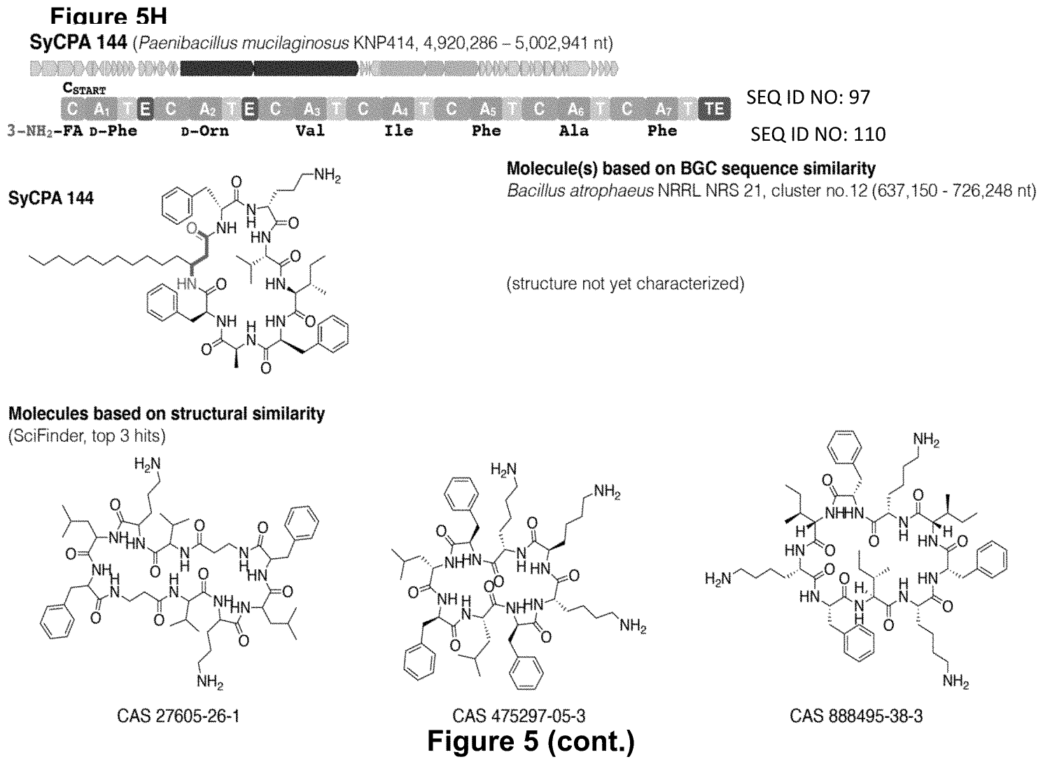

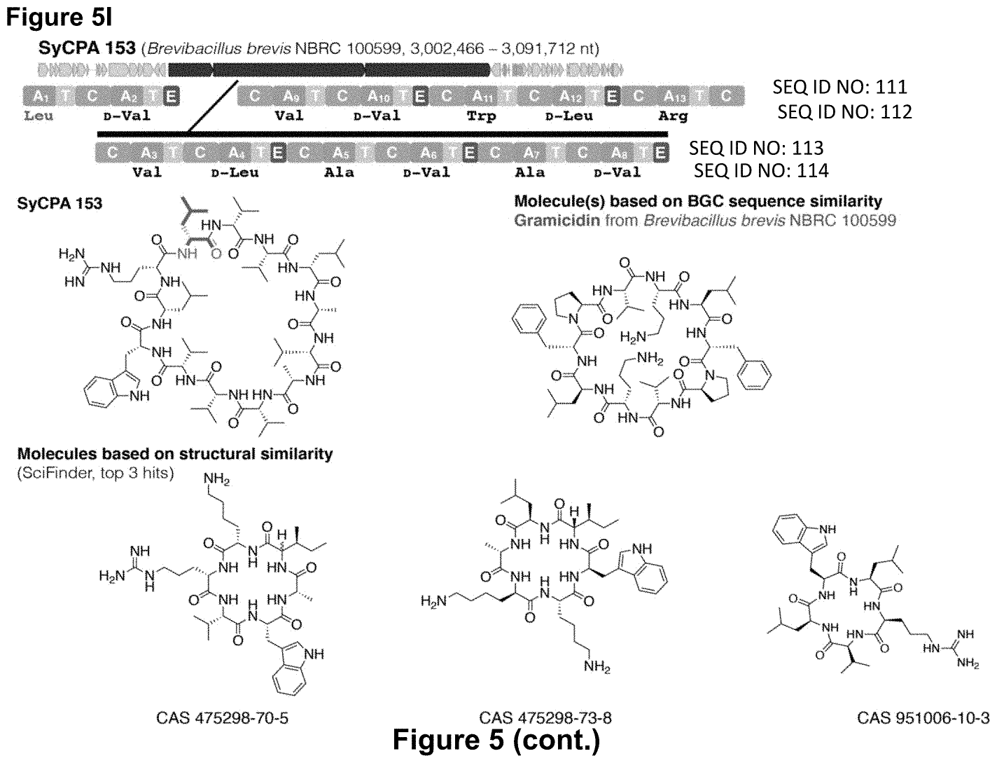

All nine SyCPAs were designed based on biosynthetic gene clusters that have not been previously associated with any natural products and their structures did not closely resemble any natural products deposited in the Dictionary of Natural Products or SciFinder databases (FIG. 5) (Dictionary of Natural Products, accessed March, 2020: dnp.chemnetbase.com/faces/chemical/ChemicalSearch.xhtml; Medema M H et al., 2015, Nat. Chem. Biol. 11, 625-631; SciFinder: A CAS solution, accessed January, 2020, scifinder.cas.org/). These SyCPAs ranged from 5 to 13 amino acids in length and contained 18 to 39-membered macrocycles (FIG. 3). Representatives of all three cyclization modes that were employed were found among the final hits. The largest peptide, SyCPA 153, was the lone head-to-tail macrocycle (cHT). Four peptides, each belonged to the other two types of cyclic modes, i.e., cyclization through the fatty acid (cFA, SyCPA 102, SyCPA 116, SyCPA 123, and SyCPA 144) and cyclization through a nucleophilic side-chain (cSC, SyCPA 2, SyCPA 4, SyCPA 12, and SyCPA 63). As opposed to being dominated by metabolites from Actinobacteria, which has been the most productive source of antibiotics from fermentation-based discovery efforts, the majority of SyCPAs were inspired by biosynthetic gene clusters found in the genomes of Proteobacteria and Firmicutes: four from Proteobacteria, three from Firmicutes, and two from Actinobacteria. As the syn-BNP approach focused on individual biosynthetic gene clusters and not entire organisms, it was not biased by the need for a bacterium to be “biosynthetically rich” to justify its inclusion into the screening platform, and therefore more likely to provide access to molecules inspired by “gene cluster poor” taxa that might otherwise be deprioritized in natural product screening programs.

### Mode of Action Studies

Although known cyclic peptide antibiotics have diverse modes of action, they have frequently been found to either inhibit cell wall biosynthesis by binding intermediates in this biosynthetic pathway or cause membrane disruption through depolarization or cell lysis (Lambert P A et al., 2002, Mol. Med. Microbiol., 1:591-598). Therefore each SyCPA was tested for these common modes of action using three discrete assays: SYTOX fluorescence (cell lysis), 3,3′-dipropylthiadicarboncyanine iodide (DiSC₃(5)) fluorescence (membrane depolarization) and UDP-MurNAc-pentapeptide accumulation (inhibition of cell wall biosynthesis).

Cell Lysis by the Broad Spectrum SyCPA 12, SyCPA 102, and SyCPA 123: Cell lysis was assessed using the nucleic acid binding dye SYTOX™. Antibiotics were tested for lytic activity against S. aureus unless they were significantly more active against B. subtilis, in which case B. subtilis was used (FIG. 4C and FIG. 4D). At 4× their MICs three broad spectrum antibiotics, SyCPA 12, SyCPA 102, and SyCPA 123, induced SYTOX fluorescence, indicating that they induced cell lysis. All three of these antibiotics contained positively charged residues. Cationic peptide antibiotics were a structurally diverse class of natural products that commonly function by interference with the cytoplasmic membrane barrier (Epand R M et al., 2016, Biochim. Biophys. Acta, 1858:980-987). They have attracted growing interest in recent years because they often exhibit activity against antibiotic resistant Gram-negative pathogens and show low rates of resistance (Lazar Vet al., 2018, Nat. Microbiol., 3:718-731). As SyCPA 12, SyCPA 102, and SyCPA 123 were structurally distinct from any previously reported natural products (FIG. 5), they provided novel chemical scaffolds for investigating the bioactivity of cationic peptide antibiotics.

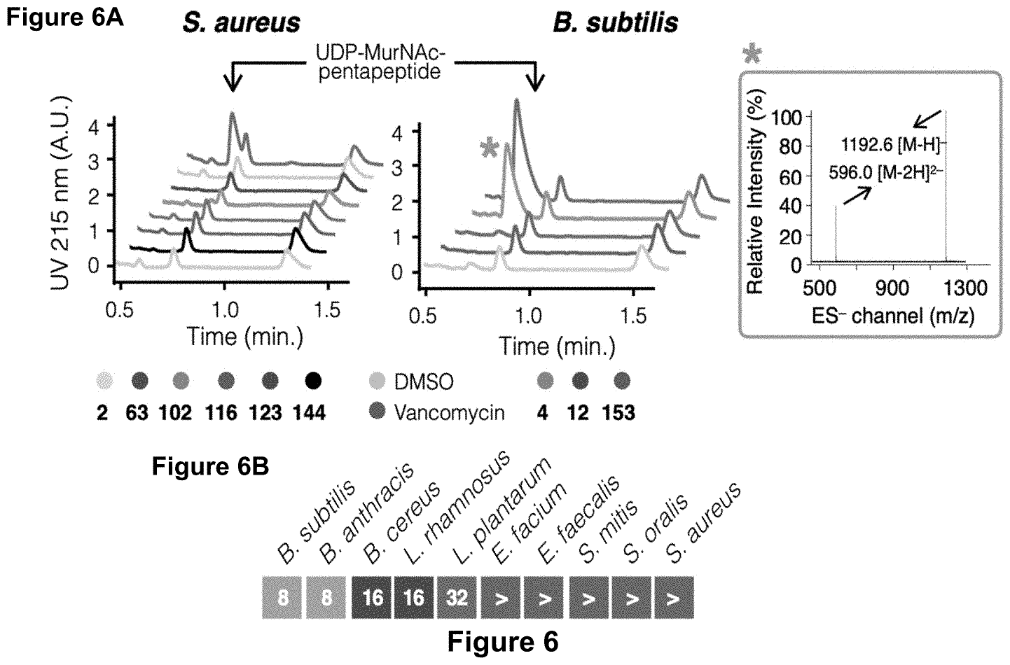

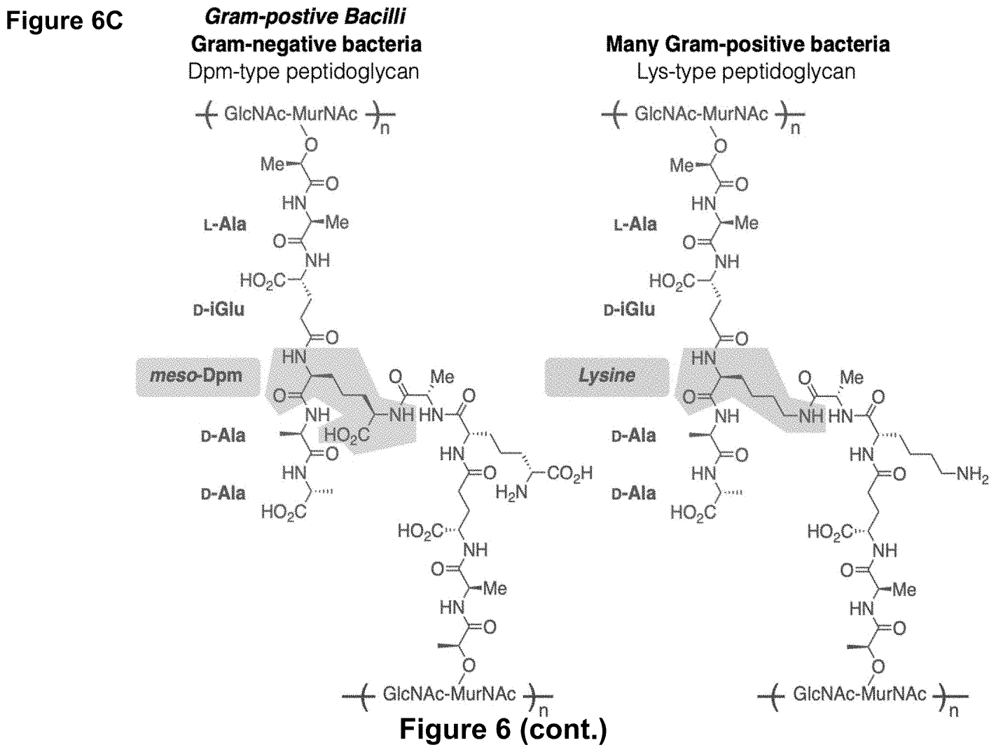

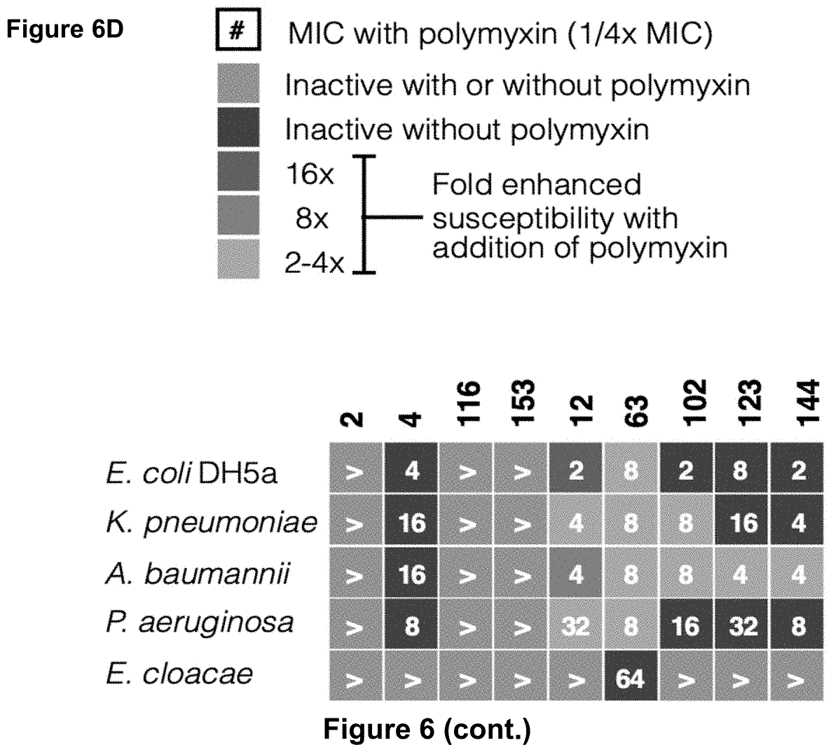

Inhibition of Cell Wall Biosynthesis by SyCPA 4: Antibiotics that bind cell wall biosynthetic intermediates often resulted in the accumulation of the lipid II precursor UDP-MurNAc-pentapeptide. Based on the spectrum activity of each SyCPA, cultures of either S. aureus or B. subtilis were exposed to individual SyCPAs and then monitored by LCMS for the accumulation of UDP-MurNAc-pentapeptide (FIG. 6A). B. subtilis cultures treated with SyCPA 4 containing increased concentrations of UDP-MurNAc-pentapeptide indicated inhibition of cell biosynthesis by this antibiotic (FIG. 6A). SyCPA 4 was most active against B. subtilis in the initial screen. When tested against a broader collection of bacteria it remained most potent against Gram-positive Bacilli (e.g., Bacillus anthracis, Bacillus cereus, and Lactobacillus rhamnosus, FIG. 6B) but largely inactive against other strains. The structure of the peptidoglycan in Gram-positive Bacilli differed from that in most other Gram-positive bacteria in that a meso-diaminopimelic acid replaced lysine at the third position of the pentapeptide moiety (FIG. 6C) (Firczuk M et al., 2007, FEMS Microbiol. Rev., 31:676-691), suggesting that SyCPA 4 antibiosis were likely dependent on an interaction with this divergent residue. Interestingly, Gram-negative peptidoglycan also contained meso-diaminopimelic acid; however, SyCPA 4 was not active against any of the Gram-negative bacteria that were initially tested. This was likely due to the Gram-negative outer membrane preventing access to the peptidoglycan. Therefore the effect of polymyxin, which disrupted the outer membrane of Gram-negative bacteria, were tested on the antibiosis of SyCPA 4, as well as the other SyCPAs. All SyCPAs with native activity against Gram-negative bacteria showed increased potency in the presence of polymyxin (FIG. 6D). Among the Gram-positive specific SyCPAs, SyCPA 4 was the only one to show an increased spectrum of activity. In fact, in combination with polymyxin, SyCPA 4 was active against most Gram-negative ESKAPE pathogens. While detailed binding assays are required to determine the exact mechanism of SyCPA 4's cell wall inhibition activity, its spectrum of activity suggested that it likely specifically interacted with the meso-diaminopimelic acid moiety that was common to the peptidoglycan of Gram-positive Bacilli as well as Gram-negative bacteria.

Interestingly, SyCPA 4 was also the only Gram-positive specific antibiotic that caused a dramatic increase in SYTOX fluorescence, suggesting that in addition to inhibiting cell wall biosynthesis it also induced cell lysis (FIG. 4C). This was reminiscent of the bifunctional semisynthetic glycolipopeptide telavancin that was recently approved for treatment of antibiotic resistant Gram-positive bacterial infections (Higgins D L et al., 2005, Antimicrob. Agents Chemother., 49:1127-1134). In the case of telavancin, the peptide moiety interacted with peptidoglycan to inhibit cell wall biosynthesis and the lipid substituent interacted with the bacterial membrane to cause depolarization. SyCPA 4 induced an increase in SYTOX fluorescence at 4×its MIC but had no such effect at 1× its MIC, suggesting that inhibition of cell wall biosynthesis was likely its principal mode of action except at very high concentrations. Bifunctional antibiotics were of considerable interest due to the low rates of resistance development they tend to exhibit (Pokrovskaya V et al., 2010, Expert Opin. Drug Discov., 5, 883-902). This mirrors the experience with SyCPA 4 in that all efforts to raise resistant mutants have so far been unsuccessful. SyCPA 4 was given the trivial name “gladiosyn” (Burkholderia gladioli BSR3 syn-BNP).

Membrane Depolarization and Mycobacterium Tuberculosis Growth Inhibition by SyCPA 63: Cell membrane depolarization was measured using the voltage-sensitive dye 3,3′-dipropylthiadicarboncyanine iodide (DiSC₃(5)). In addition to the four SyCPAs that caused cell lysis (SyCPA 4, SyCPA 12, SyCPA 102, and SyCPA 123), one additional antibiotic, SyCPA 63, induced an increase in DiSC₃(5) fluorescence (FIG. 6), indicating that it depolarized the bacterial membrane but did not lyse bacteria. SyCPA 63 showed the broadest spectrum of activity among the SyCPAs was identified. Among the Gram-negative bacteria that were tested, it was most active against K. pneumoniae (8 μg/mL), but also showed modest antibacterial activity against most of the Gram-negative bacteria in the panel. Interestingly, when tested for activity against other pathogens, SyCPA 63 was the only SyCPA that inhibited the growth of M. tuberculosis H37Rv (MIC 6 μg/mL) (FIG. 4A). SyCPA 63's broad spectrum of activity against diverse pathogens, together with its minimal HeLa cell cytotoxicity and the failure to identify SyCPA 63 resistant mutants in laboratory experiments, make it an appealing structure for future synthetic efforts designed to improve its potency. SyCPA 63 was given the trivial name thurinsyn (Bacillus thuringiensis BMB171 syn-BNP).

### Resistant Mutant Screening

To look for other potential mechanisms of action, it was attempted to raise resistant mutants for each antibiotic that did not strongly respond to any of the discrete assays used above (SyCPAs 2, SyCPAs 116, SyCPAs 144, and SyCPAs 153).

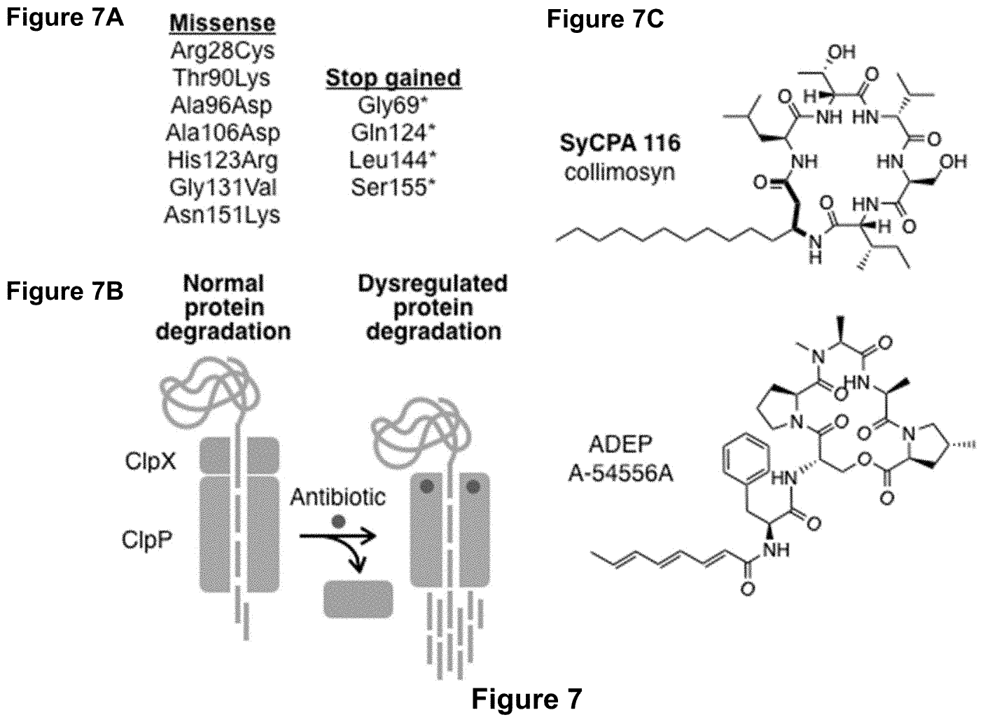

Dysregulation of the ClpP Protease by SyCPA 116: In an initial round of resistant mutant screening using S. aureus USA 300 all SyCPA 116 resistant mutants contained single-nucleotide polymorphisms (SNPs) in the farR gene (Table 2). FarR was a LysR transcription factor that induced expression of FarE, an efflux pump that has been associated with resistance to antimicrobial fatty acids (Alnaseri H et al., 2019, J. Bacteriol. 201). Constitutive overexpression of the farER effux system (pALCfarER) in an S. aureus farER deletion mutant (Alnaseri H et al., 2019, J. Bacteriol. 201) (SyCPA 116 MIC of 8 μg/mL) conferred resistance to SyCPA 116 (MIC>64 μg/mL), suggesting that FarE can efflux SyCPA 116. Therefore, in a second round of screening it was attempted to raise resistant mutants using the farER deletion strain. This yielded mutants that were able to grow on >128 μg/mL of SyCPA 116. Whole genome sequencing of these strains identified mutants with SNPs in the ClpP protease (clpP) gene or in an operon that encoded a multisubunit Na+/H+ antiporter that has been associated with tolerance to diverse molecules, including salt, cholate, and thrombin-induced platelet microbicidal protein (Bayer A S et al., 2006, J. Bacteriol., 188:211-222; Sannasiddappa T H et al., 2015, Infect. Immun., 83:2350-2357; Vaish M et al., 2018, J. Bacteriol. 200). Interestingly, a number of the clpP point mutations introduced stop codons (FIG. 7A), which suggested that disruption of this protease provided resistance to SyCPA 116. This was confirmed by the fact that a targeted clpP deletion mutant (S. aureus SH1000 ΔclpP) was resistant to SyCPA 116 (>128 μg/mL) but the parent of this strain was not (Frees D et al., 2003, Mol. Microbiol., 48:1565-1578).

TABLE 2
SNPs of Raised Resistant Mutants Compared to the Mother Strain.

| Mutant | POS | TYPE | REF | ALT | EFFECT | ANNOTATED GENE |
|---|---|---|---|---|---|---|
| 1 | 2692980 | SNP | T | C | Missense variant c. 346T>C | regulatory protein, |
| | | | | | p. Cys116Arg | TetR family |
| 2 | 2692980 | SNP | T | C | Missense variant c. 346T>C | regulatory protein, |
| | | | | | p. Cys116Arg | TetR family |
| 4 | 2692914 | SNP | G | T | Missense variant c. 280G>T | regulatory protein, |
| | | | | | p. Asp94Tyr | TetR family |
| 5 | 2692914 | SNP | G | T | Missense variant c. 280G>T | regulatory protein, |
| | | | | | p. Asp94Tyr | TetR family |
| 6 | 2692914 | SNP | G | T | Missense variant c. 280G>T | regulatory protein, |
| | | | | | p. Asp94Tyr | TetR family |
| 7 | 2692914 | SNP | G | T | Missense variant c. 280G>T | regulatory protein, |
| | | | | | p. Asp94Tyr | TetR family |
| 8 | 2692914 | SNP | G | T | Missense variant c. 280G>T | regulatory protein, |
| | | | | | p. Asp94Tyr | TetR family |
| 9 | 2692980 | SNP | T | C | Missense variant c. 346T>C | regulatory protein, |
| | | | | | p. Cys116Arg | TetR family |
| 10 | 2692914 | SNP | G | T | Missense variant c. 280G>T | regulatory protein, |
| | | | | | p. Asp94Tyr | TetR family |
| 11 | 2692977 | SNP | G | A | Missense variant c. 343G>A | regulatory protein, |
| | | | | | p. Val115Ile | TetR family |
| 12 | 2692980 | SNP | T | C | Missense variant c. 346T>C | regulatory protein, |
| | | | | | p. Cys116Arg | TetR family |
| 13 | 2692914 | SNP | G | T | Missense variant c. 280G>T | regulatory protein, |
| | | | | | p. Asp94Tyr | TetR family |
| 14 | 2692914 | SNP | G | T | Missense variant c. 280G>T | regulatory protein, |
| | | | | | p. Asp94Tyr | TetR family |
| 15 | 2692914 | SNP | G | T | Missense variant c. 280G>T | regulatory protein, |
| | | | | | p. Asp94Tyr | TetR family |
| 16 | 2692980 | SNP | T | C | Missense variant c. 346T>C | regulatory protein, |
| | | | | | p. Cys116Arg | TetR family |
| 17 | 2692980 | SNP | T | C | Missense variant c. 346T>C | regulatory protein, |
| | | | | | p. Cys116Arg | TetR family |
| 18 | 2692914 | SNP | G | T | Missense variant c. 280G>T | regulatory protein, |
| | | | | | p. Asp94Tyr | TetR family |
| 19 | 2692914 | SNP | G | T | Missense variant c. 280G>T | regulatory protein, |
| | | | | | p. Asp94Tyr | TetR family |
| 20 | 2692980 | SNP | T | C | Missense variant c. 346T>C | regulatory protein, |
| | | | | | p. Cys116Arg | TetR family |
| 21 | 2692914 | SNP | G | T | Missense variant c. 280G>T | regulatory protein, |
| | | | | | p. Asp94Tyr | TetR family |

The ClpP protease was part of a protein degradation machinery that targeted misfolded and unneeded proteins (Clarke A K, 1996, J. Biosci., 21:161-177). This system was not essential to S. aureus. Inhibition of ClpP was therefore not a viable antibacterial mode of action. On the other hand, dysregulation of ClpP that resulted in uncontrolled protein degradation was known to be lethal (FIG. 7B) (Kirstein J et al., 2009, EMBO Mol. Med., 1:37-49). The fact that deletion of clpP provided resistance suggested a ClpP dysregulation mode of action for SyCPA 116. Only one known family of bacterial natural product antibiotics, the acyl depsipeptides (ADEPs) function by dysregulating ClpP (Kirstein J et al., 2009, EMBO Mol. Med., 1:37-49; Hinzen B et al., 2006, Chem Med Chem, 1:689-693). Although both SyCPA 116 and the ADEPs were cyclic acylated peptides their structures are quite different (FIG. 7C): 1) they share no amino acids, 2) their macrocycles differ in size, 3) SyCPA 116 is not a depsipeptide but instead cyclized through an amide bond, and 4) the N-terminus of SyCPA 116 contains a fully saturated acyl substituent while ADEPs contain polyunsaturated acyl substituents. Whether ADEPs and SyCPA 116 bind the same site or even function by the exact same mechanism remains to be determined. Efforts to identify unique chemical matter that activated ClpP and killed bacteria have seen only modest success (Leung E et al., 2011, Chem. Biol., 18:1167-1178; Lavey N P et al., 2016, J. Nat. Prod., 79:1193-1197; Ye F et al., 2016, Mol. Biosyst., 13:23-31). As seen with some ADEP analogs, SyCPA 116 also showed human cell line toxicity (FIG. 4B). This was likely due to dysregulation of the mitochondrial ClpP protease, which was known to lead to apoptotic cell death (Wong K S et al., 2018, Cell Chem. Biol., 25:1017-1030 e1019). The SyCPA 116 scaffold therefore provided opportunities to explore ClpP protease activation not only as an antibacterial mode of action but also as a way of killing cancer cells. SyCPA 116 was given the name “collimosyn” (Collimonas fungivorans Ter331 syn-BNP).

SyCPA 2, SyCPA 144, and SyCPA 153: SyCPA 2, SyCPA 144, and SyCPA 153 were not highly active in any of the discrete assays that looked at initially, and resistant mutants that could provide insight into their modes of action were not able to be identified. Each of these antibiotics did cause a slight increase in either SYTOX or DiSC₃(5) fluorescence in the initial assays (FIG. 4C and FIG. 4D), suggesting limited cell lysis or membrane depolarization, respectively. Additional experiments are required to determine if this was indicative of the antibiotic's primary mode of action or whether this was a secondary effect. Although their modes of action remain to be determined, among these antibiotics, SyCPA 144 was of particular interest for future synthetic optimization studies due to its native activity against A. baumannii, broad spectrum activity in the presence of polymyxin, lack of toxicity to human cells in vitro and the inability to raise resistance mutants in laboratory-based experiments using either Gram-positive or Gram-negative bacteria (S. aureus, B. subtilis, and Escherichia coli imp). SyCPA 144 was named “mucilasyn” (Paenibacillus mucilaginosus syn-BNP).

Continuous improvement in DNA sequencing technologies promised to uncover an increasingly large number of uncharacterized bacterial biosynthetic gene clusters. The biosynthetic instructions contained in these gene clusters encode a reservoir of naturally occurring bioactivities, including the diverse mechanisms that bacteria have evolved to control the growth of other bacteria. The successful translation of uncharacterized biosynthetic gene clusters into bioactive molecules should have a significant positive impact on antibiotic discovery pipelines, and in turn on the ability to address the growing problem of antimicrobial resistance. Of the 96 gene clusters that were explored using the syn-BNP approach, almost ten percent inspired the synthesis of an antibiotic with activity against antibiotic-resistant pathogens of clinical interest, many of which failed to develop resistance in laboratory experiments. The data suggested that these new antibiotics function by multiple different modes of action.

Syn-BNPs should not represent perfect translations of the instructions contained in gene clusters but instead biosynthetically inspired structures with enough similarity to native metabolites to capture the diverse bioactivities encoded by uncharacterized gene clusters. In fact, because the vast majority of biosynthetic gene clusters were silent in laboratory conditions, it was not possible to determine the exact product of most biosynthetic gene clusters that inspired syn-BNPs. As has often been done with traditional natural products, mechanistically interesting syn-BNPs serve as inspiration for the generation of more potent and clinically relevant small molecule derivatives in subsequent synthetic optimization studies. The work outlined here adds to a growing body of evidence that the syn-BNP approach represents an effective, scalable and orthogonal method for using the biosynthetic instructions found in natural product biosynthetic gene clusters to inspire the creation of bioactive small molecules, in particular, antibiotics with diverse modes of action. Improvements in bioinformatic algorithms for predicting chemical structures from biosynthetic gene clusters together with the incorporation of additional synthetic complexity beyond peptide cyclization undoubtedly lead to even higher hit rates and more diverse bioactivities in future syn-BNP studies.

In summary, bacterial natural products have inspired the development of numerous antibiotics in use today. As resistance to existing antibiotics has become more prevalent, new antibiotic lead structures and activities were desperately needed. An increasing number of natural product biosynthetic gene clusters, to which no known molecules can be assigned, were found in genome and metagenome sequencing data. Here structural information encoded in this underexploited resource were accessed using a syn-BNP approach, which relied on bioinformatic algorithms followed by chemical synthesis to predict and then produce small molecules inspired by biosynthetic gene clusters. In total, 157 syn-BNP cyclic peptides inspired by 96 nonribosomal peptide synthetase gene clusters were synthesized and screened for antibacterial activity. This yielded nine antibiotics with activities against ESKAPE pathogens as well as Mycobacterium tuberculosis. Not only were antibiotic-resistant pathogens susceptible to many of these syn-BNP antibiotics, but they were also unable to develop resistance to these antibiotics in laboratory experiments. Characterized modes of action for these antibiotics included cell lysis, membrane depolarization, inhibition of cell wall biosynthesis, and ClpP protease dysregulation. Increasingly refined syn-BNP-based explorations of biosynthetic gene clusters should allow for the more rapid identification of evolutionarily inspired bioactive small molecules, in particular antibiotics with diverse mechanism of actions that could help confront the imminent crisis of antimicrobial resistance.

The materials and methods are now described.

### Chemical Reagents, Consumables, and Instruments

Pre-loaded 2-chlorotrityl resins for peptide syntheses were purchased from Matrix Innovation, Inc. (Quebec, Canada). Reagents for solid-phase peptide synthesis (SPPS), i.e., PyAOP ((7-azabenzotriazol-1-yloxy) tripyrrolidinophosphonium hexafluorophosphate), PyBOP ((benzotriazole-1-yloxy)tripyrrolidinophosphonium hexafluorophosphate), Cl-HOBt (6-chloro-1-hydroxy benzotriazole) were purchased from P3 BioSystems (Louisville, Ky.). Standard N-Fmoc amino acid building blocks were purchased from P3 BioSystems and Chem-Impex International (Wood Dale, Ill.), and building blocks with allyloxycarbonyl (Alloc) protected side-chains were purchased from Ark Pharm, Inc. (Arlington Heights, Ill.) and Chempep, Inc. (Wellington, Fla.). (D/L)-N-Fmoc-3-aminotetradecanoic acid was purchased from Chemieliva Pharmaceutical Co. (Chongqing, China). MTT (3-(4,5-dimethyl-thiazol-2-yl)-2,5-diphenyltetrazolium bromide), tetradecanoic acid, and Pd(PPh3)₄ (tetrakis(triphenylphosphine) palladium(0)), and solid-phase extraction (SPE)C-18 cartridges were purchased from Sigma-Aldrich (St. Louis, Mo.). A 12-position SPE vacuum manifold was manufactured by WXIntl (Rowland Heights, Calif.) and purchased on eBay. Fluorescent dyes SYTOX Green™ and DISC3(5) (3,3′-dipropylthiadicarbocyanine Iodide) were purchased from ThermoFisher Scientific (Waltham, Mass.) and the assay results were recorded using SpectraMax M2e (Molecular Devices, San Jose, Calif.). Peptide purification was performed on an)(Bridge Prep C-18 column (Waters Corporation, Milford, Mass.) using a 1200 HPLC system (Agilent Technologies, Santa Clara, Calif.). LCMS was performed on a Waters Acquity UPLC M-class system. All other reagents, solvents, and consumables were purchased from VWR International (Radnor, Pa.). The ClpP protease assay kit was purchased from ProFoldin (Hudson, Mass.) and a Tecan Infinite M Nano+ plate reader (Morrisville, N.C.) was used to measure protease degradation.

### Microbes, Cell Line, and Growth Media

Bacterial strains and growth conditions appear in Table 3. Growth media, including Luria-Bertani (LB), brain heart infusion (BHI) and tryptic soy broth (TSB), for microbes were purchased as premade powders from Becton Dickinson (Franklin Lakes, N.J.). Aerobic bacterial cultures were grown shaken (200 rpm) and cultures of facultative aerobic bacteria (Streptococcus and Lactobacillus species) were grown statically in an anaerobic chamber. Primary screening of Syn-BNP cyclic peptides was performed on bacteria embedded solid media (LB supplemented with 1.5% (w/v) agar) grown statically at 30° C.

TABLE 3
Strains and Growth Conditions.

| Species | Strain | Media | Temp. (° C.) |
|---|---|---|---|
| Acinetobacter baumannii | ATCC 17978 | LB | 37 |
| Bacillus anthracis | Sterne | TSB | 37 |
| Bacillus cereus | ATCC 14579 | LB | 30 |
| Bacillus subtilis | 168 1A1 | LB | 30 |
| Enterobacter cloacae | ATCC 13047 | LB | 37 |
| Enterococcus faecalis | OR1GF | LB | 37 |
| Enterococcus faecium | Com15 | LB | 37 |
| Escherichia coli | BAS849 | LB | 37 |
| Escherichia coli | DH5α | LB | 37 |
| Klebsiella pneumoniae | ATCC 10031 | LB | 37 |
| Lactobacillus rhamnosus | LMS2-1 | BHI | 30 |
| Lactobacillus Plantarum | WCFS1 | BHI | 30 |
| Mycobacterium tuberculosis | H37RV | Middlebrook 7H9 | 37 |
| Pseudomonas aeruginosa | PAO1 | LB | 37 |
| Staphylococcus aureus | USA300 | LB | 37 |
| Staphylococcus aureus | USA300 ΔfarER | LB | 37 |
| Staphylococcus aureus | USA300 ΔfarER pALCfarER | LB | 37 |
| Staphylococcus aureus | SH1000 | LB | 37 |
| Staphylococcus aureus | SH1000 ΔClpP | LB | 37 |
| Streptococcus mitis | F0392 | BHI | 30 |
| Streptococcus oralis | M7A | BHI | 30 |

### Syn-BNP Peptide Sequence Prediction

High quality complete bacterial genomes present in GenBank as of December 2014 (˜3,000) at the time this project was initiated were analyzed by using antiSMASH bacterial version 2.0 (Blin K et al., 2013, Nucleic Acids Res., 41:W204-212).

### SPPS and Peptide Cyclization

All target peptides were produced by standard Fmoc (fluorenylmethoxycarbonyl) based solid-phase synthesis on 2-chlorotrityl resins using standard amino acid building blocks. To facilitate side-chain cyclization reactions, serine and threonine nucleophiles were replaced with 2,3-diaminopropionic acid, an isosteric residue that contains a more reactive nitrogen nucleophile. Similarly, 3-aminomyristic acid was used as the fatty acid for generating N-acylated peptides. The side-chain amines on residues used as cyclization nucleophiles (e.g., diaminopropionic acid, ornithine or lysine) were protected with an allyloxycarbonyl group to permit palladium-catalyzed differential deprotection. The 3-amino-myristic acid in N-acylated peptides as well as the N-terminus of non-acylated peptides were protected with Fmoc to permit differential deprotection with piperidine. Syn-BNP cyclic peptides for primary screening were synthesized from their linear counterparts purchased as on-resin crude materials from Ontores Biotechnologies, Inc. (Hangzhou, China) and Tahepna Biotechnologies, Co. (Hangzhou, China). Peptides used in validation and follow up studies were synthesized in-house either through manual synthesis or using a Biotage Alstra Initiator-plus automated microwave synthesizer (Charlotte, N.C.). Syntheses were performed as described previously (Chu J et al., 2019, J. Am. Chem. Soc., 141:15737-15741) with the exception that tetradecanoic acid and N-Fmoc-3-aminotetradecanoic acid were used in the N-acylation of cSC and cFA peptide designs, respectively.

### Peptide Purification—Solid-Phase Extraction

C18 cartridges were mounted on a vacuum manifold that enables the purification of up to twelve peptides in parallel. Crude material from cyclization reactions were solubilized in the minimal amount of methanol (MeOH) and mixed with an equal volume of water. The resulting precipitate/suspension was immediately loaded onto a C18 cartridge that had been pre-washed with MeOH (10 mL) and water (10 mL). The cartridge was washed with 50% MeOH (10 mL), acidified 50% MeOH (1% v/v formic acid), eluted with 100% MeOH, and the eluted MeOH fraction was dried by using a speedvac. Global deprotection was accomplished by treating the cyclic peptides with trifluoroacetic acid (TFA) containing water and triisopropylsilane (2.5% (v/v) of each) at room temperature for 2 h. Liquid chromatography coupled with low-resolution mass spectrometry (LCMS) was used to verify the presence of the desired Syn-BNP cyclic peptide once TFA had evaporated. This crude material was dissolved in DMSO (12.8 mg/mL) and used directly in Primary Antimicrobial Screening.

### Cyclic Peptide Purification— HPLC

Crude Syn-BNP cyclic peptides that showed antimicrobial activity were purified on a XBridge Prep C18 HPLC column (130 Å, 5 μm, 10×250 mm) using a dual solvent system (A/B: water/acetonitrile, supplemented with 0.1% v/v of formic acid). Most peptides were eluted 35 to 75% B. Pure peptides were dissolved in DMSO at 12.8 mg/L for MIC measurements and mechanism of action studies. Diastereomers of antibiotic hits (if separable) were tested as separate entities and the more active isomer was used in MOA studies.

### Primary Antimicrobial Screening

Single colonies of bacteria were inoculated in LB broth and grown overnight at 200 rpm in a shaking incubator at 37° C. The overnight culture was used to re-inoculated fresh LB broth ( 1/200×), grown to late log phase (OD₅₉₅˜1.0), and mixed with molten LB/agar (1.5% (w/v), 50 mL) at 55° C. The mixture was poured onto a petri dish (150×15 mm) and allowed to cool down to room temperature inside a laminar flow hood. DMSO solutions of 4 μL of each Syn-BNP cyclic peptides at 12.8 and 4.27 μg/μL were spotted directly on solidified LB/agar embedded with bacteria. Alongside Syn-BNPs, three known antibiotics (carbenicillin, chloramphenicol, kanamycin) and DMSO were used as the positive and negative controls, respectively. Once the DMSO droplets have been absorbed into the solid media, the petri dishes were incubated statically at 30° C. for 18 h. On the following day, the petri dishes were visually inspected, and each Syn-BNP was given a semi-quantitative score on the scale of 0 (inactive) to 3 (potent) based on the size and clarity of its corresponding growth inhibition zone.

### MIC Determination

Standard susceptibility assays were performed in the appropriate growth medium in 96-well microtiter plates to determine the MIC by the broth microdilution method in accordance to protocols recommended by Clinical and Laboratory Standards Institute (Weinstein MP, 2012, Methods for dilution antimicrobial susceptibility tests for bacteria that grow aerobically, 9th edition; Clinical and Laboratory Standards Institute, Wayne, Pa.). For polymyxin-supplemented assays, the MIC of polymyxin against a bacterium was first determined (lx MIC). The growth medium for setting up a MIC assay was then supplemented with polymyxin at ¼×MIC of each respective bacteria. The measured MICs were as follows: E. coli DH5α (0.0625 μg/mL), K. pneumoniae and P. aeruginosa (0.15 μg/mL), A. baumannii (0.3 μ/mL), and E. cloacae (2 μg/mL). Note that the E. cloacae ATCC 13047 strain, which was used was not susceptible to polymyxin (MIC>64 μg/mL). These supplemented growth media was then used in a standard broth microdilution to determine the polymyxin-supplemented MIC of each antibiotic hit. All assays were done at least in duplicate (n=2).

### Membrane Depolarization Assay

Membrane depolarization assays were done in a 384-well plate and all stock solutions were prepared in Dulbecco's phosphate buffer saline (PBS). An overnight bacterial culture was harvested by centrifugation, washed twice with PBS, and resuspended in PBS (OD₅₉₅˜0.4). Cell suspension (10 μL) and 20 μM DiSC₃(5) (5 μL) were added to PBS (15 μL) and incubated in the dark at room temperature for 15 min. Potassium chloride (2 M, 5 μL) was added and incubated for another 15 min. This mixture was then transferred into a 384-well microtiter plate and its fluorescence intensity was recorded continually at 3 sec intervals (ex/em 643/675 nm). Once the signal had stabilized, which takes approximately 3 to 5 min., Syn-BNP antibiotics were added and immediately mixed by manual pipetting without stopping recording. The final mixture is a PBS solution containing DiSC₃(5) (1 μM), KCl (100 mM), bacteria (OD₅₉₅˜0.04), and antibiotics at 1×MIC against the bacteria of interest. Gramicidin and DMSO were used as the positive and negative controls, respectively. All assays were done in duplicate (n=2); a representative recording for each Syn-BNP is shown in either FIG. 4C or FIG. 4D.

### Membrane Lysis Assay

Membrane lysis assays were done in disposable plastic cuvettes and all stock solutions were prepared in LB broth. An overnight bacterial culture was harvested by centrifugation and resuspended in fresh LB (OD₅₉₅˜0.4). SYTOX Green™ (15 μM, 100 μL) was added to the cell suspension (900 μL) and incubated in the dark at room temperature for 10 min. Fluorescence intensity of the mixture was recorded continually at 2 sec intervals (ex/em 488/523 nm). Once the signal had stabilized, which takes approximately 3 to 5 min., syn-BNP antibiotics as a 12.8 mg/mL DMSO solution was added and immediately mixed by manual pipetting without stopping the recording. The final mixture is a LB solution containing SYTOX (1.5 μM), bacteria (OD₅₉₅˜0.04), and antibiotics at 1×MIC against the bacteria of interest. Daptomycin supplemented with 10 mM calcium chloride (CaCl₂) and DMSO were used as the negative control and positive controls. Data were presented as the relative intensity with respect to the average fluorescence signal prior to the addition of the antibiotic. All assays were done in duplicate (n=2); a representative recording for each Syn-BNP is shown in either FIG. 4C or FIG. 4D.

### Lipid II Precursor Accumulation Assay

A single bacterial colony was inoculated into LB and grown overnight at 200 rpm in a shaking incubator at 37° C. On the following day, the overnight culture was used to re-inoculate fresh LB broth ( 1/200×) and grown to mid log phase (OD₅₉₅˜0.5). Chloramphenicol was added to 1 mL of mid log phase culture and incubated at 37° C. for 20 min at 200 rpm. Antibiotics of interest were added at 20 μg/mL and incubated for another 60 min. Cells were collected by centrifugation, resuspended in 30 μL of water, and then incubated in boiling water for 15 min. The boiled suspension was centrifuged at 15,000 g and the supernatant was analyzed by LCMS. Vancomycin and DMSO were used as the positive and negative controls, respectively. All assays were done in duplicate (n=2); a representative trace for each Syn-BNP was shown in FIG. 6A.

### Cytotoxicity Assessment

HeLa cells were grown in Dulbecco's Modified Eagle Medium (DMEM) supplemented with penicillin (10 units/mL), streptomycin (10 units/mL), L-glutamate (2 mM), and heat-inactivated fetal bovine serum (10% v/v) at 37° C. in a 5% CO₂ atmosphere. HeLa cells were seeded into 384-well plates (500 cells per well) and incubated in DMEM at 37° C. with 5% CO₂ for 16 h to allow cells to adhere. The OD₅₇₀ readings were used to calculate relative growth (%) based on the positive (2 μM Taxol™, 0%) and negative (DMSO, 100%) controls. The cytotoxicity assay was performed in quadruplicate (n=4) and analyzed according to protocol reported previously (Chu Jet al., 2019, J. Am. Chem. Soc., 141:15737-15741).

Resistant Mutant Selection and SNP Identification (Chu J et al., 2016, Nat. Chem. Biol., 12:1004-1006)

Resistant mutants were raised as reported previously. Briefly, a single bacterial colony was inoculated into LB and grown overnight at 200 rpm in a shaking incubator at 37° C. A portion of the overnight culture containing approximately 10\hat{ }9 cells was diluted ( 1/100× to 1/400× fold) into LB containing the antibiotic of interest at 2.5× of its MIC. The resulting mixture was distributed into microtiter plates at 200 μL per well. After incubating statically at 30° C. for 12 to 18 h, colonies that appeared were transferred into fresh LB containing the same concentration of antibiotic. In a second round of selection these cultures were grown at 30° C. for an additional 12 to 18 h. Mature cultures were struck for singles on LB/agar free of antibiotics. The MIC of twelve to thirty-six individual colonies was then tested using the standard microtiter dilution method described above. DNA was extracted from cultures of colonies that showed elevated MIC, and the resulting DNA was sequenced using MiSeq Reagent Kit V3 (MS-102-3003, Illumina). DNA extraction and next generation sequencing were performed as reported previously. SNPs were identified using SNIPPY (github.com/tseemann/snippy) by mapping MiSeq reads to the reference genome of S. aureus USA300_FPR3757 (RefSeq assembly accession: GCF_000013465.1).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound having the structure selected from the group consisting of 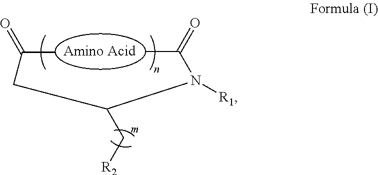
or a pharmaceutically acceptable salt, solvate, or tautomer thereof, 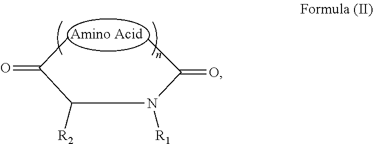
or a pharmaceutically acceptable salt, solvate, or tautomer thereof, and 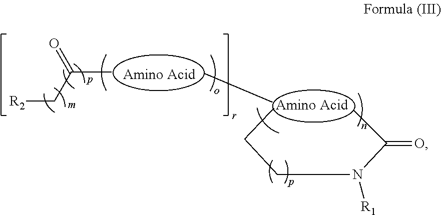
or a pharmaceutically acceptable salt, solvate, or tautomer thereof;
wherein each occurrence of R₁ and R₂ is independently selected from the group consisting of hydrogen, hydroxyl, hydroxylalkyl, amino, aminoalkyl, carboxyl, alkyl, cycloalkyl, heterocycloalkyl, aryl, arylalkyl, heteroaryl, heteroarylalkyl, carbamate, guanidine, and guanidine alkyl;
each occurrence of m and o is independently an integer from 0 to 100;
each occurrence of n and r is independently an integer from 1 to 100; and
each occurrence of p is independently an integer from 0 to 3.

2. The compound of claim 1, wherein the compound having the structure of Formula (III) is a compound having the structure selected from the group consisting of 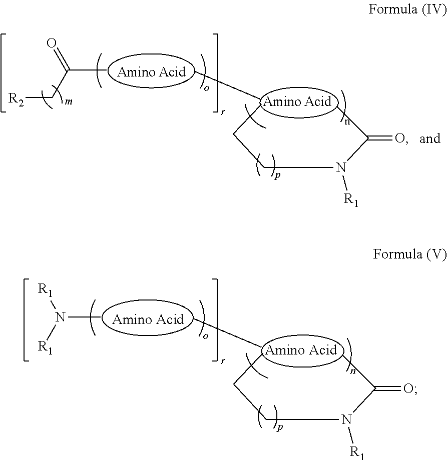
wherein each occurrence of R₁ and R₂ is independently selected from the group consisting of hydrogen, hydroxyl, hydroxylalkyl, amino, aminoalkyl, carboxyl, alkyl, cycloalkyl, heterocycloalkyl, aryl, arylalkyl, heteroaryl, heteroarylalkyl, carbamate, guanidine, and guanidine alkyl;
each occurrence of m and o is independently an integer from 0 to 20;
each occurrence of n and r is independently an integer from 1 to 20; and
each occurrence of p is independently an integer from 0 to 3.

3. The compound of claim 1, wherein the compound having the structure of Formula (I) is a compound having the structure selected from the group consisting of 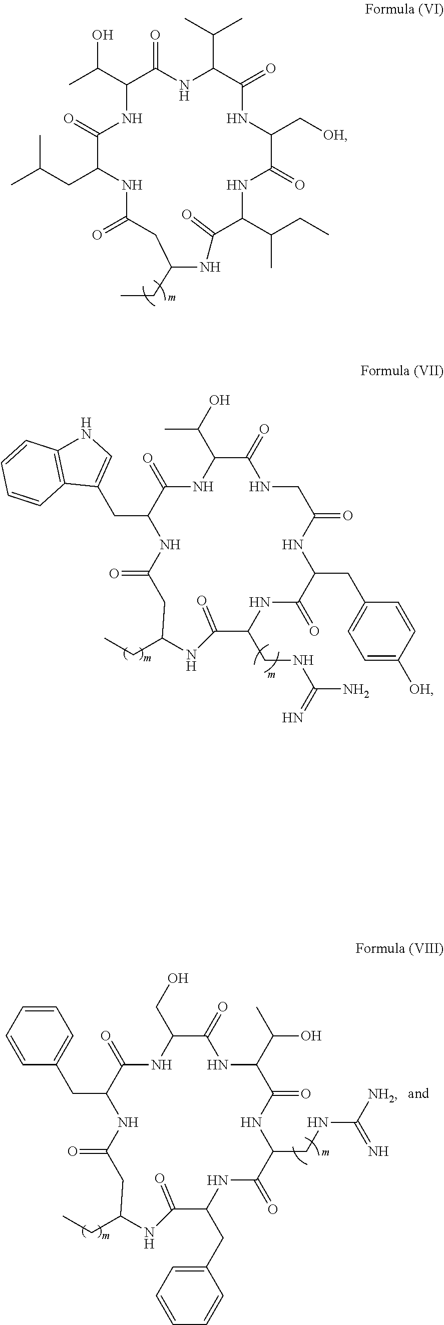 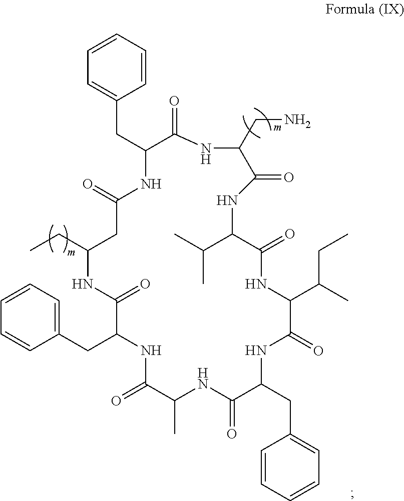
wherein each occurrence of m is independently an integer from 1 to 20.

4. The compound of claim 1, wherein the compound having the structure of Formula (II) is a compound having the structure of Formula (X) 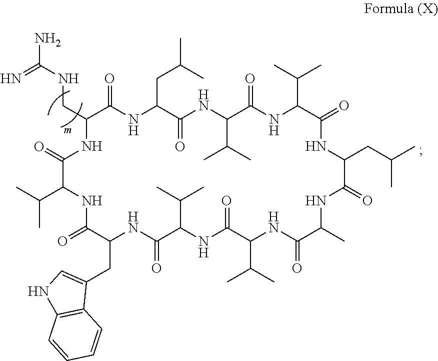
wherein each occurrence of m is independently an integer from 1 to 20.

5. The compound of claim 1, wherein the compound having the structure of Formula (III) is a compound having the structure selected from the group consisting of 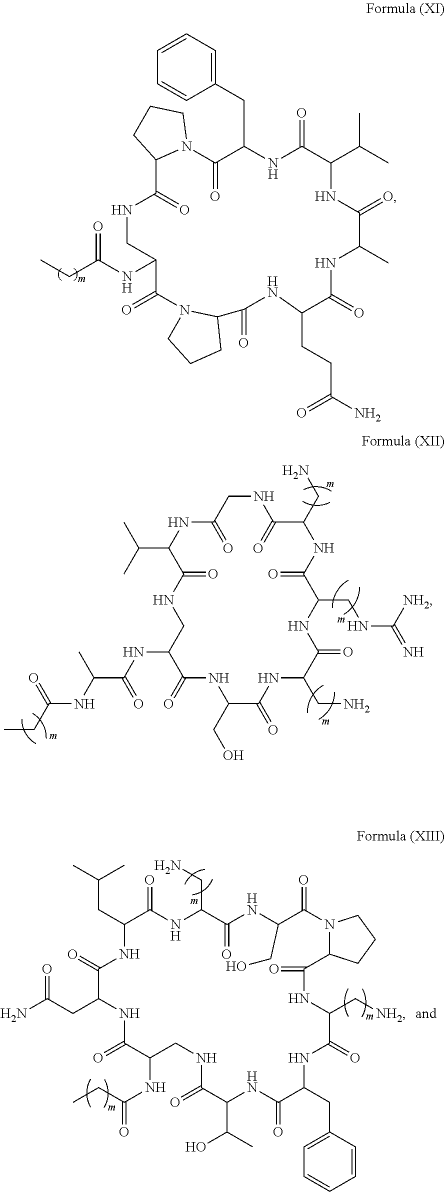 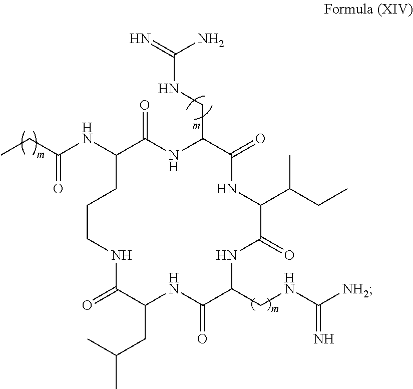
wherein each occurrence of m is independently an integer from 1 to 20.

6. The compound of claim 1, wherein
each occurrence of R₁ and R₂ is independently selected from the group consisting of hydrogen, hydroxylalkyl, aminoalkyl, alkyl, arylalkyl, heteroarylalkyl, and guanidine alkyl;
each occurrence of m and o is independently an integer from 0 to 15;
each occurrence of n and r is independently an integer from 1 to 15; and
each occurrence of p is independently an integer from 0 to 2.

7. The compound of claim 1, wherein the compound inhibits the growth of at least one microorganism.

8. The compound of claim 7, wherein the compound inhibits the growth of at least one microorganism at a minimal inhibitory concentration (MIC) between around 1 μg/mL and 10,000 μg/mL.

9. The compound of claim 8, wherein the compound inhibits at least one microorganism at a MIC around 8 μg/mL.

10. The compound of claim 7, wherein the microorganism is resistant to at least one antibiotic.

11. The compound of claim 1, wherein the compound is an antimicrobial compound.

12. The compound of claim 1, wherein the compound is a nonribosomal peptide.

13. The compound of claim 1, wherein the compound is a synthetic-bioinformatic natural product (syn-BNP).

14. The compound of claim 13, wherein the compound is a syn-BNP cyclic peptide antibiotics (syCPA).

15. A composition comprising at least one compound of claim 1.

16. The composition of claim 15, wherein the compound
a) reduces the growth of at least one microorganism;
b) reduces cell wall biosynthesis;
c) induces cell lysis;
d) induces membrane depolarization;
e) dysregulates at least one mitochondrial ClpP protease; or
any combination thereof.

17. A method of preventing or reducing the growth or proliferation of a microorganism, wherein the method comprises contacting the microorganism with a compound of claim 1 or a composition thereof.

18. The method of claim 17, wherein the microorganism is selected from the group consisting of a bacterium, virus, fungus, parasite, and any combination thereof.

19. The method of claim 17, wherein the microorganism is resistant to at least one antibiotic.

20. A method of treating or preventing a disease or disorder in a subject in need thereof, wherein the method comprises administering a therapeutically effective amount of at least one compound of claim 1 or a composition thereof to the subject.

21. The method of claim 20, wherein the disease or disorder is a disease or disorder associated with at least one microorganism.

22. The method of claim 21, wherein the disease or disorder associated with at least one microorganism is a bacterial infection.

23. The method of claim 22, wherein the bacterial infection is caused by a bacterium selected from the group consisting of Acinetobacter baumannii, Actinomyces israelii, Bacillus anthracis, Bacillus cereus, Bacillus subtilis, Bacteroides fragilis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtherias, Ehrlichia canis, Ehrlichia chaffeensis, Enterobacter cloacae, Enterococcus faecalis, Enterococcus faecium, Escherichia coli, Francisella tularensis, Haemophilus influenzae, Helicobacter pylori, Klebsiella pneumoniae, Lactobacillus rhamnosus, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Proteus mirabills, Pseudomonas aeruginosa, Nocardia asteroids, Rickettsia rickettsia, Salmonella enterica, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Candida albicans, Candida tropicalis, Candida glabrata, Candida parapsilosis, Candida krusei, Candida lusitaniae, Candida kefyr, Candida guilliermondii, and Candida dubliniensis, Yersinia pestis, Yersinia enterocolitica, Yersinia pseudotuberculosis, and any combination thereof.

24. The method of claim 23, wherein the bacterial infection is caused by a bacterium selected from the group consisting of Acinetobacter baumannii, Bacillus anthracis, Bacillus cereus, Bacillus subtilis, Enterobacter cloacae, Escherichia coli, Enterococcus faecium, Klebsiella pneumoniae, Lactobacillus rhamnosus, Listeria monocytogenes, Mycobacterium tuberculosis, Proteus mirabills, Pseudomonas aeruginosa, Salmonella enterica, Staphylococcus aureus, Staphylococcus epidermidis, and any combination thereof.

25. The method of claim 20, wherein the compound or composition thereof
a) reduces the growth of at least one microorganism;
b) reduces cell wall biosynthesis;
c) induces cell lysis;
d) induces membrane depolarization;
e) dysregulates at least one mitochondrial ClpP protease; or
any combination thereof.

26. The method of claim 25, wherein the microorganism is selected from the group consisting of a bacterium, virus, fungus, parasite, and any combination thereof.

27. The method of claim 26, wherein the bacterium is selected from the group consisting of Acinetobacter baumannii, Bacillus anthracis, Bacillus cereus, Bacillus subtilis, Enterobacter cloacae, Escherichia coli, Enterococcus faecium, Klebsiella pneumoniae, Lactobacillus rhamnosus, Listeria monocytogenes, Mycobacterium tuberculosis, Proteus mirabills, Pseudomonas aeruginosa, Salmonella enterica, Staphylococcus aureus, Staphylococcus epidermidis, and any combination thereof.

28. The method of claim 20, wherein the disease or disorder is a cancer.

29. A method of preparing a syn-BNP, wherein the method comprises:
a) analyzing nonribosomal peptide synthase (NRPS) gene clusters, wherein the NRPS gene clusters encode one or more peptides;
b) identifying one or more peptides that are encoded by the NRPS gene clusters, wherein the peptides comprise at least 4 amino acids;
c) synthesizing the peptides; and
d) covalently cyclizing the peptides to generate at least one syn-BNP.

30. A method of preparing a syCPA, wherein the method comprises:
a) analyzing NRPS gene clusters, wherein the NRPS gene clusters encode one or more peptides;
b) identifying one or more peptides that are encoded by the NRPS gene clusters, wherein the peptides comprise at least 4 amino acids;
c) synthesizing the peptides;
d) covalently cyclizing the peptides to generate at least one syn-BNP;
e) exposing the syn-BNP to at least one bacterium; and
f) identifying the syn-BNP that reduces the level of at least one bacterium.
